(19) United States
(12) Patent Application Publication
(10) Pub. No.: US 2025/0325585 A1
(43) Pub. Date: Oct. 23, 2025

(54) COMPOSITIONS AND METHODS FOR REDUCING CELL THERAPY IMMUNOGENICITY

(71) Applicant: The General Hospital Corporation, Boston, MA (US)

(72) Inventors: Marcela V. Maus, Lexington, MA (US); Korneel Grauwet, Boston, MA (US)

(73) Assignee: The General Hospital Corporation, Boston, MA (US)

(21) Appl. No.: 18/856,592

(22) PCT Filed: Apr. 14, 2023
(86) PCT No.: PCT/US2023/065784

§ 371 Date: Oct. 11, 2024

Related U.S. Application Data
(60) Provisional application No. 63/331,773, filed on Apr. 15, 2022.

(51) Int. Cl. A61K 35/17; A61K 31/11; A61K 40/31; A61K 40/42; A61P 35/02; A61P 37/06; C07K 14/005; C07K 16/28; C12N 15/113

(52) U.S. Cl. CPC A61K 35/17; A61K 31/11; A61K 40/31; A61K 40/4211; A61P 35/02; A61P 37/06; C07K 14/005; C07K 16/2803; C12N 15/113; A61K 2239/13; A61K 2239/21; A61K 2239/22; A61K 2239/48; C12N 2310/14; C12N 2310/531; C12N 2710/16022

(57) ABSTRACT

This application provides, in part, methods and compositions for decreasing the immunogenicity of cell therapies (e.g., CAR-T cell therapies) using inhibitors of transporter associated with antigen processing (TAPi) and oligonucleotides that decrease the expression of an immunogenic proteins (e.g., MHC Class I and Class II).

### RELATED APPLICATION

This application claims the benefit under 35 U.S.C. § 119(e) to U.S. Provisional Application No. 63/331,773, filed Apr. 15, 2022, the entire contents of which are incorporated herein by reference.

### GOVERNMENT SUPPORT

This invention was made with government support under Grant No. 5R01CA238268-03, awarded by The National Institutes of Health. The government has certain rights in the invention.

### BACKGROUND

Adoptive cell therapy, such as chimeric antigen receptor (CAR) CAR-T cell therapy, has revolutionized cancer treatment. However, clinical studies demonstrate that some patients develop humoral and cellular anti-CAR immune responses to non-self components of the CAR, limiting CAR-T cell persistence and the success of administering multiple doses. The potential for CAR-T cell rejection is even greater when using allogeneic immune effector cell products.

### SUMMARY

This application discloses methods and compositions for decreasing a subject's immune response to adoptive cell therapies. In some aspects, the disclosure is directed to the discovery that a subject's immune response to adoptive cell therapies (e.g., CAR-T cells) can be reduced by engineering the cells of the adoptive cell therapy to express an inhibitor of transporter associated with antigen processing (TAPi) which decreases expression of MHC class I. The disclosure is further directed to the discovery that a subject's immune response can additionally or alternatively be reduced by decreasing the expression of MHC class II (e.g. using RNAi targeting a MHC class II transactivator protein). Methods and compositions of the disclosure based on these discoveries do not require deep host immune suppression or complex gene editing and therefore avoid the disadvantages associated with previous methods that rely on such host immune suppression and/or gene editing. Further, in some embodiments, CAR-T cells expressing a TAPi and RNAi targeting MHC class II do not have increased susceptibility (relative to previous methods) of the therapeutic immune effector cells (IEC) to NK cell-mediated rejection, which is risk associated with current methods of β2M knockout.

In some aspects, this application discloses a cell comprising: (i) an inhibitor of transporter associated with antigen processing (TAPi) or variant thereof; and(ii) an oligonucleotide that is complementary to a polynucleotide encoding a MHC class II transactivator protein or variant thereof, wherein the oligonucleotide is selected from the group consisting of a RNA interference (RNAi) oligonucleotide, an antisense oligonucleotide (ASO), or a CRISPR interference (CRISPRi) oligonucleotide.

In some aspects, this application discloses a cell comprising: (i) an chimeric antigen receptor (CAR); and (ii) an inhibitor of transporter associated with antigen processing (TAPi) or variant thereof, and/or (iii) an oligonucleotide that is complementary to a polynucleotide encoding a MHC class II transactivator protein or variant thereof, wherein the oligonucleotide is selected from the group consisting of a RNA interference (RNAi) oligonucleotide, an antisense oligonucleotide (ASO), or a CRISPR interference (CRISPRi) oligonucleotide. In some embodiments, the oligonucleotide is complementary to any one of SEQ ID NOs: 7-12. In some embodiments, the oligonucleotide is complementary to SEQ ID NO: 7.

In some embodiments, the TAPi or variant thereof decreases expression of MHC class I.

In some embodiments, the TAPi is a viral TAPi. In some embodiments, the TAPi is a Herpesvirus TAPi. In some embodiments, the TAPi is selected from the group consisting of a Herpes Simplex virus (HSV) TAPi, Human Cytomegalovirus (HCMV) TAPi, or Epstein-Barr virus (EBV) TAPi. In some embodiments, the TAPi is selected from the group consisting of a Herpes Simplex virus (HSV) ICP47 TAPi, Human Cytomegalovirus (HCMV) US6 TAPi, or Epstein-Barr virus (EBV) BNLF2a TAPi. In some embodiments, the TAPi comprises an amino acid sequence that is at least 85% identical to any one of SEQ ID NOs: 1-3. In some embodiments, the TAPi comprises an amino acid sequence of any one of SEQ ID NOs: 1-3. In some embodiments, the RNAi oligonucleotide is selected from the group consisting of a siRNA, a miRNA or a shRNA. In some embodiments, the RNAi oligonucleotide is a shRNA. In some embodiments, the shRNA comprises a nucleic acid sequence of SEQ ID NO: 3. In some embodiments, the shRNA comprises a nucleic acid sequence of SEQ ID NO: 13. In some embodiments, the cell is a eukaryotic cell. In some embodiments, the cell is an immune cell. In some embodiments, the immune cell is a T cell.

In some embodiments, the cell further comprises a chimeric antigen receptor (CAR). In some embodiments, wherein the CAR comprises: (i) an extracellular target binding domain; (ii) a transmembrane domain; and (iii) an intracellular signaling domain. In some embodiments, the extracellular target binding domain binds to any one of CD19, CD79b, TACI, BCMA, MUC1, MUC16, B7H3, mesothelin, CD70, PSMA, PSCA, EGFRvIII, claudin6, binds to any pair of CD19/CD79b, BCMA/TACI, or is a TriPRIL antigen binding domain. In some embodiments, the extracellular target binding domain binds to CD19. In some embodiments, the extracellular target binding domain is not derived from a human polypeptide sequence. In some embodiments, the extracellular target binding domain is derived from a murine polypeptide sequence. In some embodiments, extracellular target binding domain comprises a VH amino acid sequence that has at least 85% identify to SEQ ID NO: 39 and a VL amino acid sequence that has at least 85% identify to SEQ ID NO: 40. In some embodiments, the transmembrane domain is selected from the group consisting of alpha chain of a T cell receptor, beta chain of a T cell receptor or zeta chain of a T cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), 4-1BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD1 Id, ITGAE, CD103, ITGAL, CD11a, LFA-1, ITGAM, CD11b, ITGAX, CD11c,ITGB1, CD29, ITGB2, CD18, LFA-1, ITGB7, TNFR2, DNAM1 (CD226), SLAMF4 (CD244, 2B4), CD84, CD96 (Tactile), CEACAMI, CRT AM, Ly9 (CD229), CD160 (BY55), PSGL1, CD100 (SEMA4D), SLAMF6 (NTB-A, Lyl08), SLAM (SLAMFI, CD150, IPO-3), BLAME (SLAMF8), SELPLG (CD162), LTBR, PAG/Cbp, NKp44, NKp30, NKp46, NKG2D, and/or NKG2C. In some embodiments, the intracellular signaling domain is selected from the group consisting of CD28, 4-1BB, CD27, TCR-zeta, FcR-gamma, FcR-beta, CD3-gamma, CD3-theta, CD3-sigma, CD3-eta, CD3-epsilon, CD3-zeta, CD22, CD79a, CD79b, and CD66d.

In some embodiments, the CAR comprises an amino acid sequence having at least 85% identify to SEQ ID NO: 41 and a nucleic acid sequence having at least 85% identity to SEQ ID NO: 17 or 18. In some aspects, this application discloses, a polynucleotide comprising a nucleic acid sequence encoding (i) a TAPi or variant thereof and (ii) an oligonucleotide that is complementary to a gene encoding a MHC class II transactivator protein. In some embodiments, the TAPi is a viral TAPi. In some embodiments, the TAPi or variant thereof decreases expression of MHC class I. In some embodiments, the TAPi is a Herpes Simplex Virus (HSV) TAPi. In some embodiments, the TAPi is selected from the group consisting of a Herpes Simplex virus (HSV) TAPi, Human Cytomegalovirus (HCMV) TAPi, or Epstein-Barr virus (EBV) TAPi. In some embodiments, the TAPi is selected from the group consisting of a Herpes Simplex virus (HSV) ICP47 TAPi, Human Cytomegalovirus (HCMV) US6 TAPi, or Epstein-Barr virus (EBV) BNLF2a TAPi. In some embodiments, the TAPi comprises an amino acid sequence that is at least 85% identical to any one of SEQ ID NOs: 1-3. In some embodiments, the TAPi comprises an amino acid sequence of any one of SEQ ID NOs: 1-3. In some embodiments, the oligonucleotide is complementary to any one of SEQ ID NOs: 7-12 or a variant thereof. In some embodiments, the oligonucleotide is complementary to SEQ ID NO: 7 or a variant thereof.

In some embodiments, the oligonucleotide is selected from the group consisting of a RNAi oligonucleotide or a CRISPR interference guide RNA. In some embodiments, the RNAi oligonucleotide is selected from the group consisting of a siRNA, a miRNA or a shRNA. In some embodiments, the RNAi oligonucleotide is an shRNA. In some embodiments, the shRNA is encoded by a nucleic acid sequence comprising of SEQ ID NO: 13.

In some embodiments, The polynucleotide further comprises a nucleic acid sequence encoding chimeric antigen receptor (CAR). In some embodiments, the CAR comprises: (i) an extracellular target binding domain; (ii) a transmembrane domain; and (iii) an intracellular signaling domain. In some embodiments, the extracellular target binding domain binds to any one of CD19, CD79b, TACI, BCMA, MUC1, MUC16, B7H3, mesothelin, CD70, PSMA, PSCA, EGFRvIII, claudin6, binds to any pair of CD19/CD79b, BCMA/TACI, or is a TriPRIL antigen binding domain. In some embodiments, the extracellular target binding domain binds to CD19. In some embodiments, the extracellular target binding domain is not derived from a human polypeptide sequence. In some embodiments, the extracellular target binding domain is derived from a murine polypeptide sequence.

In some embodiments, the transmembrane domain is selected from the group consisting of alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), 4-1BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD11 d, ITGAE, CD103, ITGAL, CD11 a, LFA-1, ITGAM, CD11b, ITGAX, CD11c,ITGB1, CD29, ITGB2, CD18, LFA-1, ITGB7, TNFR2, DNAM1 (CD226), SLAMF4 (CD244, 2B4), CD84, CD96 (Tactile), CEACAMI, CRT AM, Ly9 (CD229), CD160 (BY55), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some embodiments, the intracellular signaling domain is selected from the group consisting of CD28, 4-1BB, CD27, TCR-zeta, FcR-gamma, FcR-beta, CD3-gamma, CD3-theta, CD3-sigma, CD3-eta, CD3-epsilon, CD3-zeta, CD22, CD79a, CD79b, and CD66d.

In some embodiments, the polynucleotide comprises a nucleic acid sequence that has at least 85% identity to SEQ ID NO: 17-18. In some embodiments, the polynucleotide comprises a nucleic acid sequence of SEQ ID NO: 19 and a nucleic acid sequence of SEQ ID NO: 20, 22 or 24.

In some embodiments, the polynucleotide is a vector, optionally a lentiviral vector. In some aspects, this application discloses a polynucleotide comprising an shRNA of SEQ ID NO: 13. In some aspects, this application discloses a cell comprising the polynucleotide described herein. In some aspects, the cell comprises the polynucleotide as described herein.

In some aspects, this application discloses a method of modifying the immunogenicity of a cell, the method comprising introducing into the cell an oligonucleotide that is complementary to a polynucleotide encoding an MHC class II complex subunit of any one of SEQ ID NOs: 7-12, wherein the oligonucleotide is selected from the group consisting of a RNA interference (RNAi) oligonucleotide, an antisense oligonucleotide (ASO), or a CRISPR interference (CRISPRi) oligonucleotide. In some aspects, this application discloses a method of decreasing an immune response of a subject to a cell therapy, the method comprising introducing into cells of the cell therapy an oligonucleotide that is complementary to a polynucleotide encoding class II MHC transactivator complex protein of any one of SEQ ID NOs: 7-12, wherein the oligonucleotide is selected from the group consisting of a RNA interference (RNAi) oligonucleotide, an antisense oligonucleotide (ASO), or a CRISPR interference (CRISPRi) oligonucleotide.

In some embodiments, the method further comprises introducing into cells of the cell therapy a virus-derived inhibitor of transporter associated with antigen processing (TAPi) or variant thereof. In some embodiments, the method comprises introducing into cells of the cell therapy the polynucleotide described herein. In some embodiments, the cell or cells are eukaryotic cells. In some embodiments, the cell or cells are immune cells. In some embodiments, the immune cell or immune cells are T cells. In some embodiments, the cells are allogenic to the subject. In some embodiments, the cell therapy is a CAR-T cell therapy. In some embodiments, the CAR-T cell therapy comprises an anti-CD19 CAR-T cell. In some embodiments, the subject is a human subject. In some embodiments, the method decreases natural killer cell activation. In some aspects, this application relates to a method of treating cancer in a subject, the method comprising administering the cell described herein to the subject. In some embodiments, the cancer is a hematological cancer. In some embodiments, the hematological cancer is selected from the group consisting of Leukemia, Lymphoma, and Myeloma. In some embodiments, the hematological cancer is selected from the group consisting of acute lymphoblastic leukemia or mantle cell lymphoma. In some embodiments, the cancer is a solid tumor. In some embodiments, the solid tumor is selected from the group consisting of ovarian cancer, mesothelioma, brain cancer, liver cancer, kidney cancer, lung cancer, breast cancer, prostate cancer, throat cancer, thyroid cancer, colon cancer, testicular cancer, and skin cancer. In some embodiments, the cancer expresses CD19.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1E show that lentivirus transduction of viral TAP inhibitors results in decreased cell surface expression of MHC Class I and allogeneic response in human primary T cells whilst averting an obvious NK cell and pre-existing anti-viral T cell response. FIG. 1A shows the design of the lentiviral constructs expressing viral TAPi or CRISPR-guide RNA for β2M. FIG. 1B shows reduced MHC class I cell surface expression, and, after co-incubation with NK cells, NK cell cytotoxicity and degranulation was found by flow cytometric analysis of TAPi-transduced human primary T cells (n=3 donors). FIG. 1C shows allogeneic and autologous response of T cell against human primary TAPi-expressing T cells or β2M KO T cells was measured by proliferation of responder T cells in an MLR reaction by means of flow cytometric analysis of CellTrace labeled responder cells, which was found to be severely reduced due clearance of MHC I cell surface levels. FIG. 1D shows co-expression of viral TAPi and the HCMV pp65 in primary T cells strongly reduces NLV antigen presentation assessed by IFNγ secretion by NLV-specific CD8+ T cells upon co-incubation measured by IFNγ ELISA. FIG. 1E shows co-incubation of viral TAPi-expressing primary T cells with autologous T cells from donors with a pre-existing anti-viral cellular immunity does not elicit an T cell response as measured by IFNγ ELISPOT. (Asterixes indicated statistical significances compared to the UTD—*: P≤0.05; **: P≤0.01; ***: P≤0.001; ****: P≤0.0001).

FIGS. 2A-2G show lentiviral transduction of shRNA targeting CIITA results in decreased cell surface expression of MHC Class II and allogeneic response in human primary T cells, and can be combined with expression of EBV TAPi to decrease both MHC class I and II, evading allogeneic T cell responses. FIG. 2A shows the design of the lentiviral constructs expressing shRNA targeting CIITA or CRISPR-guide for CIITA. FIGS. 2B-2C show reduced MHC class II cell surface expression was found by flow cytometric analysis of human primary T cells expressing shRNA targeting CIITA whilst T cells transduction with shRNA CIITA3 maintained similar proliferation compared to UTD (n=3 donors). FIG. 2D shows allogeneic and autologous response of T cell against human primary T cells expressing shRNA targeting CIITA was measured by proliferation of responder T cells in an MLR reaction by means of flow cytometric analysis of CellTrace labeled responder cells, which was found to be severely reduced due clearance of MHC II cell surface levels. FIG. 2E is a schematic overview of the different utilized lentiviral constructs combining the EBV TAPi and the shRNA CIITA3. FIG. 2F shows MHC class I and II expression was analyzed in human primary T cells expressing the EBV TAPi and/or shRNA targeting CIITA by flow cytometric analysis (n=3 donors). FIG. 2G shows allogeneic and autologous response of T cells against primary human T cells expressing EBV TAPi and/or shRNA CIITA3 were assessed by responder cell proliferation in an MLR assay. (Asterixes indicated statistical significances compared to the UTD—*: P≤0.05; **: P≤0.01; ***: P≤0.001; ****: P≤0.0001)

FIGS. 3A-3F show the stealth modification to the αCD19 CAR T cells does not alter the tumor-clearing efficacy and CAR T cell proliferation, whilst enabling the stealth αCD19 CAR T cells to evade CAR-mediated immune recognition by T cells from patients who received a single or double infusion of αCD19 CAR T cells. FIG. 3A is a schematic overview of the different lentiviral constructs based on the αCD19 CAR w/o the combination of EBV TAPi and shRNA CIITA3. FIG. 3B shows MHIC class I and II expression, NK cell cytotoxicity after co-incubation with NK cells, and T cell proliferation were analyzed in αCD19 CAR T cells w/o stealth technology. FIG. 3C shows luciferized cytotoxicity assays of αCD19 CAR T cells with or without stealth technology were performed with ALL cell line NALM-6 and Mantle cell line JeKo-1, indicating similar tumor clearance in vitro. FIGS. 3D-3E show NSG mice were engrafted with NALM6 cells and treated with αCD19 CAR T cells with or without stealth technology or left untreated. On day 7 and 14 after treatment, blood was drawn to assess CAR T cell expansion and BLI images were taken to assess the tumor burden. FIG. 3F shows IFNγ ELISpot assays were performed with T cells from patients, who had received the FMC63-based αCD19 CAR T cells (Yescarta or Kymriah) products, and autologous αCD19 CAR T cells with or without stealth technology to assess the CAR-mediated T cell immunity, indicating efficient evasion of CAR-mediated T cell immunity by the stealth technology. (Asterixes indicated statistical significances compared to the UTD—P≤0.05; **: P≤0.01; ***: P≤0.001; ****: P≤0.0001)

FIGS. 4A-4C show eGFP expression in TAPi-expressing primary T cells, and assessment of CD25 and CD69 expression of responder cells in MLR assay assessing allogeneic and autologous responses towards TAPi-expressing T cells. FIG. 4A shows flow cytometric analysis of eGFP expression in TAPi-expressing T cells. FIGS. 4B-4C show flow cytometric analysis of CD25 and CD69 of allogeneic and autologous responder cells in an MLR assay with TAPi-expressing T cells and β2M KO T cells show a reduced immune activation and allogeneic response.

FIGS. 5A-5F show eGFP expression primary T cells expressing shRNA targeting CIITA and/or EBV TAPi and assessment of CD25 and CD69 expression of responder cells in MLR assay assessing allogeneic and autologous responses towards T cells expressing shRNA targeting CIITA and/or EBV TAPi. FIG. 5A shows flow cytometric analysis of eGFP expression in T cells expressing shRNA targeting CIITA. FIGS. 5B-5C show flow cytometric analysis of CD25 and CD69 of allogeneic and autologous responder cells in an MLR assay with T cells expressing shRNA targeting CIITA or CIITA KO T cells show a reduced immune activation and allogeneic response. FIG. 5D shows flow cytometric analysis of eGFP expression in T cells expressing shRNA targeting CIITA and/or the EBV TAPi. FIGS. 5E-5F show flow cytometric analysis of CD25 and CD69 of allogeneic and autologous responder cells in an MLR assay with T cells expressing EBV TAPi and/or shRNA targeting CIITA show a reduced immune activation and allogeneic response.

FIGS. 6A-6E show lentivirus transduction of viral TAP inhibitors results in decreased cell surface expression of MHC Class I and allogeneic response in human primary T cells whilst averting an obvious NK cell and pre-existing anti-viral T cell response. FIG. 6A shows a schematic overview of MHC class I pathway and design of the lentiviral constructs expressing viral TAPi or CRISPR-guide for β2M. FIGS. 6B-6D show reduced MHC class I cell surface expression, and, after co-incubation with NK cells, NK cell cytotoxicity and degranulation was found by flow cytometric analysis of TAPi-transduced human primary T cells (n=3 donors). FIG. 6E shows allogeneic and autologous response of T cell against human primary TAPi-expressing T cells or β2M KO T cells was measured by proliferation of responder T cells in an MLR reaction by means of flow cytometric analysis of CellTrace labeled responder cells, which was found to be severely reduced due clearance of MHC I cell surface levels.

FIGS. 7A-7D show lentiviral transduction of shRNA targeting CIITA results in decreased cell surface expression of MHC Class II and allogeneic response in human primary T cells. FIG. 7A shows a schematic overview of MHC Class II pathway and design of the lentiviral constructs expressing shRNA targeting CIITA or CRISPR-guide for CIITA. FIGS. 7B-7C shows reduced MHC class II cell surface expression was found by flow cytometric analysis of human primary T cells expressing shRNA targeting CIITA whilst T cells transduction with shRNA CIITA3 maintained similar proliferation compared to UTD (n=3 donors). FIG. 7D shows allogeneic and autologous response of T cell against human primary T cells expressing shRNA targeting CIITA was measured by proliferation of responder T cells in an MLR reaction by means of flow cytometric analysis of CellTrace labeled responder cells, which was found to be severely reduced due clearance of MHC II cell surface levels. (Asterixes indicated statistical significances compared to the UTD—*: P≤0.05; **: P≤0.01; ***: P≤0.001; ****: P≤0.0001)

FIGS. 8A-8D show lentiviral transduction of the combination of EBV TAPi and shRNA targeting CIITA decreases both MHC class I and II, evading allogeneic T cell responses. FIG. 8A shows a schematic overview of the different utilized lentiviral constructs combining the EBV TAPi and the shRNA CIITA3. FIGS. 8B-8C show MHC class I and II expression was analyzed in human primary T cells expressing the EBV TAPi and/or shRNA targeting CIITA by flow cytometric analysis (n=3 donors). FIG. 8D shows allogeneic and autologous response of T cells against primary human T cells expressing EBV TAPi and/or shRNA CIITA3 were assessed by responder cell proliferation in an MLR assay. (Asterixes indicated statistical significances compared to the UTD—*: P≤0.05; **: P≤0.01; ***: P≤0.001; ****: P≤0.0001).

FIGS. 9A-9F show the stealth modification to the αCD19 CAR T cells does not alter the in vitro characterization of CAR T cells. FIG. 9A shows a schematic overview of the different lentiviral constructs based on the αCD19 CAR w/o the combination of EBV TAPi and shRNA CIITA3. FIG. 9B shows MHC class I, MHC class II, EBV TAPi and CIITA expression, FIG. 9C shows NK cell cytotoxicity after co-incubation with NK cells, and FIG. 9D shows T cell proliferation were analyzed in αCD19 CAR T cells w/o stealth technology. FIG. 9E shows flowcytometric analysis of CAR-T cell CD4:CD8 ratios and memory phenotypes according to CD45RA and CCR7 expression. FIG. 9F luciferized cytotoxicity assays of αCD19 CAR T cells with or without stealth technology were performed with ALL cell line NALM-6 and Mantle cell line JeKo-1, indicating similar tumor clearance in vitro (Asterixes indicated statistical significances compared to the UTD—*: P≤0.05; **: P≤0.01; ***: P≤0.001; ****: P≤0.0001).

FIGS. 10A-10H shows the stealth modification to the αCD19 CAR T cells does not alter the in vivo tumor-clearing efficacy and CAR T cell proliferation. In FIGS. 10A-10C and 10E-10G, NSG mice were engrafted with NALM6 or JeKo-1 cells and treated with αCD19 CAR T cells with or without stealth technology or left untreated. On day 14 after treatment, blood was drawn to assess CAR T cell expansion and BLI images were taken to assess the tumor burden. FIGS. 10D and 10H show survival as indicated by Kaplain-Meier curve (Asterixes indicated statistical significances compared to the UTD—*: P≤0.05; **: P≤0.01; ***: P≤0.001; ****: P≤0.0001)

FIGS. 11A-11E show the stealth modification to the αCD19 CAR T cells enables the CAR T cells to evade CAR-mediated immune recognition by T cells from patients who received a single or double infusion of αCD19 CAR T cells. IFNγ ELISpot assays were performed with T cells from patients, who had received the FMC63-based αCD19 CAR T cells (Yescarta or Kymriah) products, and autologous αCD19 CAR T cells with or without stealth technology to assess the CAR-mediated T cell immunity, indicating efficient evasion of CAR-mediated T cell immunity by the stealth technology. FIG. 11A shows a swimmer plot of the selected patient population. FIG. 11B shows a schematic overview representing the predicted outcomes of the ELISpot assay. FIGS. 11C-11D show a heatmap and representative wells of the ELISpot assay. FIG. 11E shows histograms depicting eGFP expression levels after sorting and graphs indicating the CAR-mediated T cell activation and anti-CAR responses from the ELISpot assay. (Asterixes indicated statistical significances compared to the UTD—*: P≤0.05; **: P≤0.01; ***: P≤0.001; ****: P≤0.0001).

FIG. 12A-12H show the stealth modification to the αCD19 CAR T cells enables evasion of the in vitro allogeneic response and confers increased CAR T cell proliferation in an allogeneic in vivo model. FIG. 12A shows stealth technology prevents triggering of the allogeneic response after co-incubation with allogeneic T cells as measured by a IFNγ ELIspot and flow-based cytotoxic assay. In FIGS. 12B-12C, NSG mice were engrafted with αCD3/αCD28-expanded allogeneic T cells (UTD ND2), inoculated with NALM6 and treated with αCD19 CAR T cells (ND1) with or without stealth technology or left untreated. On day 14 after treatment, blood was drawn to assess CAR T cell expansion and tumor burden. FIG. 12D shows BLI images taken to assess the tumor burden. In FIG. 12E, NSG mice were engrafted with allogeneic T cells (UTD ND2) pulsed twice with irradiated PBMCs from CAR T cell donor (ND1) and expanded by REP protocol, inoculated with NALM6 and treated with αCD19 CAR T cells (ND1) with or without stealth technology or left untreated. FIG. 12F shows the CAR T cells expansion assessed by weekly blood draws (day 7 to 28) and flow cytometry. In FIG. 12G, the tumor burden was quantified by BLI and total emission was graphed. FIG. 12H shows survival plotted in the Kaplan-Meier curve, indicating the model with REP-expanded allogeneic T cells could be followed longer before triggering severe GVhD. (Asterixes indicated statistical significances compared to the UTD—*: P≤0.05; **: P≤0.01; ***: P≤0.001; ****: P≤0.0001).

FIGS. 13A-13B show an assessment of CD25 and CD69 expression of responder cells in MLR assay assessing allogeneic and autologous responses towards TAPi-expressing T cells. FIG. 13A-13B show flow cytometric analysis of CD25 and CD69 of allogeneic and autologous responder cells in an MLR assay with TAPi-expressing T cells and β2M KO T cells show a reduced immune activation and allogeneic response. (Asterixes indicated statistical significances compared to the UTD—*: P≤0.05; **: P≤0.01; ***: P≤0.001; ****: P≤0.0001).

FIGS. 14A-14B show an assessment of CD25 and CD69 expression of responder cells in MLR assay assessing allogeneic and autologous responses towards T cells expressing shRNA targeting CIITA. FIGS. 14A-14B show flow cytometric analysis of CD25 and CD69 of allogeneic and autologous responder cells in an MLR assay with T cells expressing shRNA targeting CIITA or CIITA KO T cells show a reduced immune activation and allogeneic response. (Asterixes indicated statistical significances compared to the UTD—*: P≤0.05; **: P≤0.01; ***: P≤0.001; ****: P≤0.0001).

FIGS. 15A-15B show an assessment of CD25 and CD69 expression of responder cells in MLR assay assessing allogeneic and autologous responses towards T cells expressing EBV TAPi and shRNA targeting CIITA. FIGS. 15A-15B show flow cytometric analysis of CD25 and CD69 of allogeneic and autologous responder cells in an MLR assay with T cells expressing EBV TAPi and shRNA targeting CIITA show a reduced immune activation and allogeneic response. (Asterixes indicated statistical significances compared to the UTD—P≤0.05; **: P≤0.01; ***: P≤0.001; ****: P≤0.0001).

FIG. 16 shows a survival curve of mice in the allogeneic in vivo model. NSG mice were engrafted with αCD3/αCD28-expanded allogeneic T cells (Allo T cells), inoculated with NALM6 and treated with αCD19 CAR T cells with or without stealth technology or left untreated. Survival was indicated by Kaplan-Meier curve. Mice perished early due to graft-versus-host disease as observed by fur loss and sclerosis.

### DETAILED DESCRIPTION

### Cell Compositions

In some aspects, this application discloses a cell comprising: (i) an inhibitor of transporter associated with antigen processing (TAPi) or variant thereof; and (ii) an oligonucleotide that is complementary to a gene encoding a subunit of MHC class II (e.g. SEQ ID NO: 6) or a gene MHC class II transactivator protein (e.g. (SEQ ID NOs: 7-12), wherein the oligonucleotide is selected from the group consisting of a RNA interference (RNAi) oligonucleotide, an antisense oligonucleotide (ASO), or a CRISPR interference (CRISPRi) oligonucleotide. In some aspects, this application discloses a cell comprising: (i) an inhibitor of transporter associated with antigen processing (TAPi) or variant thereof; and (ii) an oligonucleotide that is complementary to a polynucleotide encoding a MHC class II transactivator protein (e.g. (SEQ ID NOs: 7-12), wherein the oligonucleotide is selected from the group consisting of a RNA interference (RNAi) oligonucleotide, an antisense oligonucleotide (ASO), or a CRISPR interference (CRISPRi) oligonucleotide.

A MHC class II transactivator protein, as described herein, refers to a protein that regulates MHC class II transcription or a protein that is in a protein complex that regulates MHC class II transcription. A MHC class II transactivator protein include, but are not limited to CIITA (SEQ ID NO: 7), RFX (SEQ ID NO: 8), RFXANK (SEQ ID NO: 9), CREB (SEQ ID NO: 10), NFYA (SEQ ID NO: 11), and/or NFYC (SEQ ID NO: 12).

A “variant,” or “variant thereof” as referred to herein, is a sequence (e.g., a polypeptide or polynucleotide) substantially homologous to a native or reference sequence, but which has a sequence different from that of the native or reference polypeptide because of one or a plurality of deletions, insertions, or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

Inhibitor of Transporter Associated with Antigen Processing (TAPi)

A transporter associated with antigen processing (TAP) is a protein that translocates antigenic peptides and participates in loading the angiogenic peptides into MHC class I (e.g., HLA A, B, and C) for antigen presentation to the immune system, e.g., as described in Lehnert, Elisa, and Robert Tamp& Frontiers in immunology (2017): 10., which is incorporated by reference in its entirety. The term “inhibitor of transporter associated with antigen processing (TAPi)” refers to a molecule that inhibits the activity, expression or function of a TAP, e.g. as described in Matschulla et al., Scientific Reports 7.1 (2017): 1-13, which is incorporated by reference in its entirety. In some embodiments, the TAPi inhibits the activity expression or function of MHC class I. In some embodiments, TAPi decreases the expression of the MHC class I in a cell by at least 30% (e.g., at least 30%, at least 40%, at least 50%, at least 60%, at least 70%, at least 80%, at least 90%, at least 95%, at least 98%, or at least 99%). In some embodiments, TAPi decreases the expression of the MHC class I in a cell by 50-90%, 50-95% or 50-99%.

In some embodiments, the TAPi is a viral TAPi. In some embodiments, the TAPi is a Herpesvirus TAPi. In some embodiments, the TAPi is selected from the group consisting of a Herpes Simplex virus (HSV) TAPi, Human Cytomegalovirus (HCMV) TAPi, an Epstein-Barr virus (EBV) TAPi, a varicelloviruses (TAPi), or a poxvirus TAPi, e.g., as described in Matschulla et al., Scientific Reports 7.1 (2017): 1-13. In some embodiments, the TAPi is selected from the group consisting of ICP47 (herpes simplex virus type-1, HSV-1), US6 (human cytomegalovirus, HCMV), BNLF2a (Epstein-Barr virus, EBV), UL49.5 (varicelloviruses), and CPXV12 (poxvirus). In some embodiments, the TAPi is selected from the group consisting of a Herpes Simplex virus (HSV) ICP47 TAPi, Human Cytomegalovirus (HCMV) US6 TAPi, or Epstein-Barr virus (EBV) BNLF2a TAPi. In some embodiments, the TAPi is BNFL2a (EBV). In some embodiments, the TAPi comprises an amino acid sequence that is at least 85% identical (e.g., at least 85% identical, at least 90% identical, at least 95% identical, at least 98% identical, at least 99% identical, at least 99.5% identical, or at least 99.9% identical) to any one of SEQ ID NOs: 1-3. In some embodiments, the TAPi comprises an amino acid sequence selected from the group consisting of SEQ ID NOs: 1-3 or a variant thereof. In some embodiments, the TAPi consists of an amino acid sequence selected from the group consisting of any one of SEQ ID NOs: 1-3.

### Cells Comprising Alternative Methods for Decreasing MHC Class I Expression

In some embodiments, the cell comprises an oligonucleotide (e.g. an RNAi oligonucleotide) that comprises a sequence which is complementary to a TAP. In some embodiments, the cell comprises an oligonucleotide that comprises a sequence which is complementary to a gene encoding a subunit of MHC Class I (e.g., the beta-2-microglobin sequence (SEQ ID NO: 5) or a variant thereof, or the HLA-B sequence (SEQ ID NO: 4) or a variant thereof). In some embodiments, the cell comprises 1, 2, 3, 4, 5, 6, 7, 8, 9, 10, or more oligonucleotides that each comprise a sequence that is complementary to a gene encoding a subunit of MHC class I. In some embodiments, the oligonucleotide is RNAi (e.g., siRNA, miRNA, or shRNA), ASO, or CRISPR sequence (e.g., a CRISPR guide RNA sequence), which are well known in the art and described below.

### Oligonucleotides Complementary to MHC Class II

In some aspects, the cell comprises an oligonucleotide that is complementary to a nucleic acid sequence encoding MHC class II (e.g., HLA DR/DP/DQ) or variants thereof, or a nucleic acid sequence encoding a MHC class II transactivator protein. In some aspects, the cell comprises an oligonucleotide that is complementary to a nucleic acid sequence encoding a MHC class II transactivator protein.

The term “complementary” as described herein refers to the degree of Watson-Crick base pairing between two polynucleotides (e.g. an shRNA and a target mRNA). For example, two polynucleotides may be 90% complementary if 9/10 nucleotides of each of the polynucleotides form a Watson Crick base pair. In some embodiments, complementary may refer to at least 70% (e.g., at least 70%, at least 80%, at least 90%, at least 95%, or at least 99%) of nucleotides in a first polynucleotide Watson-Crick base pairing with a second polynucleotide. In some embodiments, the oligonucleotide may be sufficiently complementary to the target gene to decrease expression of the target gene. The skilled person will understand the oligonucleotide used to decrease gene expression (e.g. RNAi oligonucleotide) may comprise a first sequence that is designed to be complementary to the target gene sequence (e.g. mRNA) and other sequences that are not complementary to the target gene sequence (e.g. sequences for processing). Thus, when an oligonucleotide that is complementary to a gene (e.g. a gene encoding a subunit of MHC class II) is disclosed, the complementarity is referring to the region of oligonucleotide designed to be complementary to the gene.

In some aspects, the cell comprising a TAPi comprises an oligonucleotide that is complementary to a nucleic acid sequence encoding MHC class II (e.g., HLA DR/DP/DQ). In some embodiments, the MHC class II is mammalian MHC class II. In some embodiments, the MHC class II is human MHC class II. In some embodiments, the MHC class II is murine MHC class II.

In some embodiments, the cell comprises an oligonucleotide that is complementary to a gene encoding a MHC class II transactivator protein (e.g., any one of CIITA (SEQ ID NO: 7), RFX (SEQ ID NO: 8), RFXANK (SEQ ID NO: 9), NYFA (SEQ ID NO: 10), NYFC (SEQ ID NO: 11), and NF-gamma and CREB (SEQ ID NO: 12)), or variants thereof. In some aspects, the cell comprises an oligonucleotide that is complementary any one of SEQ ID NOs: 7-12 or a variant thereof. In some embodiments, the cell comprises an oligonucleotide that is complementary to CIITA or a variant thereof. In some aspects, the cell comprises an oligonucleotide that is complementary to SEQ ID NOs: 7 or a variant thereof. In some embodiments, the cell comprises 1, 2, 3, 4, 5, 6, 7, 8, 9, 10, or more oligonucleotides that each comprise a sequence that is complementary to a gene encoding a subunit of MHC class II and/or a MHC class II transactivator protein (e.g., SEQ ID NOs: 7-12). In some embodiments, the cell comprises 1, 2, 3, 4, 5, 6, 7, 8, 9, 10, or more oligonucleotides that each comprise a sequence that is complementary a gene encoding CIITA (SEQ ID NO: 7).

In some embodiments, the oligonucleotide is an RNA interference (RNAi) oligonucleotide, an antisense oligonucleotide (ASO), or a CRISPR interference oligonucleotide. In some embodiments, administration of the oligonucleotide decreases MHC class II expression in the cell.

In some embodiments, the RNAi oligonucleotide is selected from the group consisting of a siRNA, a miRNA, a shRNA or any other suitable RNAi oligonucleotide. Methods of constructing and using siRNAs, miRNAs and shRNA oligonucleotides to decrease the expression of a gene (e.g. MHC class I, MHC class II or a MHC class II transactivator protein) are well known in the art, e.g., as described in Agrawal et al., Microbiology and Molecular Biology Reviews 67.4 (2003): 657-685, and Taxman et al., RNA Therapeutics. Humana Press, 2010. 139-156, both of which are incorporated by reference in their entirety.

In some embodiments, the RNAi oligonucleotide is a shRNA. In some embodiments, the cell comprises an shRNA comprising a sequence that is complementary to any one of SEQ ID NOs: 7-12 or a variant thereof. In some embodiments, the cell comprises an shRNA comprising a sequence that is complementary to a gene encoding CIITA (SEQ ID NO: 7) or a variant thereof. In some embodiments, the shRNA is encoded by a nucleic acid sequence comprising SEQ ID NO: 13 or a variant thereof. In some embodiments, the shRNA is encoded by a nucleic acid sequence comprising SEQ ID NO: 13.

In some embodiments, the oligonucleotide that is complementary to a nucleic acid sequence encoding MHC class II or a MHC class II transactivator protein is an antisense oligonucleotide (ASOs). ASOs are well known in the art as e.g., as described in Quemener, Anais M., et al. Wiley Interdisciplinary Reviews: RNA 11.5 (2020): e1594, which is incorporated by reference in its entirety. In some embodiments, the ASO is a DNA sequence. In some embodiments, the ASO DNA sequence is modified. In some embodiments, the ASO sequence comprises one or more modification selected from the group consisting of phosphorothioate (PS) oligodeoxynucleotides, 2′ methoxyethyl (2′-MOE), 2′ constrained ethyl (2′cEt) modifications, 2′-MOE and 2′cEt PS ASOs conjugated with N-acetyl galactosamine (GalNAc).

In some embodiments, the oligonucleotide that is complementary to a nucleic acid sequence encoding MHC class II or MHC class II transactivator protein is a CRISPR gRNA sequence (e.g., a CRISPR interference guide RNA sequence). Methods of using CRISPR interference and designing CRISPR interference guide RNA sequences are well known in the art as described in Mohr, Stephanie E., et al. The FEBS Journal 283.17 (2016): 3232-3238, which is incorporated by reference in its entirety. In some embodiments, the oligonucleotide that is complementary to a nucleic acid sequence encoding MHC class II or MHC class II transactivator protein is a CRISPR oligonucleotide. In some embodiments, CRISPR may be used to mutate the MHC class II or MHC class II transactivator protein. In some embodiments, the mutation is a loss of function mutation (e.g., a frameshift mutation or early stop codon mutation). In some embodiments, the oligonucleotide that is complementary to a nucleic acid sequence encoding MHC class II or MHC class II transactivator protein is a base editor oligonucleotide. In some embodiments, the base editor is a adenosine base editor or a cytosine base editor. In some embodiments, the base editor mutates gene encoding the MHC class II or MHC class II transactivator protein. In some embodiments, the mutation is a loss of function mutation (e.g., a frameshift mutation or early stop codon mutation).

### Chimeric Antigen Receptor (CAR)

In some embodiments, the cell comprising a TAPi and an oligonucleotide (e.g. RNAi oligonucleotide) that is complementary to a nucleic acid sequence encoding MHC class II transactivator protein (e.g., SEQ ID NOs:7-12) or variants thereof as described herein further comprises a chimeric antigen receptor (CAR).

The terms “chimeric antigen receptor” or “CAR” or “CARs”, as used herein, refer to engineered T cell receptors, which graft a ligand or antigen specificity onto T cells (for example, naive T cells, central memory T cells, effector memory T cells or combinations thereof). CARs are also known as artificial T-cell receptors, chimeric T-cell receptors or chimeric immunoreceptors.

A CAR places a chimeric extracellular antigen-binding domain that specifically binds a target, e.g., a polypeptide, expressed on the surface of a cell to be targeted for an immune cell response (e.g., a T cell) onto a construct including a transmembrane domain and intracellular domain(s) of a T cell receptor molecule. In some embodiments, the chimeric extracellular antigen-binding domain includes the antigen-binding domain(s) of an antibody reagent that specifically binds an antigen expressed on a cell to be targeted for a T cell response. In some embodiments, the chimeric extracellular antigen-binding domain includes a ligand that specifically binds an antigen expressed on a cell to be targeted for a T cell response.

As used herein, a “CART cell”, “CAR-T cell”, or “CAR T cell” refers to a T cell that expresses a CAR. When expressed in a T cell, CARs have the ability to redirect T-cell specificity and reactivity toward a selected target in a non-MHC-restricted manner, exploiting the antigen-binding properties of monoclonal antibodies. The non-MHC-restricted antigen recognition gives T-cells expressing CARs the ability to recognize an antigen independent of antigen processing, thus bypassing a major mechanism of tumor escape.

In some embodiments, the CAR polypeptide comprises an amino acid sequence with at least 75%, at least 80%, at least 85%, at least 90%, at least 91%, at least 92%, at least 93%, at least 94%, at least 95%, at least 96%, at least 97%, at least 98%, at least 99% or greater sequence identity to SEQ ID NO: 17. In some embodiments, the CAR polypeptide comprises an amino acid sequence of any one of SEQ ID NO: 17. In some embodiments, the CAR polypeptide consists of an amino acid sequence of any one of SEQ ID NO: 17. As can be determined by those of skill in the art, various functionally similar or equivalent components of these CARs can be swapped or substituted with one another, as well as other similar or functionally equivalent components known in the art or listed herein.

### Extracellular Antigen-Binding Domain

As used herein, the term “extracellular antigen-binding domain” refers to a polypeptide found on the outside of the cell that is sufficient to facilitate binding to a target. The extracellular target binding domain will specifically bind to its binding partner, i.e., the target. As non-limiting examples, the extracellular antigen-binding domain can include an antigen-binding domain of an antibody or antibody reagent, or a ligand, which recognizes and binds with a cognate binding partner protein. In this context, a ligand is a molecule that binds specifically to a portion of a protein and/or receptor. The cognate binding partner of a ligand useful in the methods and compositions described herein can generally be found on the surface of a cell. Ligand:cognate partner binding can result in the alteration of the ligand-bearing receptor, or activate a physiological response, for example, the activation of a signaling pathway. In some embodiments, the ligand can be non-native to the genome. In some embodiments, the ligand has a conserved function across at least two species.

Any cell-surface moiety can be targeted by a CAR. Often, the target will be a cell-surface polypeptide that may be differentially or preferentially expressed on a cell that one wishes to target for a T cell response. In some embodiments, the extracellular target binding domain binds to any one of CD19, CD37, CD70, CD79b, TACI, BCMA, MUC1, MUC16, B7H3, mesothelin, CD70, PSMA, PSCA, EGFRvIII, claudin6, binds to any pair of CD19/CD79b, BCMA/TACI, or is a TriPRIL antigen binding domain, e.g., as described in PCT/US2020/065733, PCT/US2020/036108, PCT/US2018/013215, PCT/US2018/013213, PCT/US2018/027783, PCT/US2018/013221, PCT/US2018/022974, PCT/US2019/042268, PCT/US2019/038518, PCT/US2019/066357, PCT/US2019/013103, PCT/US2019/017727, PCT/US2020/051018, and/or PCT/US2018/013095, each of which are incorporated by reference in its entirety. In some embodiments, the extracellular target binding domain is not human. In some embodiments, the extracellular target binding domain is murine. In some embodiments, the extracellular target binding domain binds to CD19. In some embodiments, the CD19 antibody is FMC63 (VH: SEQ ID NO: 39 or VL: SEQ ID NO: 40) or a variant thereof. In some embodiments, the extracellular target binding domain comprises a VH amino acid sequence that has at least 85% identify to SEQ ID NO: 39 and a VL amino acid sequence that has at least 85% identify to SEQ ID NO: 40.

In various embodiments, the CARs described herein include an antibody reagent or an antigen-binding domain thereof as an extracellular target-binding domain. As used herein, the term “antibody reagent” refers to a polypeptide that includes at least one immunoglobulin variable domain or immunoglobulin variable domain sequence and which specifically binds a given antigen. In some embodiments, an antibody reagent can include an antibody or a polypeptide including an antigen-binding domain of an antibody. In some embodiments of any of the aspects, an antibody reagent can include a monoclonal antibody or a polypeptide including an antigen-binding domain of a monoclonal antibody. For example, an antibody can include a heavy (H) chain variable region (abbreviated herein as VH), and a light (L) chain variable region (abbreviated herein as VL). In some embodiments, an antibody includes two heavy (H) chain variable regions and two light (L) chain variable regions. In some embodiments, the antibody reagent is a bispecific antibody reagent.

The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g., de Wildt et al., Eur. J. Immunol. 26(3):629-639, 1996; which is incorporated by reference herein in its entirety)) as well as complete antibodies. An antibody can have the structural features of lgA, lgG, lgE, lgD, or lgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like. In some embodiments, the CAR comprises an antibody reagent. In some embodiments, the therapeutic agent comprises an antibody reagent.

Fully human antibody binding domains can be selected, for example, from phage display libraries using methods known to those of ordinary skill in the art. Furthermore, antibody reagents include single domain antibodies, such as camelid antibodies.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A. et al. (1991) Sequences of Proteins of Immunological Interest, Fifth Edition, U.S. Department of Health and Human Services, NIH Publication No. 91-3242, and Chothia et al., J. Mol. Biol. 196:901-917, 1987; each of which is incorporated by reference herein in its entirety). Each VH and VL is typically composed of three CDRs and four FRs, arranged from amino-terminus to carboxy-terminus in the following order: FR1, CDR1, FR2, CDR2, FR3, CDR3, FR4.

In some embodiments, the antibody or antibody reagent is not a human antibody or antibody reagent (i.e., the antibody or antibody reagent is mouse), but has been humanized. A “humanized antibody or antibody reagent” refers to a non-human antibody or antibody reagent that has been modified at the protein sequence level to increase its similarity to antibody or antibody reagent variants produced naturally in humans. One approach to humanizing antibodies employs the grafting of murine or other non-human CDRs onto human antibody frameworks.

In some embodiments, the extracellular target binding domain of a CAR includes or consists essentially of a single-chain Fv (scFv) fragment created by fusing the VH and VL domains of an antibody, generally a monoclonal antibody, via a flexible linker peptide. In various embodiments, the scFv is fused to a transmembrane domain and to a T cell receptor intracellular signaling domain, e.g., an engineered intracellular signaling domain as described herein. In another embodiment, the extracellular target binding domain of a CAR includes a camelid antibody.

In some embodiments, the antibody reagent binds to a tumor associated-antigen. Non-limiting examples of additional tumor antigens, tumor-associated antigens, or other antigen of interest include activated fibroblast marker, CD19, CD37, BCMA (tumor necrosis factor receptor superfamily member 17 (TNFRSF17); NCBI Gene ID: 608; NCBI Ref Seq: NP 001183.2 and mRNA (e.g., NCBI Ref Seq: NM_001192.2)), CEA, immature laminin receptor, TAG-72, HPV E6 and E7, BING-4, calcium-activated chloride channel 2, cyclin B1, 9D7, Ep-CAM, EphA3, 15 her2/neu, telomerase, EGFR, EGFRviii SAP-1, 2lurviving, BAGE family, CAGE family, GAGE family, MAGE family, SAGE family, XAGE family, NY-ESO-1/LAGE-1, PRAME, SSX-2, Melan-NMART-1, gp100/pmel17, tyrosinase, TRP-1/-2, MC1R, BRCA1/2, CDK4, MART-2, p53, Ras, MUC1, TGF-BetaRII, IL-15, IL-13Ra2, and CSF1 R. In some embodiments, the activated fibroblast marker comprises any one of αSMA (ACTA2), fibroblast activation protein (FAP), platelet derived growth factor receptor-α and -β (PDGFRA, PDGFRB), fibroblast specific protein 1 (FSP1/S100A4), endoglin (ENG), transgelin (TAGLN), tenascin C (TNC), periostin (POSTN), chondroitin sulphate proteoglycan 4 or neuron-glial antigen 2 (CSPG4/NG2), podoplanin (PDPN), or osteopontin (SPP1).

### Hinge and Transmembrane Domains

Each CAR as described herein includes a transmembrane domain, e.g., a hinge/transmembrane domain, which joins the extracellular antigen-binding domain to the intracellular signaling domain. The binding domain of the CAR is optionally followed by one or more “hinge domains,” which plays a role in positioning the antigen binding domain away from the effector cell surface to enable proper cell/cell contact, antigen binding and activation. A CAR optionally includes one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs described herein include the hinge region derived from the extracellular regions of type 1 membrane proteins such as CD8 (e.g., CD8alpha), CD4, CD28, 4-1BB, and CD7, which may be wild-type hinge regions from these molecules or may be altered.

In some embodiments, the hinge region is derived from the hinge region of an immunoglobulin-like protein (e.g., IgA, IgD, IgE, IgG, or IgM), CD28, or CD8. In some embodiments, the hinge domain includes a CD8a hinge region.

As used herein, “transmembrane domain” (TM domain) refers to the portion of the CAR that fuses the extracellular binding portion, optionally via a hinge domain, to the intracellular portion (e.g., the costimulatory domain and intracellular signaling domain) and anchors the CAR to the plasma membrane of the immune effector cell. The transmembrane domain is a generally hydrophobic region of the CAR, which crosses the plasma membrane of a cell. The TM domain can be the transmembrane region or fragment thereof of a transmembrane protein (for example a Type I transmembrane protein or other transmembrane protein), an artificial hydrophobic sequence, or a combination thereof. While specific examples are provided herein and used in the Examples, other transmembrane domains will be apparent to those of skill in the art and can be used in connection with alternate embodiments of the technology. A selected transmembrane region or fragment thereof would preferably not interfere with the intended function of the CAR.

As used in relation to a transmembrane domain of a protein or polypeptide, “fragment thereof” refers to a portion of a transmembrane domain that is sufficient to anchor or attach a protein to a cell surface.

In some embodiments, the transmembrane domain or fragment thereof of the CAR described herein includes a transmembrane domain selected from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), 4-1BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD11 d, ITGAE, CD103, ITGAL, CD11 a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Lyl08), SLAM (SLAMFI, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain is CD8 transmembrane domain.

As used herein, a “hinge/transmembrane domain” refers to a domain including both a hinge domain and a transmembrane domain. For example, a hinge/transmembrane domain can be derived from the hinge/transmembrane domain of CD8, CD28, CD7, or 4-1BB. In some embodiments, the hinge/transmembrane domain of a CAR or fragment thereof is derived from or includes the hinge/transmembrane domain of CD8. CD8 is an antigen preferentially found on the cell surface of cytotoxic T lymphocytes. CD8 mediates cell-cell interactions within the immune system, and acts as a T cell co-receptor. CD8 consists of an alpha (CD8alpha or CD8a) and beta (CD813 or CD8b) chain. CD8a sequences are known for a number of species, e.g., human CD8a, (NCBI Gene ID: 925) polypeptide (e.g., NCBI Ref Seq NP 001139345.1) and mRNA (e.g., NCBI Ref Seq NM_000002.12). CD8 can refer to human CD8, including naturally occurring variants, molecules, and alleles thereof. In some embodiments of any of the aspects, e.g., in veterinary applications, CD8 can refer to the CD8 of, e.g., dog, cat, cow, horse, pig, and the like.

Homologs and/or orthologs of human CD8 are readily identified for such species by one of skill in the art, e.g., using the NCBI ortholog search function or searching available sequence data for a given species for sequence similar to a reference CD8 sequence.

In some embodiments, the hinge and transmembrane sequence corresponds to the amino acid sequence of SEQ ID NO: 25; or includes a sequence with at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 25 or an amino acid sequence having ≤1, ≤2, ≤3, ≤4, ≤5, ≤6, ≤7, ≤8, ≤9, or ≤10 substitutions relative to any thereof.

### Co-Stimulatory Domains

Each CAR described herein optionally includes the intracellular domain of one or more co-stimulatory molecule or co-stimulatory domain. As used herein, the term “co-stimulatory domain” refers to an intracellular signaling domain of a co-stimulatory molecule. Co-stimulatory molecules are cell surface molecules other than antigen receptors or Fe receptors that provide a second signal required for efficient activation and function of T lymphocytes upon binding to antigen. The co-stimulatory domain can be, for example, the co-stimulatory domain of 4-1BB, CD27, CD28, or OX40. In one example, a 4-1BB intracellular domain (ICD) can be used (see, e.g., below and SEQ ID NO: 26, or variants thereof). Additional illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP1O, LAT, NKD2C SLP76, TRIM, and ZAP70. In some embodiments, the intracellular domain is the intracellular domain of 4-1 BB. 4-1 BB (CD137; TNFRS9) is an activation induced costimulatory molecule, and is an important regulator of immune responses.

4-1BB is a membrane receptor protein, also known as CD137, which is a member of the tumor necrosis factor (TNF) receptor superfamily. 4-1BB is expressed on activated T lymphocytes. 4-1BB sequences are known for a number of species, e.g., human 4-1 BB, also known as TNFRSF9 (NCBI Gene 25 ID: 3604) and mRNA (NCBI Reference Sequence: NM_001561.5). 4-1BB can refer to human 4-1BB, including naturally occurring variants, molecules, and alleles thereof. In some embodiments of any of the aspects, e.g., in veterinary applications, 4-1BB can refer to the 4-1BB of, e.g., dog, cat, cow, horse, pig, and the like. Homologs and/or orthologs of human 4-1BB are readily identified for such species by one of skill in the art, e.g., using the NCBI ortholog search function or searching available sequence data for a given species for sequence similar to a reference 4-1 BB sequence.

In some embodiments, the intracellular domain is the intracellular domain of a 4-1BB. In some embodiments, the 4-1BB intracellular domain corresponds to an amino acid sequence selected from SEQ ID NO: 26; or includes a sequence selected from SEQ ID NO: 26; or includes at least 75%, at least 80%, at least 85%, 35 at least 90%, at least 95%, at least 96%, at least 97%, at least 98%, at least 99%, or at least 100% sequence identity to a sequence selected from SEQ ID NO: 26 or an amino acid sequence having ≤1, ≤2, ≤3, ≤4, ≤5, ≤6, ≤7, ≤8, ≤9, or ≤10 substitutions relative to SEQ ID NO: 26.

### Intracellular Signaling Domains

The properties of the intracellular signaling domain(s) of the CAR can vary as known in the art and as disclosed herein, but the chimeric target/antigen-binding domains(s) render the receptor sensitive to signaling activation when the chimeric target/antigen binding domain binds the target/antigen on the surface of a targeted cell.

With respect to intracellular signaling domains, so-called “first-generation” CARs include those that solely provide CD3-zeta signals upon antigen binding. So-called “second-generation” CARs include those that provide both co-stimulation (e.g., CD28 or CD137) and activation (CD3-zeta;) domains, and so-called “third-generation” CARs include those that provide multiple costimulatory (e.g., CD28 and CD137) domains and activation domains (e.g., CD3-zeta). In various embodiments, the CAR is selected to have high affinity or avidity for the target/antigen—for example, antibody-derived target or antigen binding domains will generally have higher affinity and/or avidity for the target antigen than would a naturally occurring T cell receptor. This property, combined with the high specificity one can select for an antibody provides highly specific T cell targeting by CART cells.

CARs as described herein include an intracellular signaling domain. An “intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain. In various examples, the intracellular signaling domain is from CD3-zeta; (see, e.g., below). Additional non-limiting examples of immunoreceptor tyrosine-based activation motif (ITAM)-containing intracellular signaling domains that are of particular use in the technology include those derived from TCR-zeta, FcR-gamma, FcR-beta, CD3-gamma, CD3-theta, CD3-sigma, CD3-eta, CD3-epsilon, CD3-zeta; CD22, CD79a, CD79b, and CD66d.

CD3 is a T cell co-receptor that facilitates T lymphocyte activation when simultaneously engaged with the appropriate co-stimulation (e.g., binding of a co-stimulatory molecule). A CD3 complex consists of 4 distinct chains; mammalian CD3 consists of a CD3-gamma chain, a CD3delta chain, and two CD3-epsilon chains.

These chains associate with a molecule known as the T cell receptor (TCR) and the CD3-zeta to generate an activation signal in T lymphocytes. A complete TCR complex includes a TCR, CD3-zeta, and the complete CD3 complex.

In some embodiments of any aspect, a CAR polypeptide described herein includes an intracellular signaling domain that includes an Immunoreceptor Tyrosine-based Activation Motif or ITAM from CD3-zeta, including variants of CD3-zeta; such as ITAM-mutated CD3-zeta, CD3-eta, or CD3-theta. In some embodiments of any aspect, the ITAM includes three motifs of ITAM of CD3-zeta; (ITAM3). In some embodiments of any aspect, the three motifs of ITAM of CD3-zeta; are not mutated and, therefore, include native or wild-type sequences. In some embodiments, the CD3-zeta; sequence includes the sequence of a CD3-zeta; as set forth in the sequences provided herein, e.g., a CD3-zeta; sequence of SEQ ID NO: 27, or variants thereof.

For example, a CAR polypeptide described herein includes the intracellular signaling domain of CD3-zeta. In some embodiments, the CD3-zeta; intracellular signaling domain corresponds to an amino acid sequence of SEQ ID NO: 27; or includes a sequence selected of SEQ ID NO: 27; or includes a sequence with at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to a sequence of SEQ ID NO: 27 or an amino acid sequence having ≤1, ≤2, ≤3, ≤4, ≤5, ≤6, ≤7, ≤8, ≤9, or ≤10 substitutions relative to SEQ ID NO: 27.

In some embodiments, the intracellular signaling domain comprises a 4-1BB intracellular signaling domain. In some embodiments, the intracellular signaling domain comprises a 4-1BB intracellular signaling domain and a CD3-zeta intracellular signaling domain. In some embodiments, the 4-1BB intracellular signaling domain corresponds to an amino acid sequence of SEQ ID NO: 26; or includes a sequence selected of SEQ ID NO: 26; or includes a sequence with at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to a sequence of SEQ ID NO: 26 or an amino acid sequence having 1, ≤2, ≤3, ≤4, ≤5, ≤6, ≤7, ≤8, ≤9, or 10 substitutions relative to SEQ ID NO: 26.

Individual CAR and other construct components as described herein can be used with one another and swapped in and out of various constructs described herein, as can be determined by those of skill in the art. Each of these components can include or consist of any of the corresponding sequences set forth herein, or variants thereof.

A more detailed description of CARs and CART cells can be found in Maus et al., Blood 123:2624-2635, 2014; Reardon et al., Neuro-Oncology 16:1441-1458, 2014; Hoyos et al., Haematologica 97:1622, 2012; Byrd et al., J. Clin. Oncol. 32:3039-3047, 2014; Maher et al., Cancer Res 69:4559-4562, 2009; and Tamada et al., Clin. Cancer Res. 18:6436-6445, 2012; each of which is incorporated by reference herein in its entirety.

### Signal Peptide

In some embodiments, a CAR polypeptide as described herein includes a signal peptide. Signal peptides can be derived from any protein that has an extracellular domain or is secreted. A CAR polypeptide as described herein may include any signal peptides known in the art. In some embodiments, the CAR polypeptide includes a CD8 signal peptide, e.g., a CD8 signal peptide corresponding to the amino acid sequence of SEQ ID NO: 28, or including the amino acid sequence of SEQ ID NO: 28, or including an amino acid sequence having at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 28 or an amino acid sequence having 1, ≤2, ≤3, ≤4, ≤5, ≤6, ≤7, ≤8, ≤9, or 10 substitutions relative to SEQ ID NO: 28. In some embodiments, the CAR polypeptide includes a IgK signal peptide, e.g., a IgK signal peptide corresponding to the amino acid sequence of SEQ ID NO: 29, or including the amino acid sequence of SEQ ID NO: 29, or including an amino acid sequence having at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 29 or an amino acid sequence having ≤1, ≤2, ≤3, ≤4, ≤5, ≤6, ≤7, ≤8, ≤9, or ≤10 substitutions relative to SEQ ID NO: 29.

In further embodiments, a CAR polypeptide described herein may optionally exclude one of the signal peptides described herein, e.g., a CD8 signal peptide of SEQ ID NO: 28 or an IgK signal peptide of SEQ ID NO: 29.

### Linker Domain

In some embodiments, a CAR further includes a linker domain. As used herein, “linker domain” refers to an oligo- or polypeptide region from about 2 to 100 amino acids in length, which links together any of the domains/regions of the CAR as described herein. In some embodiment, linkers can include or be composed of flexible residues such as glycine and serine so that the adjacent protein domains are free to move relative to one another. Linker sequences useful for the invention can be from 2 to 100 amino acids, 5 to 50 amino acids, 10 to 15 amino acids, 15 to 20 amino acids, or 18 to 20 amino acids in length, and include any suitable linkers known in the art. For instance, linker sequences useful for the invention include, but are not limited to, glycine/serine linkers, e.g., GGGSGGGSGGGS (SEQ ID NO: 31) and Gly4Ser (G4S) (SEQ ID NO: 30) linkers such as (G4S)3 (GGGGSGGGGSGGGGS (SEQ ID NO: 32)) and (G4S)4(GGGGSGGGGSGGGGSGGGGS (SEQ ID NO: 33)); the linker sequence of GSTSGSGKPGSGEGSTKG (SEQ ID NO: 34) as described by Whitlow et al., Protein Eng. 6(8):989-95, 1993, the contents of which are incorporated herein by reference in its entirety; the linker sequence of GGSSRSSSSGGGGSGGGG (SEQ ID NO: 35) as described by Andris-Widhopf et al., Cold Spring Harb. Protoc. 2011 (9), 2011, the contents of which are incorporated herein by reference in its entirety; as well as linker sequences with added functionalities, e.g., an epitope tag or an encoding sequence containing Cre-Lox recombination site as described by Sblattero et al., Nat. Biotechnol. 18(1):75-80, 2000, the contents of which are incorporated herein by reference in its entirety. Longer linkers may be used when it is desirable to ensure that two adjacent domains do not sterically interfere with one another.

Furthermore, linkers may be cleavable or non-cleavable. Examples of cleavable linkers include 2A linkers (e.g., P2A and T2A (SEQ ID NO: 36), 2A-like linkers or functional equivalents thereof and combinations thereof.

For example, a P2A linker sequence can correspond to the amino acid sequence of SEQ ID NO: 37. In various examples, linkers having sequences as set forth herein, or variants thereof, are used. It is to be understood that the indication of a particular linker in a construct in a particular location does not mean that only that linker can be used there. Rather, different linker sequences (e.g., P2A and T2A) can be swapped with one another (e.g., in the context of the constructs of the present invention), as can be determined by those of skill in the art. In some embodiments, the linker region is T2A derived from Thosea asigna virus. Non-limiting examples of linkers that can be used in this technology include T2A, P2A, E2A, BmCPV2A, and BmlFV2A. Linkers such as these can be used in the context of polyproteins, such as those described below. For example, they can be used to separate a CAR component of a polyprotein from a TAPi and/or a oligonucleotide comprising a sequence that is complementary to a gene encoding MHC class II transactivator protein (e.g. an shRNA complementary to CIITA).

### Reporter Molecule

In some embodiments, a CAR as described herein optionally further includes a reporter molecule, e.g., to permit for non-invasive imaging (e.g., positron-emission tomography PET scan). In a bispecific CAR that includes a reporter molecule, the first extracellular binding domain and the second extracellular binding domain can include different or the same reporter molecule. In a bispecific CART cell, the first CAR and the second CAR can express different or the same reporter molecule. In another embodiment, a CAR as described herein further includes a reporter molecule (for example hygromycin phosphotransferase (hph)) that can be imaged alone or in combination with a substrate or chemical (for example 9-[4-[18F]fluoro-3-(hydroxymethyl)butyl]guanine ([18F]FHBG)). In another embodiment, a CAR as described herein further includes nanoparticles at can be readily imaged using non-invasive techniques (e.g., gold nanoparticles (GNP) functionalized with 64Cu2+). Labeling of CART cells for non-invasive imaging is reviewed, for example in Bhatnagar et al., lntegr. Biol. (Camb). 5(1):231-238, 2013, and Keu et al., Sci. Transl. Med. 18; 9(373), 2017, which are incorporated herein by reference in their entireties.

In some embodiments, GFP and mCherry may be used as fluorescent tags for imaging a CAR expressed on a T cell (e.g., a CART cell). It is expected that essentially any fluorescent protein known in the art can be used as a fluorescent tag for this purpose. For clinical applications, the CAR need not include a fluorescent tag or fluorescent protein. In each instance of particular constructs provided herein, therefore, any markers present in the constructs can be removed. The invention includes the constructs with or without the markers. Accordingly, when a specific construct is referenced herein, it can be considered with or without any markers or tags (including, e.g., histidine tags, such as the histidine tag of HIHIHHH (SEQ ID NO: 38)) as being included within the invention.

In some embodiments, the CAR comprises a CD8 leader sequence, an anti-CD19 antibody, a CD8 hinge/transmembrane domain, a 4-1BB intracellular signalling domain, and CD3-zeta intracellular signaling domain and a T2A peptide domain. In some embodiments, the CAR comprises a CD8 leader sequence, a FMC63 heavy chain and light chain, a CD8 hinge/transmembrane domain, a 4-1BB intracellular signaling domain, and CD3-zeta intracellular signaling domain and a T2A peptide domain. In some embodiments, the CAR comprises a CD8 leader sequence, a FMC63 heavy chain, a linker, a FMC63 light chain, a CD8 hinge/transmembrane domain, a 4-1BB intracellular signaling domain, and CD3-zeta intracellular signaling domain and a T2A peptide domain.

### Cell Types

In some embodiments, the cell comprising (i) an inhibitor of transporter associated with antigen processing (TAPi) or variant thereof; and (ii) an oligonucleotide that is complementary to a polynucleotide encoding MHC class II or a MHC class II transactivator protein is a eukaryotic cell. In some embodiments, the cell comprising (i) an inhibitor of transporter associated with antigen processing (TAPi) or variant thereof; and (ii) an oligonucleotide that is complementary to a polynucleotide encoding a MHC class II transactivator protein is a eukaryotic cell. In some embodiments, the cell is a mammalian cell. In some embodiments, the cell is an immune cell. As used herein, “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. In some embodiments, the immune cell is a T cell; a NK cell; a NKT cell; lymphocytes, such as B cells and T cells; and myeloid cells, such as monocytes, macrophages, eosinophils, mast cells, basophils, and granulocytes. In some embodiments, the immune cell is a T cell.

In some embodiments, the immune cell is obtained from a subject having or diagnosed as having cancer, a plasma cell disorder, or autoimmune disease, modified as described herein (e.g. to comprise a TAPi, an anti-MHC class II oligonucleotide, and a CAR), and then administered to the subject.

In some embodiments, the cell is an allogenic cell. The term allogeneic cell refers to a cell that was not derived or extracted from the subject being treated (e.g., the cell is extracted or derived from another). In some embodiments, the allogenic cell is derived from an embryonic stem cell or a induced pluripotent stem cell. In some embodiments, the allogenic cell is extracted from a healthy subject. Because allogenic stem cells are from another, the immunogenicity of the stem cell in the subject may be increased. Thus, introducing the TAPi and/or the anti-MHC class II oligonucleotide may decrease the immunogenicity of the allogenic cell in the subject being treated.

In some embodiments, an immune cell, e.g., a T cell, can be engineered to include any of the TAPi or oligonucleotide complementary to MHC class I, and/or oligonucleotides complementary to MHC class II as described herein. In some embodiments, an immune cell, e.g., a T cell, can be engineered to include any of the CAR polypeptides described herein or known in the art. For example, T cells can be isolated from peripheral blood taken from a donor or patient. T cells can be isolated from a mammal. Preferably, T cells are isolated from a human.

### Polynucleotides Encoding TAPi and an Oligonucleotide.

In some embodiments, this application discloses polynucleotides comprising a first nucleic acid sequence encoding the TAPi and a second nucleic acid sequence encoding an oligonucleotide complementary to MHC Class II (e.g., MHC class II shRNAs), as described herein. In some embodiments, this application discloses polynucleotides comprising a first nucleic acid sequence encoding the TAPi and a second nucleic acid sequence encoding an oligonucleotide complementary to a gene encoding a MHC Class II transactivator protein (e.g. CIITA), as described herein. In some embodiments, the polynucleotide further comprises and a third nucleic acid sequence encoding a CAR as described herein.

In some embodiments, the first nucleic acid sequence, the second nucleic acid sequence, and the third nucleic acid sequence are each operably linked to a promoter. In some embodiments, the first nucleic acid sequence is operably linked to a first promoter and the second nucleic acid sequence is operably linked to a second promoter. In some such embodiments, the third nucleic acid sequence is operably linked to the first promoter, the second promoter, or a third promoter. Promoters can be a constitutively expressed promoter (e.g., an EFla promoter) or an inducibly expressed promoter (e.g., a NFAT promoter). In some embodiments, a promoter is induced by CAR activity or T cell receptor (TCR) activity.

In some embodiments, expression of the TAPi and CAR are driven by the same promoter, e.g., a constitutively expressed promoter (e.g., an EF1 a promoter). In other embodiments, expression of the TAPi and CAR are driven by different promoters. The polynucleotide sequence encoding the CAR can be located upstream of the polynucleotide sequence encoding the TAPi, or the polynucleotide sequence encoding the TAPi can be located upstream of the polynucleotide sequence encoding the CAR. In some embodiments, expression of the oligonucleotide complementary to a gene encoding a MHC Class II transactivator protein (e.g., CIITA) is driven by a different promoter (e.g., a U6 promoter) than expression of the TAPi or the CAR. In some embodiments, the oligonucleotide complementary to a gene encoding a MHC Class II transactivator protein is located upstream of the TAPi and the CAR. In some embodiments, the oligonucleotide complementary a gene encoding a MHC Class II transactivator protein is located downstream of the TAPi and the CAR.

In some embodiments, the nucleic acid sequence encoding the TAPi, the nucleic acid sequence encoding the oligonucleotide complementary a gene encoding a MHC Class II transactivator protein and the nucleic acid sequence encoding a CAR are encoded within the same vector. In some embodiments, the nucleic acid sequence encoding the TAPi, the nucleic acid sequence encoding the oligonucleotide complementary a gene encoding a MHC Class II transactivator protein and the nucleic acid sequence encoding a CAR are encoded on two or three vectors.

In various examples, the vectors are retroviral vectors. Retroviruses, such as lentiviruses, provide a convenient platform for delivery of nucleic acid sequences encoding a gene, or chimeric gene of interest. A selected nucleic acid sequence can be inserted into a vector and packaged in retroviral particles using techniques known in the art. The recombinant virus can then be isolated and delivered to cells, e.g., in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Galster Academic Press (ISBN:978-1-90455-55-4); and Hu and Pathak Pharmacological Reviews 2000 52:493-512; which are incorporated by reference herein in their entirety. Lentiviral system for efficient DNA delivery can be purchased from OriGene; Rockville, MD. In various embodiments, the protein is expressed in the T cell by transfection or electroporation of an expression vector including nucleic acid encoding the protein using vectors and methods that are known in the art. In some embodiments, the vector is a viral vector or a non-viral vector. In some embodiments, the viral vector is a retroviral vector (e.g., a lentiviral vector), an adenovirus vector, or an adeno-associated virus vector.

In some embodiments, the cells (e.g., CAR-T cells) described herein comprises any one of the polynucleotides described above.

Transfection or electroporation methods of vectors and/nucleic acids are known in the art.

Efficient expression of the TAPi and an oligonucleotide comprising a sequence that is complementary to a gene encoding a MHC class II transactivator protein (e.g., an shRNA complementary to CIITA) and/or CAR can be assessed using standard assays that detect the mRNA, DNA, or gene product of the nucleic acid encoding the TAPi, the oligonucleotide and/or CAR (and optional antibody reagent or cytokine), such as RT-PCR, FAGS, northern blotting, western blotting, ELISA, or immunohistochemistry.

### Methods of Use

In some embodiments, this application discloses a method of modifying a cell, the method comprising introducing into the cell an oligonucleotide that is complementary to a polynucleotide encoding MHC class II or a MHC class II transactivator protein (e.g., CIITA, RFX, RFXANK, NYFA, NYFC, NF-gamma and CREB), wherein the oligonucleotide is selected from the group consisting of a RNA interference (RNAi) oligonucleotide, an antisense oligonucleotide (ASO), or a CRISPR interference (CRISPRi) oligonucleotide.

In some embodiments, this application discloses a method of modifying the immunogenicity of a cell, the method comprising introducing into the cell an oligonucleotide that is complementary to a polynucleotide encoding MHC class II or a MHC class II transactivator protein (e.g., CIITA, RFX, RFXANK, NYFA, NYFC, NF-gamma and CREB), wherein the oligonucleotide is selected from the group consisting of a RNA interference (RNAi) oligonucleotide, an antisense oligonucleotide (ASO), or a CRISPR interference (CRISPRi) oligonucleotide. In some embodiments, this application discloses a method of modifying the immunogenicity of a cell, the method comprising introducing into the cell an oligonucleotide that is complementary to a polynucleotide encoding MHC class II transactivator protein CIITA (SEQ ID NO: 7), wherein the oligonucleotide is selected from the group consisting of a RNA interference (RNAi) oligonucleotide, an antisense oligonucleotide (ASO), or a CRISPR interference (CRISPRi) oligonucleotide. In some embodiments, this application discloses a method of modifying the immunogenicity of a cell, the method comprising introducing into the cell an oligonucleotide that is complementary to a polynucleotide encoding MHC class II transactivator protein CIITA (SEQ ID NO: 7), wherein the oligonucleotide is a RNA interference (RNAi) oligonucleotide (e.g., a shRNA).

In some embodiments, this application discloses methods of decreasing a subject's immune response to a cell therapy (e.g., an allogenic cell therapy). In some embodiments, the method comprising introducing (e.g., via electroporation) into cells of the cell therapy, prior to administration of the therapy to a subject, an oligonucleotide that is complementary to a polynucleotide encoding MHC class II or a MHC class II transactivator protein (e.g., CIITA, RFX, RFXANK, NYFA, NYFC, NF-gamma and CREB), wherein the oligonucleotide is selected from the group consisting of a RNA interference (RNAi) oligonucleotide, an antisense oligonucleotide (ASO), or a CRISPR interference (CRISPRi) oligonucleotide. In some embodiments, introducing the oligonucleotide comprises introducing a vector encoding the oligonucleotide (e.g., a vector encoding an shRNA). In some embodiments, introducing the oligonucleotide comprises introducing the oligonucleotide directly into the cell (e.g., transfecting an shRNA). In some embodiments, the oligonucleotide is any one of the oligonucleotides that comprise a sequence that is complementary to MHC class II as described herein. In some embodiments, the oligonucleotide is an shRNA. In some embodiments, the oligonucleotide in complementary to CIITA (SEQ ID NO: 7). In some embodiments, the oligonucleotide comprises a sequence of SEQ ID NO: 8.

In some embodiments, the method further comprises introducing into cells of the cell therapy, prior to administration to the subject, a TAPi or variant thereof, as described herein. In some embodiments, the TAPi is an EBV TAPi (e.g., SEQ ID NO: 3) or a variant thereof.

In some embodiments, the cell therapy is an immune cell therapy as described herein. In some embodiments, the cell therapy is an allogenic cell therapy as described herein. In some embodiments, the cell therapy is an allogenic immune cell therapy as described herein.

In some embodiments, the cell therapy is a CAR-T cell therapy. In some embodiments, the cell therapy is an allogenic CAR-T cell therapy. In some embodiments, the cell therapy comprises any known CAR including any CAR described herein. In some embodiments, the cell therapy comprises an anti-CD19 CAR. In some embodiments, the anti-CD19 CAR comprises an amino acid sequence that is at least 85% identical (e.g., at least 85% identical, at least 90% identical, at least 95% identical, at least 98% identical, at least 99% identical, at least 99.5% identical, or at least 99.9% identical) to SEQ ID NO: 41. In some embodiments, the anti-CD19 CAR comprises an amino acid sequence of SEQ ID NO: 41. In some embodiments, the anti-CD19 CAR comprises an FMC63 VH and VL (e.g., SEQ ID NOs: 39-40), or a variant thereof.

In some embodiments, the subject is a human subject. In some embodiments, the method decreases natural killer cell activation.

In some aspects this application discloses method of treating a subject (e.g., a subject diagnosed with cancer), the method comprising administering a cell therapy comprising a oligonucleotide complementary to a gene encoding a MHC class II transactivator protein (e.g., a shRNA targeting CIITA) and/or a TAPi as described herein. In some aspects this application discloses method of treating a subject (e.g., a subject diagnosed with cancer), the method comprising administering a cell therapy comprising a oligonucleotide complementary to a gene encoding a MHC Class II transactivator protein (e.g., a shRNA targeting CIITA) and a TAPi as described herein. In some aspects this application discloses method of treating a subject (e.g., a subject diagnosed with cancer), the method comprising administering a cell therapy comprising a oligonucleotide complementary to a gene encoding a MHC class II transactivator protein (e.g., a shRNA complementary to CIITA) and a oligonucleotide that is complementary to a subunit of MHC class I (e.g., a shRNA complementary to beta-2-microglobulin).

In some embodiments, the subject is diagnosed with cancer. In some embodiments, the cancer is a hematological cancer. In some embodiments, the hematological cancer is selected from the group consisting of Leukemia, Lymphoma, and Myeloma. In some embodiments, the hematological cancer is selected from the group consisting of Acute lymphoblastic leukemia (ALL), Acute myelogenous leukemia (AML), Chronic myelogenous leukemia (CML), Chronic lymphocytic leukemia (CLL), Hairy cell leukemia, Hodgkin's disease, Non-Hodgkin lymphoma (many subtypes), Chronic lympocytic leukemia, Follicular Lymphoma, Marginal zone lymphoma, diffuse large B-cell lymphoma, mantle cell lymphoma, and Multiple myeloma. In some embodiments, the hematological cancer is selected from the group consisting of acute lymphoblastic leukemia or mantle cell lymphoma. In some embodiments, the hematological cancer is B-cell acute lymphoblastic leukemia (B-ALL), acute lymphoblastic leukemia/lymphoma (ALL/LBL), or B cell lymphoma.

In some embodiments, the cancer is a solid tumor. In some embodiments, the solid tumor cancer is selected from the group consisting of ovarian cancer, mesothelioma, brain cancer, liver cancer, kidney cancer, lung cancer, breast cancer, prostate cancer, throat cancer, thyroid cancer, colon cancer, testicular cancer, and skin cancer.

In some embodiments, the cancer is characterized by cells that express CD19.

### Subject

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient,” and “subject” are used interchangeably herein. Preferably, the subject is a mammal. The mammal can be a human, non-human primate, mouse, rat, dog, cat, horse, or cow, but is not limited to these examples. Mammals other than humans can be advantageously used as subjects that represent animal models of disease, e.g., cancer. A subject can be male or female.

A subject can be one who has been previously diagnosed with or identified as suffering from or having a condition in need of treatment (e.g., a pancreatic cancer, a lung cancer, an ovarian cancer, endometrial cancer, biliary cancer, gastric cancer, or mesothelioma or another type of cancer expressing mesothelin, among others) or one or more complications related to such a condition, and optionally, have already undergone treatment for the condition or the one or more complications related to the condition.

Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

### Pharmaceutical Compositions

As used herein, the term “pharmaceutical composition” refers to an active agent (e.g., a cell therapy as described herein) in combination with a pharmaceutically acceptable carrier e.g., a carrier commonly used in the pharmaceutical industry.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier in

which the active ingredient would not be found to occur in nature.

In one aspect of the technology, the technology described herein relates to a pharmaceutical composition including activated CART cells comprising a TAPi and a oligonucleotide comprising a sequence that is complementary to a gene encoding a MHC class II transactivator protein (e.g., an shRNA complementary to CIITA) as described herein, and optionally a pharmaceutically acceptable carrier. The active ingredients of the pharmaceutical composition at a minimum include activated CART cells (e.g., comprising a CD19 CAR) comprising a TAPi and a oligonucleotide comprising a sequence that is complementary to a gene encoding a MHC class II transactivator protein (e.g., an shRNA complementary to CIITA) as described herein. In some embodiments, the active ingredients of the pharmaceutical composition consist essentially of activated CART cells (e.g., comprising a CD19 CAR) comprising a TAPi and a oligonucleotide comprising a sequence that is complementary to a gene encoding a MHC class II transactivator protein (e.g., an shRNA complementary to CIITA) as described herein. In some embodiments, the active ingredients of the pharmaceutical composition consist of activated CAR T cells comprising a TAPi and a oligonucleotide comprising a sequence that is complementary to a gene encoding a MHC class II transactivator protein (e.g., an shRNA complementary to CIITA) as described herein. Pharmaceutically acceptable carriers for cell-based therapeutic formulation include saline and aqueous buffer solutions, Ringer's solution, and serum component, such as serum albumin, HDL and LDL. The terms such as “excipient,” “carrier,” “pharmaceutically acceptable carrier”, “pharmaceutically acceptable excipient” or the like are used interchangeably herein.

In some embodiments, the pharmaceutical composition including activated CAR T cells comprising a TAPi and a oligonucleotide comprising a sequence that is complementary to a gene encoding a MHC class II transactivator protein (e.g., an shRNA complementary to CIITA) as described herein can be a parenteral dose form. Since administration of parenteral dosage forms typically bypasses the patient's natural defenses against contaminants, the components apart from the CART cells themselves are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Any of these can be added to the activated CART cells preparation prior to administration. Suitable vehicles that can be used to provide parenteral dosage forms of activated CAR T cells as disclosed within are well known to those skilled in the art. Examples include, without limitation: saline solution; glucose solution; aqueous vehicles including but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Dosage

“Unit dosage form” as the term is used herein refers to a dosage for suitable one administration. By way of example, a unit dosage form can be an amount of therapeutic disposed in a delivery device, e.g., a syringe or intravenous drip bag. In some embodiments, a unit dosage form is administered in a single administration. In another, embodiment more than one unit dosage form can be administered simultaneously.

In some embodiments, the activated CAR T cells comprising a TAPi and a oligonucleotide comprising a sequence that is complementary to a gene encoding a MHC class II transactivator protein (e.g., an shRNA complementary to CIITA) described herein are administered as a monotherapy, i.e., another treatment for the condition is not concurrently administered to the subject. A pharmaceutical composition including the T cells described herein can generally be administered at a dosage of 10⁴ to 10⁹ cells/kg body weight, in some instances 10⁵ to 10⁶ cells/kg body weight, including all integer values within those ranges. If necessary, T cell compositions can also be administered multiple times at these dosages. The cells can be administered by using infusion techniques that are commonly known in immunotherapy (see, e.g., Rosenberg et al., New Eng. J. Med. 30 319:1676, 1988).

In certain aspects, it may be desired to administer activated CART cells comprising a TAPi and a oligonucleotide comprising a sequence that is complementary to a gene encoding a MHC class II transactivator protein (e.g., an shRNA complementary to CIITA) to a subject and then subsequently redraw blood (or have an apheresis performed), activate T cells therefrom as described herein, and reinfuse the patient with these activated and expanded T cells. This process can be carried out multiple times every few weeks. In certain aspects, T cells can be activated from blood draws of from 35 10 cc to 400 cc. In certain aspects, T cells are activated from blood draws of 20 cc, 30 cc, 40 cc, 50 cc, 60cc, 70cc, 80cc, 90cc, or 100cc.

### Administration

In some embodiments, the methods described herein relate to treating a subject having or diagnosed as having cancer, a plasma cell disease or disorder, or an autoimmune disease or disorder with a mammalian cell including any of the CAR polypeptides described herein, or a nucleic acid encoding any of the CAR polypeptides described herein. The CART cells comprising a TAPi and a oligonucleotide comprising a sequence that is complementary to a gene encoding a MHC class II transactivator protein (e.g., an shRNA complementary to CIITA) described herein include mammalian cells including any of the TAPi and a oligonucleotide comprising a sequence that is complementary to a gene encoding a MHC class II transactivator protein (e.g., an shRNA complementary to CIITA) as described herein and any of the CAR polypeptides (and optional antibody reagents or cytokines) described herein or known in the art, or a nucleic acid encoding any of the TAPi and a oligonucleotide comprising a sequence that is complementary to a gene encoding a MHC class II transactivator protein (e.g., an shRNA complementary to CIITA)s or CAR polypeptides described herein.

Subjects having a condition can be identified by a physician using current methods of diagnosing the condition. Symptoms and/or complications of the condition, which characterize these conditions and aid in diagnosis are well known in the art and include but are not limited to, fatigue, persistent infections, and persistent bleeding. Tests that may aid in a diagnosis of, e.g., the condition, but are not limited to, blood screening and bone marrow testing, and are known in the art for a given condition. A family history for a condition, or exposure to risk factors for a condition can also aid in determining if a subject is likely to have the condition or in making a diagnosis of the condition.

The compositions described herein can be administered to a subject having or diagnosed as having a condition. In some embodiments, the methods described herein include administering an effective amount of activated CAR T cells comprising a TAPi and a oligonucleotide comprising a sequence that is complementary to a gene encoding a MHC class II transactivator protein (e.g., an shRNA complementary to CIITA) as described herein to a subject in order to alleviate a symptom of the condition. As used herein, “alleviating a symptom of the condition” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. In some embodiments, the compositions described herein are administered systemically or locally. In a preferred embodiment, the compositions described herein are administered intravenously. In another embodiment, the compositions described herein are administered at the site of a tumor.

The term “effective amount” as used herein refers to the amount of a cell therapy (e.g., activated CAR T cells comprising a TAPi and a oligonucleotide comprising a sequence that is complementary to a gene encoding a MHC class II transactivator protein (e.g., an shRNA complementary to CIITA)) described herein needed to alleviate at least one or more symptom of the disease or disorder, and relates to a sufficient amount of the cell preparation or composition to provide the desired effect. The term “therapeutically effective amount” therefore refers to an amount of a cell therapy described herein that is sufficient to provide a particular anti-condition effect when administered to a typical subject. An effective amount as used herein, in various contexts, would also include an amount sufficient to delay the development of a symptom of the disease, alter the course of a symptom disease (for example but not limited to, slowing the progression of a condition), or reverse a symptom of the condition. Thus, it is not generally practicable to specify an exact “effective amount.” However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a cell therapy (e.g., activated CART cells) comprising a TAPi and a oligonucleotide comprising a sequence that is complementary to a gene encoding a MHIC class II transactivator protein (e.g., an shRNA complementary to CIITA) as described herein, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for bone marrow testing, among others. The dosage can be determined by a physician and adjusted, as necessary, to suit observed effects of the treatment.

### Modes of Administration

Modes of administration of a cell therapy described herein can include, for example intravenous (iv) injection or infusion. The compositions described herein can be administered to a patient transarterially, intratumorally, intranodally, intraperitoneally or intramedullary. In some embodiments, the compositions of T cells may be injected directly into a tumor, lymph node, or site of infection. In some embodiments, the compositions described herein are administered into a body cavity or body fluid (e.g., ascites, pleural fluid, peritoneal fluid, or cerebrospinal fluid).

In some embodiments, subjects may undergo leukapheresis, wherein leukocytes are collected, enriched, or depleted ex vivo to select and/or isolate the cells of interest, e.g., T cells. In some embodiments, the T cells may be extracted from a healthy subject, e.g., via leukapheresis, or differentiated in vitro (e.g., using iPSC or embryonic stem cells). Any of these T cell isolates may be expanded by contact with an artificial APC, e.g., an aAPC expressing anti-CD28 and anti-CD3 CD Rs, and treated such that one or more polynucleotides of the technology (e.g., polynucleotide comprising a TAPi, an oligonucleotide that is complementary to a gene encoding CIITA and a CAR) may be introduced, thereby creating a CAR T cell. Subjects in need thereof can subsequently undergo standard treatment with high dose chemotherapy followed by peripheral blood stem cell transplantation. Following or concurrent with the transplant, subjects can receive an infusion of the expanded CAR T cells. In some embodiment, expanded cells are administered before or following surgery. In some embodiments, lymphodepletion is performed on a subject prior to administering one or more CART cell as described herein. In such embodiments, the lymphodepletion can include administering one or more of melphalan, 40urvivi, cyclophosphamide, and fludarabine. The dosage of the above treatments to be administered to a patient will vary with the precise nature of the condition being treated and the recipient of the treatment. The scaling of dosages for human administration can be performed according to art-accepted practices.

In some embodiments, a single treatment regimen is required. In others, administration of one or more subsequent doses or treatment regimens can be performed. For example, after treatment biweekly for three months, treatment can be repeated once per month, for six months or a year or longer. In some embodiments, no additional treatments are administered following the initial treatment.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Efficacy

The efficacy of the cell therapy (e.g., activated CART cells comprising a TAPi and a oligonucleotide comprising a sequence that is complementary to a gene encoding a MHC class II transactivator protein (e.g., an shRNA complementary to CIITA)) described herein in, e.g., the treatment of a condition described herein, or to induce a response as described herein (e.g., a reduction in cancer cells) can be determined by the skilled clinician. However, a treatment is considered “effective treatment,” as the term is used herein, if one or more of the signs or symptoms of a condition described herein is altered in a beneficial manner, other clinically accepted symptoms are improved, or even ameliorated, or a desired response is induced, e.g., by at least 10% following treatment according to the methods described herein. Efficacy can be assessed, for example, by measuring a marker, indicator, symptom, and/or the incidence of a condition treated according to the methods described herein or any other measurable parameter appropriate.

Treatment according to the methods described herein can reduce levels of a marker or symptom of a condition, e.g., by at least 10%, at least 15%, at least 20%, at least 25%, at least 30%, at least 40%, at least 50%, at least 60%, at least 70%, at least 80% or at least 90% or more.

Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy of a given approach can be assessed in animal models of a condition described herein. When using an experimental animal model, efficacy of treatment is evidenced when a statistically significant change in a marker is observed.

### Cell Therapy

One aspect of the technology described herein relates to a method of treating cancer, a plasma cell disorder, or an autoimmune disease in a subject in need thereof, the method including: engineering a T cell to include any of the TAPi and a oligonucleotide comprising a sequence that is complementary to a gene encoding a MHC Class II protein or a MHC class II transactivator protein (e.g., an shRNA complementary to CIITA) as described herein and include any CAR polypeptides described herein (e.g., a CD19 CAR) or known in the art on the T cell surface; and administering the engineered T cell to the subject. In the case of cancer, the method can be for treating diagnosed cancer, preventing recurrence of cancer, or for use in an adjuvant or neoadjuvant setting. In some embodiments, the method comprises providing a T cell engineered to include any CAR polypeptides described herein or known in the art on the T cell surface; engineering a T cell to include any of the TAPi and a oligonucleotide comprising a sequence that is complementary to a gene encoding a MHC class II transactivator protein (e.g., an shRNA complementary to CIITA) described herein; and administering the engineered T cell to the subject.

One aspect of the technology described herein relates to a method of treating cancer, a plasma cell disorder, or an autoimmune disease in a subject in need thereof, the method including: administering the cell of any of the mammalian cells including any of the TAPi and a oligonucleotide comprising a sequence that is complementary to a gene encoding a MHIC class II transactivator protein (e.g., an shRNA complementary to CIITA) described herein, and any of the CAR polypeptides described herein or known in the art. In some embodiments of any of aspect, the engineered CAR-T cell is stimulated and/or activated prior to administration to the subject.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior technology or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples, which in no way should be construed as being further limiting.

TABLE 1
Sequences

| SEQ ID | | |
|---|---|---|
| NO | Name | Sequence |
| 1 | HSV ICP47 | MSWALEMADTFLDTMRVGPRTYADVRDEINKRGRE |
| | | DREAARTAVHDPERPLLRSPGLLPEIAPNASLGVA |
| | | HRRTGGTVTDSPRNPVTR |
| 2 | HCMV US6 | MDLLIRLGFLLMCALPTPGERSSRDPKTLLSLSPR |
| | | QQACVPRTKSHRPVCYNDTGDCTDADDSWKQLGED |
| | | FAHQCLQAAKKRPKTHKSRPNDRNLEGRLTCQRVR |
| | | RLLPCDLDIHPSHRLLTLMNNCVCDGAVWNAFRLI |
| | | ERHGFFAVTLYLCCGITLLVVILALLCSITYESTG |
| | | RGIRRCGS |
| 3 | EBV BNLF2a | MVHVLERALLEQQSSACGLPGSSTETRPSHPCPED |
| | | PDVSRLRLLLVVLCVLFGLLCLLLI |
| 4 | MHC Class I HLA-B | ATGCTGGTCATGGCGCCCCGAACCGTCCTCCTGCT |
| | *39013 | GCTCTCGGCGGCCCTGGCCCTGACCGAGACCTGGG |
| | | CCGGCTCCCACTCCATGAGGTATTTCTACACCTCC |
| | | GTGTCCCGGCCCGGCCGCGGGGAGCCCCGCTTCAT |
| | | CTCAGTGGGCTACGTGGACGACACGCAGTTCGTGA |
| | | GGTTCGACAGCGACGCCGCGAGTCCGAGAGAGGAG |
| | | CCGCGGGCGCCGTGGATAGAGCAGGAGGGGCCGGA |
| | | ATATTGGGACCGGAACACACAGATCTGCAAGACCA |
| | | ACACACAGACTGACCGAGAGAGCCTGCGGAACCTG |
| | | CGCGGCTACTACAACCAGAGCGAGGCCGGGTCTCA |
| | | CACCCTCCAGAGGATGTACGGCTGCGACGTGGGGC |
| | | CGGACGGGCGCCTCCTCCGCGGGCATAACCAGTTC |
| | | GCCTACGACGGCAAGGATTACATCGCCCTGAACGA |
| | | GGACCTGAGCTCCTGGACCGCGGCGGACACCGCGG |
| | | CTCAGATCACCCAGCGCAAGTGGGAGGCGGCCCGT |
| | | GTGGCGGAGCAGCTGAGAACCTACCTGGAGGGCAC |
| | | GTGCGTGGAGTGGCTCCGCAGATACCTGGAGAACG |
| | | GGAAGGAGACGCTGCAGCGCGCGGACCCCCCAAAG |
| | | ACACATGTGACCCACCACCCCATCTCTGACCATGA |
| | | GGCCACCCTGAGGTGCTGGGCCCTGGGCTTCTACC |
| | | CTGCGGAGATCACACTGACCTGGCAGCGGGATGGC |
| | | GAGGACCAAACTCAGGACACCGAGCTTGTGGAGAC |
| | | CAGACCAGCAGGAGACAGAACCTTCCAGAAGTGGG |
| | | CAGCTGTGGTGGTGCCTTCTGGAGAAGAGCAGAGA |
| | | TACACATGCCATGTACAGCATGAGGGGCTGCCGAA |
| | | GCCCCTCACCCTGAGATGGGAGCCATCTTCCCAGT |
| | | CCACCGTCCCCATCGTGGGCATTGTTGCTGGCCTG |
| | | GCTGTCCTAGCAGTTGTGGTCATCGGAGCTGTGGT |
| | | CGCTGCTGTGATGTGTAGGAGGAAGAGCTCAGGTG |
| | | GAAAAGGAGGGAGCTACTCTCAGGCTGCGTCCAGC |
| | | GACAGTGCCCAGGGCTCTGATGTGTCTCTCACAGC |
| | | TTGA |
| 5 | Beta-2- | ATGTCTCGCTCCGTGGCCTTAGCTGTGCTCGCGCT |
| | microglobulin | ACTCTCTCTTTCTGGCCTGGAGGCTATCCAGCGTA |
| | | CTCCAAAGATTCAGGTTTACTCACGTCATCCAGCA |
| | | GAGAATGGAAAGTCAAATTTCCTGAATTGCTATGT |
| | | GTCTGGGTTTCATCCATCCGACATTGAAGTTGACT |
| | | TACTGAAGAATGGAGAGAGAATTGAAAAAGTGGAG |
| | | CATTCAGACTTGTCTTTCAGCAAGGACTGGTCTTT |
| | | CTATCTCTTGTACTACACTGAATTCACCCCCACTG |
| | | AAAAAGATGAGTATGCCTGCCGTGTGAACCATGTG |
| | | ACTTTGTCACAGCCCAAGATAGTTAAGTGGGATCG |
| | | AGACATGTAA |
| 6 | MHC Class II | ATGATCCTAAACAAAGCTCTGCTGCTGGGGGCCCT |
| | HLA-DQA1*010401 | CGCTCTGACCACCATGATGAGCCCTTGTGGAGGTG |
| | | AAGGCATTGTGGCTGACCACGTTGCCTCTTGTGGT |
| | | GTAAACTTGTACCAGTTTTACGGTCCCTCTGGCCA |
| | | GTACACCCATGAATTTGATGGAGATGAGGAGTTCT |
| | | ACGTGGACCTGGAGAGGAAGGAGACTGCCTGGCGG |
| | | TGGCCTGAGTTCAGCAAATTTGGAGGTTTTGACCC |
| | | GCAGGGTGCACTGAGAAACATGGCTGTGGCAAAAC |
| | | ACAACTTGAACATCATGATTAAACGCTACAACTCT |
| | | ACCGCTGCTACCAATGAGGTTCCTGAGGTCACAGT |
| | | GTTTTCCAAGTCTCCCGTGACACTGGGTCAGCCCA |
| | | ACACCCTCATTTGTCTTGTGGACAACATCTTTCCT |
| | | CCTGTGGTCAACATCACATGGCTGAGCAATGGGCA |
| | | GTCAGTCACAGAAGGTGTTTCTGAGACCAGCTTCC |
| | | TCTCCAAGAGTGATCATTCCTTCTTCAAGATCAGT |
| | | TACCTCACCTTCCTCCCTTCTGCTGATGAGATTTA |
| | | TGACTGCAAGGTGGAGCACTGGGGCCTGGACCAGC |
| | | CTCTTCTGAAACACTGGGAGCCTGAGATTCCAGCC |
| | | CCTATGTCAGAGCTCACAGAGACTGTGGTCTGCAC |
| | | CCTGGGGTTGTCTGTGGGCCTCGTGGGCATTGTGG |
| | | TGGGCACTGTCTTCATCATCCAAGGCCTGCGTTCA |
| | | GTTGGTGCTTCCAGACACCAAGGGCCATTGTGA |
| 7 | CIITA | ATGCGTTGCCTGGCTCCACGCCCTGCTGGGTCCTA |
| | | CCTGTCAGAGCCCCAAGGCAGCTCACAGTGTGCCA |
| | | CCATGGAGTTGGGGCCCCTAGAAGGTGGCTACCTG |
| | | GAGCTTCTTAACAGCGATGCTGACCCCCTGTGCCT |
| | | CTACCACTTCTATGACCAGATGGACCTGGCTGGAG |
| | | AAGAAGAGATTGAGCTCTACTCAGAACCCGACACA |
| | | GACACCATCAACTGCGACCAGTTCAGCAGGCTGTT |
| | | GTGTGACATGGAAGGTGATGAAGAGACCAGGGAGG |
| | | CTTATGCCAATATCGCGGAACTGGACCAGTATGTC |
| | | TTCCAGGACTCCCAGCTGGAGGGCCTGAGCAAGGA |
| | | CATTTTCATAGAGCACATAGGACCAGATGAAGTGA |
| | | TCGGTGAGAGTATGGAGATGCCAGCAGAAGTTGGG |
| | | CAGAAAAGTCAGAAAAGACCCTTCCCAGAGGAGCT |
| | | TCCGGCAGACCTGAAGCACTGGAAGCCAGCTGAGC |
| | | CCCCCACTGTGGTGACTGGCAGTCTCCTAGTGGGA |
| | | CCAGTGAGCGACTGCTCCACCCTGCCCTGCCTGCC |
| | | ACTGCCTGCGCTGTTCAACCAGGAGCCAGCCTCCG |
| | | GCCAGATGCGCCTGGAGAAAACCGACCAGATTCCC |
| | | ATGCCTTTCTCCAGTTCCTCGTTGAGCTGCCTGAA |
| | | TCTCCCTGAGGGACCCATCCAGTTTGTCCCCACCA |
| | | TCTCCACTCTGCCCCATGGGCTCTGGCAAATCTCT |
| | | GAGGCTGGAACAGGGGTCTCCAGTATATTCATCTA |
| | | CCATGGTGAGGTGCCCCAGGCCAGCCAAGTACCCC |
| | | CTCCCAGTGGATTCACTGTCCACGGCCTCCCAACA |
| | | TCTCCAGACCGGCCAGGCTCCACCAGCCCCTTCGC |
| | | TCCATCAGCCACTGACCTGCCCAGCATGCCTGAAC |
| | | CTGCCCTGACCTCCCGAGCAAACATGACAGAGCAC |
| | | AAGACGTCCCCCACCCAATGCCCGGCAGCTGGAGA |
| | | GGTCTCCAACAAGCTTCCAAAATGGCCTGAGCCGG |
| | | TGGAGCAGTTCTACCGCTCACTGCAGGACACGTAT |
| | | GGTGCCGAGCCCGCAGGCCCGGATGGCATCCTAGT |
| | | GGAGGTGGATCTGGTGCAGGCCAGGCTGGAGAGGA |
| | | GCAGCAGCAAGAGCCTGGAGCGGGAACTGGCCACC |
| | | CCGGACTGGGCAGAACGGCAGCTGGCCCAAGGAGG |
| | | CCTGGCTGAGGTGCTGTTGGCTGCCAAGGAGCACC |
| | | GGCGGCCGCGTGAGACACGAGTGATTGCTGTGCTG |
| | | GGCAAAGCTGGTCAGGGCAAGAGCTATTGGGCTGG |
| | | GGCAGTGAGCCGGGCCTGGGCTTGTGGCCGGCTTC |
| | | CCCAGTACGACTTTGTCTTCTCTGTCCCCTGCCAT |
| | | TGCTTGAACCGTCCGGGGGATGCCTATGGCCTGCA |
| | | GGATCTGCTCTTCTCCCTGGGCCCACAGCCACTCG |
| | | TGGCGGCCGATGAGGTTTTCAGCCACATCTTGAAG |
| | | AGACCTGACCGCGTTCTGCTCATCCTAGACGGCTT |
| | | CGAGGAGCTGGAAGCGCAAGATGGCTTCCTGCACA |
| | | GCACGTGCGGACCGGCACCGGCGGAGCCCTGCTCC |
| | | CTCCGGGGGCTGCTGGCCGGCCTTTTCCAGAAGAA |
| | | GCTGCTCCGAGGTTGCACCCTCCTCCTCACAGCCC |
| | | GGCCCCGGGGCCGCCTGGTCCAGAGCCTGAGCAAG |
| | | GCCGACGCCCTATTTGAGCTGTCCGGCTTCTCCAT |
| | | GGAGCAGGCCCAGGCATACGTGATGCGCTACTTTG |
| | | AGAGCTCAGGGATGACAGAGCACCAAGACAGAGCC |
| | | CTGACGCTCCTCCGGGACCGGCCACTTCTTCTCAG |
| | | TCACAGCCACAGCCCTACTTTGTGCCGGGCAGTGT |
| | | GCCAGCTCTCAGAGGCCCTGCTGGAGCTTGGGGAG |
| | | GACGCCAAGCTGCCCTCCACGCTCACGGGACTCTA |
| | | TGTCGGCCTGCTGGGCCGTGCAGCCCTCGACAGCC |
| | | CCCCCGGGGCCCTGGCAGAGCTGGCCAAGCTGGCC |
| | | TGGGAGCTGGGCCGCAGACATCAAAGTACCCTACA |
| | | GGAGGACCAGTTCCCATCCGCAGACGTGAGGACCT |
| | | GGGCGATGGCCAAAGGCTTAGTCCAACACCCACCG |
| | | CGGGCCGCAGAGTCCGAGCTGGCCTTCCCCAGCTT |
| | | CCTCCTGCAATGCTTCCTGGGGGCCCTGTGGCTGG |
| | | CTCTGAGTGGCGAAATCAAGGACAAGGAGCTCCCG |
| | | CAGTACCTAGCATTGACCCCAAGGAAGAAGAGGCC |
| | | CTATGACAACTGGCTGGAGGGCGTGCCACGCTTTC |
| | | TGGCTGGGCTGATCTTCCAGCCTCCCGCCCGCTGC |
| | | CTGGGAGCCCTACTCGGGCCATCGGCGGCTGCCTC |
| | | GGTGGACAGGAAGCAGAAGGTGCTTGCGAGGTACC |
| | | TGAAGCGGCTGCAGCCGGGGACACTGCGGGCGCGG |
| | | CAGCTGCTGGAGCTGCTGCACTGCGCCCACGAGGC |
| | | CGAGGAGGCTGGAATTTGGCAGCACGTGGTACAGG |
| | | AGCTCCCCGGCCGCCTCTCTTTTCTGGGCACCCGC |
| | | CTCACGCCTCCTGATGCACATGTACTGGGCAAGGC |
| | | CTTGGAGGCGGCGGGCCAAGACTTCTCCCTGGACC |
| | | TCCGCAGCACTGGCATTTGCCCCTCTGGATTGGGG |
| | | AGCCTCGTGGGACTCAGCTGTGTCACCCGTTTCAG |
| | | GGCTGCCTTGAGCGACACGGTGGCGCTGTGGGAGT |
| | | CCCTGCAGCAGCATGGGGAGACCAAGCTACTTCAG |
| | | GCAGCAGAGGAGAAGTTCACCATCGAGCCTTTCAA |
| | | AGCCAAGTCCCTGAAGGATGTGGAAGACCTGGGAA |
| | | AGCTTGTGCAGACTCAGAGGACGAGAAGTTCCTCG |
| | | GAAGACACAGCTGGGGAGCTCCCTGCTGTTCGGGA |
| | | CCTAAAGAAACTGGAGTTTGCGCTGGGCCCTGTCT |
| | | CAGGCCCCCAGGCTTTCCCCAAACTGGTGCGGATC |
| | | CTCACGGCCTTTTCCTCCCTGCAGCATCTGGACCT |
| | | GGATGCGCTGAGTGAGAACAAGATCGGGGACGAGG |
| | | GTGTCTCGCAGCTCTCAGCCACCTTCCCCCAGCTG |
| | | AAGTCCTTGGAAACCCTCAATCTGTCCCAGAACAA |
| | | CATCACTGACCTGGGTGCCTACAAACTCGCCGAGG |
| | | CCCTGCCTTCGCTCGCTGCATCCCTGCTCAGGCTA |
| | | AGCTTGTACAATAACTGCATCTGCGACGTGGGAGC |
| | | CGAGAGCTTGGCTCGTGTGCTTCCGGACATGGTGT |
| | | CCCTCCGGGTGATGGACGTCCAGTACAACAAGTTC |
| | | ACGGCTGCCGGGGCCCAGCAGCTCGCTGCCAGCCT |
| | | TCGGAGGTGTCCTCATGTGGAGACGCTGGCGATGT |
| | | GGACGCCCACCATCCCATTCAGTGTCCAGGAACAC |
| | | CTGCAACAACAGGATTCACGGATCAGCCTGAGATG |
| | | A |
| 8 | RFX | ATGGCAACACAGGCGTATACTGAGCTACAGGCAGC |
| | | CCCGCCACCATCCCAGCCGCCACAGGCCCCGCCAC |
| | | AAGCCCAGCCCCAGCCGCCACCGCCACCACCCCCA |
| | | GCGGCACCCCAGCCCCCGCAGCCACCCACCGCTGC |
| | | TGCCACCCCTCAGCCCCAATATGTCACCGAGCTGC |
| | | AGAGCCCCCAGCCCCAGGCACAGCCACCGGGTGGC |
| | | CAGAAGCAGTACGTGACGGAGCTCCCGGCTGTACC |
| | | CGCACCCTCGCAGCCAACCGGTGCACCCACCCCTT |
| | | CGCCTGCACCCCAGCAGTACATCGTGGTCACTGTC |
| | | TCTGAAGGTGCCATGCGGGCCAGCGAGACAGTGTC |
| | | GGAGGCCAGCCCCGGCTCCACCGCCAGCCAGACCG |
| | | GCGTTCCTACTCAGGTGGTTCAGCAGGTGCAGGGC |
| | | ACCCAGCAGCGGCTGCTGGTCCAGACGAGCGTGCA |
| | | GGCCAAGCCAGGCCACGTGTCGCCCCTCCAGCTGA |
| | | CCAACATCCAAGTGCCCCAGCAGGCTCTTCCCACG |
| | | CAGCGTCTGGTGGTGCAGAGCGCAGCCCCAGGCAG |
| | | CAAAGGTGGCCAGGTCTCCCTGACGGTCCATGGTA |
| | | CCCAGCAGGTGCACTCGCCCCCAGAGCAGTCGCCG |
| | | GTGCAGGCCAACAGCTCTTCCAGCAAGACAGCCGG |
| | | GGCCCCCACGGGCACAGTGCCACAGCAGCTGCAGG |
| | | TCCACGGCGTCCAGCAGAGTGTCCCCGTCACCCAA |
| | | GAGAGATCTGTGGTCCAGGCCACTCCACAAGCGCC |
| | | CAAACCCGGCCCGGTGCAGCCGCTGACCGTGCAGG |
| | | GCCTCCAGCCAGTCCACGTGGCTCAAGAGGTGCAG |
| | | CAGCTCCAGCAGGTGCCCGTCCCACACGTGTACTC |
| | | CAGCCAGGTGCAGTATGTGGAGGGCGGCGATGCCA |
| | | GCTACACGGCCAGTGCCATCCGTTCCAGCACCTAC |
| | | TCCTATCCCGAGACGCCGCTGTACACGCAGACGGC |
| | | AAGCACCAGCTACTACGAGGCCGCAGGCACGGCCA |
| | | CCCAGGTCAGCACCCCCGCCACCTCCCAGGCGGTG |
| | | GCCAGCAGTGGCTCCATGCCCATGTACGTGTCCGG |
| | | CAGCCAGGTCGTCGCCAGCTCCGCCAGCACTGGGG |
| | | CTGGGGCCAGCAACAGCAGCGGAGGTGGTGGCAGT |
| | | GGTGGTGGCGGCGGCGGCGGGGGAGGCGGTGGCGG |
| | | GGGTGGCAGTGGCAGCACCGGAGGCGGCGGCAGCG |
| | | GAGCAGGCACCTACGTGATCCAAGGCGGCTACATG |
| | | CTGGGCAGTGCCAGCCAGTCTTACTCTCACACCAC |
| | | CCGTGCCTCGCCAGCCACGGTCCAGTGGCTCCTGG |
| | | ACAACTATGAGACGGCTGAGGGCGTGAGTCTGCCA |
| | | CGGAGCACCCTCTACTGCCACTACTTACTGCACTG |
| | | CCAGGAGCAGAAGCTGGAGCCCGTCAACGCCGCCT |
| | | CCTTCGGCAAGCTCATCCGCTCCGTCTTCATGGGC |
| | | CTGCGAACCCGCCGTCTGGGCACCAGGGGCAACTC |
| | | CAAGTACCACTACTATGGCCTGCGCATCAAGGCCA |
| | | GCTCACCCCTGCTGCGGCTGATGGAGGACCAGCAG |
| | | CACATGGCCATGCGGGGCCAGCCCTTCTCGCAGAA |
| | | GCAGAGGCTCAAGCCCATCCAGAAGATGGAAGGCA |
| | | TGACCAACGGCGTGGCGGTGGGGCAGCAGCCGAGC |
| | | ACGGGGCTGTCGGACATCAGCGCCCAGGTGCAGCA |
| | | GTACCAGCAATTTTTGGATGCCTCTCGGAGCCTCC |
| | | CTGACTTCACAGAGCTCGACCTCCAGGGCAAGGTG |
| | | CTGCCTGAGGGCGTCGGGCCCGGGGACATCAAAGC |
| | | CTTCCAGGTCCTGTACCGGGAACACTGTGAGGCCA |
| | | TTGTCGACGTCATGGTGAACCTGCAGTTCACCCTG |
| | | GTGGAGACGCTGTGGAAGACCTTCTGGAGGTACAA |
| | | CCTCAGCCAGCCCAGTGAGGCGCCACCGCTGGCTG |
| | | TACATGACGAGGCCGAGAAGCGACTGCCCAAAGCC |
| | | ATCCTGGTGCTCCTCTCCAAGTTCGAGCCCGTGCT |
| | | CCAATGGACCAAGCACTGTGACAACGTGCTGTACC |
| | | AGGGCCTGGTGGAAATCCTCATTCCCGACGTGCTG |
| | | CGGCCCATCCCCAGTGCCTTGACCCAAGCGATCCG |
| | | GAACTTTGCCAAGAGCCTGGAGAGCTGGCTCACCC |
| | | ACGCCATGGTCAACATCCCCGAGGAGATGCTGCGG |
| | | GTGAAGGTGGCCGCGGCTGGCGCCTTCGCGCAGAC |
| | | ACTGCGGCGCTACACGTCGCTCAACCACCTGGCGC |
| | | AGGCGGCGCGCGCTGTGCTGCAGAACACCGCACAG |
| | | ATCAACCAGATGCTGAGCGACCTCAACCGCGTGGA |
| | | CTTCGCCAACGTGCAGGAGCAGGCCTCGTGGGTGT |
| | | GCCGCTGCGAGGACCGCGTGGTGCAGCGGCTGGAG |
| | | CAGGACTTCAAGGTGACGCTGCAGCAGCAGAACTC |
| | | GCTGGAGCAGTGGGCGGCCTGGCTGGACGGCGTGG |
| | | TGAGCCAGGTGCTCAAGCCCTACCAGGGCAGCGCC |
| | | GGCTTCCCCAAGGCCGCCAAGCTCTTCCTCCTCAA |
| | | GTGGTCCTTCTACAGCTCCATGGTGATCCGGGACC |
| | | TGACCCTGCGCAGCGCCGCCAGCTTCGGTTCCTTC |
| | | CACCTCATCCGGCTGCTCTACGACGAGTACATGTA |
| | | CTACCTGATCGAGCACCGCGTAGCCCAGGCCAAGG |
| | | GCGAGACCCCCATCGCCGTCATGGGCGAGTTCGCC |
| | | AATCTGGCCACCTCCCTGAACCCCCTGGACCCCGA |
| | | CAAAGACGAGGAGGAAGAAGAGGAGGAGGAGAGCG |
| | | AGGACGAGCTGCCGCAGGACATCTCACTGGCGGCT |
| | | GGCGGCGAGTCACCCGCGCTGGGCCCGGAGACCCT |
| | | GGAGCCGCCGGCCAAGCTGGCGCGGACTGACGCGC |
| | | GCGGCCTCTTCGTGCAGGCGCTGCCCTCCAGCTAA |
| 9 | RFXANK | ATGGAGCTTACCCAGCCTGCAGAAGACCTCATCCA |
| | | GACCCAGCAGACCCCTGCCTCAGAACTTGGGGACC |
| | | CTGAAGACCCCGGAGAGGAGGCTGCAGATGGCTCA |
| | | GACACTGTGGTCCTCAGTCTCTTTCCCTGCACCCC |
| | | TGAGCCTGTGAATCCTGAACCGGATGCCAGTGTTT |
| | | CCTCTCCACAGGCAGGCAGCTCCCTGAAGCACTCC |
| | | ACCACTCTCACCAACCGGCAGCGAGGGAACGAGGT |
| | | GTCAGCTCTGCCGGCCACCCTAGACTCCCTGTCCA |
| | | TCCACCAGCTCGCAGCACAGGGGGAGCTGGACCAG |
| | | CTGAAGGAGCATTTGCGGAAAGGTGACAACCTCGT |
| | | CAACAAGCCAGACGAGCGCGGCTTCACCCCCCTCA |
| | | TCTGGGCCTCCGCCTTTGGAGAGATTGAGACCGTT |
| | | CGCTTCCTGCTGGAGTGGGGTGCCGACCCCCACAT |
| | | CCTGGCAAAAGAGCGAGAGAGCGCCCTGTCGCTGG |
| | | CCAGCACAGGCGGCTACACAGACATTGTGGGGCTG |
| | | CTGCTGGAGCGTGACGTGGACATCAACATCTATGA |
| | | TTGGAATGGAGGGACGCCACTGCTGTACGCTGTGC |
| | | GCGGGAACCACGTGAAATGCGTTGAGGCCTTGCTG |
| | | GCCCGAGGCGCTGACCTCACCACCGAAGCCGACTC |
| | | TGGCTACACCCCGATGGACCTTGCCGTGGCCCTGG |
| | | GATACCGGAAAGTGCAACAGGTGATCGAGAACCAC |
| | | ATCCTCAAGCTCTTCCAGAGCAACCTGGTGCCCGC |
| | | TGACCCTGAGTGA |
| 10 | CREB | ATGACCATGGAATCTGGAGCCGAGAACCAGCAGAG |
| | | TGGAGATGCAGCTGTAACAGAAGCTGAAAACCAAC |
| | | AAATGACAGTTCAAGCCCAGCCACAGATTGCCACA |
| | | TTAGCCCAGGTATCTATGCCAGCAGCTCATGCAAC |
| | | ATCATCTGCTCCCACCGTAACTCTAGTACAGCTGC |
| | | CCAATGGGCAGACAGTTCAAGTCCATGGAGTCATT |
| | | CAGGCGGCCCAGCCATCAGTTATTCAGTCTCCACA |
| | | AGTCCAAACAGTTCAGTCTTCCTGTAAGGACTTAA |
| | | AAAGACTTTTCTCCGGAACACAGATTTCAACTATT |
| | | GCAGAAAGTGAAGATTCACAGGAGTCAGTGGATAG |
| | | TGTAACTGATTCCCAAAAGCGAAGGGAAATTCTTT |
| | | CAAGGAGGCCTTCCTACAGGAAAATTTTGAATGAC |
| | | TTATCTTCTGATGCACCAGGAGTGCCAAGGATTGA |
| | | AGAAGAGAAGTCTGAAGAGGAGACTTCAGCACTTC |
| | | CTACACAGCCTGCTGAAGAAGCAGCACGAAAGAGA |
| | | GAGGTCCGTCTAATGGAGAACAGGGAAGCAGCTCG |
| | | AGAGTGTCGTAGAAAGAAGAAAGAATATGTGAAAT |
| | | GTTTAGAAAACAGAGTGGCAGTGCTTGAAAATCAA |
| | | AACAAGACATTGATTGAGGAGCTAAAAGCACTTAA |
| | | GGACCTTTACTGCCACAAATCAGATTAA |
| 11 | NFYA | ATGGAGCAGTATACAGCAAACAGCAATAGTTCGAC |
| | | AGAGCAGATTGTTGTCCAGGCAGGACAGATTCAGC |
| | | AGCAGCAGCAGGGTGGTGTCACTGCTGTGCAGTTG |
| | | CAGACTGAGGCCCAGGTGGCATCCGCCTCAGGCCA |
| | | GCAAGTCCAGACCCTCCAGGTAGTCCAAGGGCAGC |
| | | CATTAATGGTGCAGGTCAGTGGAGGCCAGCTAATC |
| | | ACATCAACTGGCCAACCCATCATGGTCCAGGCTGT |
| | | CCCTGGTGGACAAGGTCAAACCATCATGCAAGTAC |
| | | CTGTTTCTGGAACACAGGGTTTGCAGCAAATACAG |
| | | TTGGTCCCACCTGGACAGATCCAGATCCAGGGTGG |
| | | ACAGGCTGTGCAGGTGCAGGGCCAGCAGGGCCAGA |
| | | CCCAGCAGATCATCATCCAGCAGCCCCAGACGGCT |
| | | GTCACTGCTGGCCAGACTCAGACACAGCAGCAGAT |
| | | TGCTGTCCAGGGACAGCAAGTGGCACAGACTGCTG |
| | | AAGGGCAGACCATCGTCTATCAACCAGTTAATGCA |
| | | GATGGCACCATTCTCCAGCAAGTTACAGTCCCTGT |
| | | TTCAGGCATGATCACTATCCCAGCAGCCAGTTTGG |
| | | CAGGAGCACAGATTGTTCAAACAGGAGCCAATACC |
| | | AACACAACCAGCAGTGGGCAAGGGACTGTCACTGT |
| | | GACACTACCAGTGGCAGGCAATGTGGTCAATTCAG |
| | | GAGGGATGGTCATGATGGTTCCTGGGGCTGGCTCT |
| | | GTGCCTGCTATCCAAAGAATCCCTCTACCTGGAGC |
| | | AGAGATGCTTGAAGAAGAGCCTCTCTACGTGAATG |
| | | CCAAACAATACCACCGTATTCTTAAGAGGAGGCAA |
| | | GCCCGAGCTAAACTAGAGGCAGAAGGGAAAATTCC |
| | | AAAGGAGAGAAGGAAATACCTGCATGAGTCTCGGC |
| | | ACCGTCATGCCATGGCACGGAAGCGTGGTGAAGGT |
| | | GGACGATTTTTCTCTCCAAAGGAAAAGGATAGTCC |
| | | CCATATGCAGGATCCAAACCAAGCCGATGAAGAAG |
| | | CAATGACACAGATCATCCGAGTGTCCTAA |
| 12 | NFYC | ATGTCCACAGAAGGAGGATTTGGTGGTACTAGCAG |
| | | CAGTGATGCCCAGCAAAGCCTACAGTCGTTCTGGC |
| | | CTCGGGTCATGGAAGAAATCCGGAATTTAACAGTG |
| | | AAAGACTTCCGAGTGCAGGAACTCCCACTGGCTCG |
| | | TATTAAGAAGATTATGAAACTGGATGAAGATGTGA |
| | | AGATGATCAGTGCAGAAGCGCCTGTACTCTTTGCC |
| | | AAGGCAGCCCAGATTTTTATCACAGAGTTGACTCT |
| | | TCGAGCCTGGATTCACACAGAAGATAACAAGCGCC |
| | | GGACTCTACAGAGAAATGATATCGCCATGGCAATT |
| | | ACAAAATTTGATCAGTTTGATTTTCTCATCGATAT |
| | | TGTTCCAAGAGATGAACTGAAACCTCCAAAGCGTC |
| | | AGGAGGAGGTGCGCCAGTCTGTAACTCCTGCCGAG |
| | | CCAGTCCAGTACTATTTCACGCTGGCTCAGCAACC |
| | | CACCGCTGTCCAAGTCCAGGGCCAGCAGCAAGGCC |
| | | AGCAGACCACCAGCTCCACGACCACCATCCAGCCT |
| | | GGGCAGATCATCATCGCACAGCCTCAGCAGGGCCA |
| | | GACCACACCTGTGACAATGCAGGTTGGAGAAGGTC |
| | | AGCAGGTGCAGATTGTCCAGGCTCAGCCACAGGGT |
| | | CAAGCCCAACAGGCCCAGAGTGGCACTGGACAGAC |
| | | CATGCAGGTGATGCAGCAGATCATCACTAACACAG |
| | | GAGAGATCCAGCAGATCCCGGTGCAGCTGAATGCC |
| | | GGCCAGCTGCAGTATATCCGCTTAGCCCAGCCTGT |
| | | ATCAGGCACTCAAGTTGTGCAGGGACAGATCCAGA |
| | | CACTTGCCACCAATGCTCAACAGGGGCAAAGAAAT |
| | | GCAAGTCAGGGGAAGCCTCGAAGGTGCCTGAAAGA |
| | | AACCTTACAGATTACACAGACAGAGGTCCAGCAAG |
| | | GACAGCAGCAGTTCAGCCAGTTCACAGATGGACAG |
| | | CAGCTCTACCAGATCCAGCAAGTCACCATGCCTGC |
| | | GGGCCAGGACCTCGCCCAGCCCATGTTCATCCAGT |
| | | CAGCCAACCAGCCCTCCGACGGGCAGGCCCCCCAG |
| | | GTGACCGGCGACTGA |
| 13 | CIITA shRNA | GCTTCTGCTGATGAGATTTATTTTCAAGAGAAATA |
| | | AATCTCATCAGCAGAAGTTTTTT |
| 14 | EBV TAPi Construct | CGTGAGGCTCCGGTGCCCGTCAGTGGGCAGAGCGC |
| | (EF1a + EBV TAPi + | ACATCGCCCACAGTCCCCGAGAAGTTGGGGGGAGG |
| | T2A + eGFP + WHP) | GGTCGGCAATTGAACCGGTGCCTAGAGAAGGTGGC |
| | | GCGGGGTAAACTGGGAAAGTGATGTCGTGTACTGG |
| | | CTCCGCCTTTTTCCCGAGGGTGGGGGAGAACCGTA |
| | | TATAAGTGCAGTAGTCGCCGTGAACGTTCTTTTTC |
| | | GCAACGGGTTTGCCGCCAGAACACAGGTAAGTGCC |
| | | GTGTGTGGTTCCCGCGGGCCTGGCCTCTTTACGGG |
| | | TTATGGCCCTTGCGTGCCTTGAATTACTTCCACCT |
| | | GGCTGCAGTACGTGATTCTTGATCCCGAGCTTCGG |
| | | GTTGGAAGTGGGTGGGAGAGTTCGAGGCCTTGCGC |
| | | TTAAGGAGCCCCTTCGCCTCGTGCTTGAGTTGAGG |
| | | CCTGGCCTGGGCGCTGGGGCCGCCGCGTGCGAATC |
| | | TGGTGGCACCTTCGCGCCTGTCTCGCTGCTTTCGA |
| | | TAAGTCTCTAGCCATTTAAAATTTTTGATGACCTG |
| | | CTGCGACGCTTTTTTTCTGGCAAGATAGTCTTGTA |
| | | AATGCGGGCCAAGATCTGCACACTGGTATTTCGGT |
| | | TTTTGGGGCCGCGGGCGGCGACGGGGCCCGTGCGT |
| | | CCCAGCGCACATGTTCGGCGAGGCGGGGCCTGCGA |
| | | GCGCGGCCACCGAGAATCGGACGGGGGTAGTCTCA |
| | | AGCTGGCCGGCCTGCTCTGGTGCCTGGCCTCGCGC |
| | | CGCCGTGTATCGCCCCGCCCTGGGCGGCAAGGCTG |
| | | GCCCGGTCGGCACCAGTTGCGTGAGCGGAAAGATG |
| | | GCCGCTTCCCGGCCCTGCTGCAGGGAGCTCAAAAT |
| | | GGAGGACGCGGCGCTCGGGAGAGCGGGCGGGTGAG |
| | | TCACCCACACAAAGGAAAAGGGCCTTTCCGTCCTC |
| | | AGCCGTCGCTTCATGTGACTCCACTGAGTACCGGG |
| | | CGCCGTCCAGGCACCTCGATTAGTTCTCGTGCTTT |
| | | TGGAGTACGTCGTCTTTAGGTTGGGGGGAGGGGTT |
| | | TTATGCGATGGAGTTTCCCCACACTGAGTGGGTGG |
| | | AGACTGAAGTTAGGCCAGCTTGGCACTTGATGTAA |
| | | TTCTCCTTGGAATTTGCCCTTTTTGAGTTTGGATC |
| | | TTGGTTCATTCTCAAGCCTCAGACAGTGGTTCAAA |
| | | GTTTTTTTCTTCCATTTCAGGTGTCGTGAAGCTCT |
| | | AGAGCCATGGTACACGTATTGGAACGGGCGCTTTT |
| | | GGAGCAGCAAAGCTCCGCGTGCGGACTCCCAGGTT |
| | | CATCCACGGAAACACGCCCATCTCATCCCTGCCCG |
| | | GAGGACCCCGATGTATCACGACTTAGGCTCTTGCT |
| | | GGTCGTACTTTGCGTGCTCTTTGGACTTCTGTGCC |
| | | TCCTGCTCATCGGCTCAGGAGAGGGCAGAGGAAGT |
| | | CTTCTAACATGCGGTGACGTGGAGGAGAATCCCGG |
| | | CCCTATGGTGAGCAAGGGCGAGGAGCTGTTCACCG |
| | | GGGTGGTGCCCATCCTGGTCGAGCTGGACGGCGAC |
| | | GTAAACGGCCACAAGTTCAGCGTGTCCGGCGAGGG |
| | | CGAGGGCGATGCCACCTACGGCAAGCTGACCCTGA |
| | | AGTTCATCTGCACCACCGGCAAGCTGCCCGTGCCC |
| | | TGGCCCACCCTCGTGACCACCCTGACCTACGGCGT |
| | | GCAGTGCTTCAGCCGCTACCCCGACCACATGAAGC |
| | | AGCACGACTTCTTCAAGTCCGCCATGCCCGAAGGC |
| | | TACGTCCAGGAGCGCACCATCTTCTTCAAGGACGA |
| | | CGGCAACTACAAGACCCGCGCCGAGGTGAAGTTCG |
| | | AGGGCGACACCCTGGTGAACCGCATCGAGCTGAAG |
| | | GGCATCGACTTCAAGGAGGACGGCAACATCCTGGG |
| | | GCACAAGCTGGAGTACAACTACAACAGCCACAACG |
| | | TCTATATCATGGCCGACAAGCAGAAGAACGGCATC |
| | | AAGGTGAACTTCAAGATCCGCCACAACATCGAGGA |
| | | CGGCAGCGTGCAGCTCGCCGACCACTACCAGCAGA |
| | | ACACCCCCATCGGCGACGGCCCCGTGCTGCTGCCC |
| | | GACAACCACTACCTGAGCACCCAGTCCGCCCTGAG |
| | | CAAAGACCCCAACGAGAAGCGCGATCACATGGTCC |
| | | TGCTGGAGTTCGTGACCGCCGCCGGGATCACTCTC |
| | | GGCATGGACGAGCTGTACAAGTAAGTCGACAATCA |
| | | ACCTCTGGATTACAAAATTTGTGAAAGATTGACTG |
| | | GTATTCTTAACTATGTTGCTCCTTTTACGCTATGT |
| | | GGATACGCTGCTTTAATGCCTTTGTATCATGCTAT |
| | | TGCTTCCCGTATGGCTTTCATTTTCTCCTCCTTGT |
| | | ATAAATCCTGGTTGCTGTCTCTTTATGAGGAGTTG |
| | | TGGCCCGTTGTCAGGCAACGTGGCGTGGTGTGCAC |
| | | TGTGTTTGCTGACGCAACCCCCACTGGTTGGGGCA |
| | | TTGCCACCACCTGTCAGCTCCTTTCCGGGACTTTC |
| | | GCTTTCCCCCTCCCTATTGCCACGGCGGAACTCAT |
| | | CGCCGCCTGCCTTGCCCGCTGCTGGACAGGGGCTC |
| | | GGCTGTTGGGCACTGACAATTCCGTGGTGTTGTCG |
| | | GGGAAGCTGACGTCCTTTCCTTGGCTGCTCGCCTG |
| | | TGTTGCCACCTGGATTCTGCGCGGGACGTCCTTCT |
| | | GCTACGTCCCTTCGGCCCTCAATCCAGCGGACCTT |
| | | CCTTCCCGCGGCCTGCTGCCGGCTCTGCGGCCTCT |
| | | TCCGCGTCTTCGCCTTCGCCCTCAGACGAGTCGGA |
| | | TCTCCCTTTGGGCCGCCTCCCCGCCTG |
| 15 | 15 EBV TAPi construct | CGTGAGGCTCCGGTGCCCGTCAGTGGGCAGAGCGC |
| | (EF1a + EBV TAPi + | ACATCGCCCACAGTCCCCGAGAAGTTGGGGGGAGG |
| | WHP) − no eGFP | GGTCGGCAATTGAACCGGTGCCTAGAGAAGGTGGC |
| | | GCGGGGTAAACTGGGAAAGTGATGTCGTGTACTGG |
| | | CTCCGCCTTTTTCCCGAGGGTGGGGGAGAACCGTA |
| | | TATAAGTGCAGTAGTCGCCGTGAACGTTCTTTTTC |
| | | GCAACGGGTTTGCCGCCAGAACACAGGTAAGTGCC |
| | | GTGTGTGGTTCCCGCGGGCCTGGCCTCTTTACGGG |
| | | TTATGGCCCTTGCGTGCCTTGAATTACTTCCACCT |
| | | GGCTGCAGTACGTGATTCTTGATCCCGAGCTTCGG |
| | | GTTGGAAGTGGGTGGGAGAGTTCGAGGCCTTGCGC |
| | | TTAAGGAGCCCCTTCGCCTCGTGCTTGAGTTGAGG |
| | | CCTGGCCTGGGCGCTGGGGCCGCCGCGTGCGAATC |
| | | TGGTGGCACCTTCGCGCCTGTCTCGCTGCTTTCGA |
| | | TAAGTCTCTAGCCATTTAAAATTTTTGATGACCTG |
| | | CTGCGACGCTTTTTTTCTGGCAAGATAGTCTTGTA |
| | | AATGCGGGCCAAGATCTGCACACTGGTATTTCGGT |
| | | TTTTGGGGCCGCGGGCGGCGACGGGGCCCGTGCGT |
| | | CCCAGCGCACATGTTCGGCGAGGCGGGGCCTGCGA |
| | | GCGCGGCCACCGAGAATCGGACGGGGGTAGTCTCA |
| | | AGCTGGCCGGCCTGCTCTGGTGCCTGGCCTCGCGC |
| | | CGCCGTGTATCGCCCCGCCCTGGGCGGCAAGGCTG |
| | | GCCCGGTCGGCACCAGTTGCGTGAGCGGAAAGATG |
| | | GCCGCTTCCCGGCCCTGCTGCAGGGAGCTCAAAAT |
| | | GGAGGACGCGGCGCTCGGGAGAGCGGGCGGGTGAG |
| | | TCACCCACACAAAGGAAAAGGGCCTTTCCGTCCTC |
| | | AGCCGTCGCTTCATGTGACTCCACTGAGTACCGGG |
| | | CGCCGTCCAGGCACCTCGATTAGTTCTCGTGCTTT |
| | | TGGAGTACGTCGTCTTTAGGTTGGGGGGAGGGGTT |
| | | TTATGCGATGGAGTTTCCCCACACTGAGTGGGTGG |
| | | AGACTGAAGTTAGGCCAGCTTGGCACTTGATGTAA |
| | | TTCTCCTTGGAATTTGCCCTTTTTGAGTTTGGATC |
| | | TTGGTTCATTCTCAAGCCTCAGACAGTGGTTCAAA |
| | | GTTTTTTTCTTCCATTTCAGGTGTCGTGAAGCTCT |
| | | AGAGCCATGGTACACGTATTGGAACGGGCGCTTTT |
| | | GGAGCAGCAAAGCTCCGCGTGCGGACTCCCAGGTT |
| | | CATCCACGGAAACACGCCCATCTCATCCCTGCCCG |
| | | GAGGACCCCGATGTATCACGACTTAGGCTCTTGCT |
| | | GGTCGTACTTTGCGTGCTCTTTGGACTTCTGTGCC |
| | | TCCTGCTCATCTAAGTCGACAATCAACCTCTGGAT |
| | | TACAAAATTTGTGAAAGATTGACTGGTATTCTTAA |
| | | CTATGTTGCTCCTTTTACGCTATGTGGATACGCTG |
| | | CTTTAATGCCTTTGTATCATGCTATTGCTTCCCGT |
| | | ATGGCTTTCATTTTCTCCTCCTTGTATAAATCCTG |
| | | GTTGCTGTCTCTTTATGAGGAGTTGTGGCCCGTTG |
| | | TCAGGCAACGTGGCGTGGTGTGCACTGTGTTTGCT |
| | | GACGCAACCCCCACTGGTTGGGGCATTGCCACCAC |
| | | CTGTCAGCTCCTTTCCGGGACTTTCGCTTTCCCCC |
| | | TCCCTATTGCCACGGCGGAACTCATCGCCGCCTGC |
| | | CTTGCCCGCTGCTGGACAGGGGCTCGGCTGTTGGG |
| | | CACTGACAATTCCGTGGTGTTGTCGGGGAAGCTGA |
| | | CGTCCTTTCCTTGGCTGCTCGCCTGTGTTGCCACC |
| | | TGGATTCTGCGCGGGACGTCCTTCTGCTACGTCCC |
| | | TTCGGCCCTCAATCCAGCGGACCTTCCTTCCCGCG |
| | | GCCTGCTGCCGGCTCTGCGGCCTCTTCCGCGTCTT |
| | | CGCCTTCGCCCTCAGACGAGTCGGATCTCCCTTTG |
| | | GGCCGCCTCCCCGCCTG |
| 16 | shRNA targeting | GAGGGCCTATTTCCCATGATTCCTTCATATTTGCA |
| | CIITA construct | TATACGATACAAGGCTGTTAGAGAGATAATTAGAA |
| | (hUS6 + shRNA | TTAATTTGACTGTAAACACAAAGATATTAGTACAA |
| | CIITA3) | AATACGTGACGTAGAAAGTAATAATTTCTTGGGTA |
| | | GTTTGCAGTTTTAAAATTATGTTTTAAAATGGACT |
| | | ATCATATGCTTACCGTAACTTGAAAGTATTTCGAT |
| | | TTCTTGGCTTTATATATCTTGTGGAAAGGACGAAA |
| | | CACCGAACAACAGGATTCACGGATCAGCTTCAAGA |
| | | GAGCTGATCCGTGAATCCTGTTGTTTTTTTT |
| 17 | Anti−CD19 CAR | CGTGAGGCTCCGGTGCCCGTCAGTGGGCAGAGCGC |
| | construct (based on | ACATCGCCCACAGTCCCCGAGAAGTTGGGGGGAGG |
| | FMC63) (EF1a | GGTCGGCAATTGAACCGGTGCCTAGAGAAGGTGGC |
| | aCD19 CAR + T2A | GCGGGGTAAACTGGGAAAGTGATGTCGTGTACTGG |
| | + eGFP + WHP) | CTCCGCCTTTTTCCCGAGGGTGGGGGAGAACCGTA |
| | | TATAAGTGCAGTAGTCGCCGTGAACGTTCTTTTTC |
| | | GCAACGGGTTTGCCGCCAGAACACAGGTAAGTGCC |
| | | GTGTGTGGTTCCCGCGGGCCTGGCCTCTTTACGGG |
| | | TTATGGCCCTTGCGTGCCTTGAATTACTTCCACCT |
| | | GGCTGCAGTACGTGATTCTTGATCCCGAGCTTCGG |
| | | GTTGGAAGTGGGTGGGAGAGTTCGAGGCCTTGCGC |
| | | TTAAGGAGCCCCTTCGCCTCGTGCTTGAGTTGAGG |
| | | CCTGGCCTGGGCGCTGGGGCCGCCGCGTGCGAATC |
| | | TGGTGGCACCTTCGCGCCTGTCTCGCTGCTTTCGA |
| | | TAAGTCTCTAGCCATTTAAAATTTTTGATGACCTG |
| | | CTGCGACGCTTTTTTTCTGGCAAGATAGTCTTGTA |
| | | AATGCGGGCCAAGATCTGCACACTGGTATTTCGGT |
| | | TTTTGGGGCCGCGGGCGGCGACGGGGCCCGTGCGT |
| | | CCCAGCGCACATGTTCGGCGAGGCGGGGCCTGCGA |
| | | GCGCGGCCACCGAGAATCGGACGGGGGTAGTCTCA |
| | | AGCTGGCCGGCCTGCTCTGGTGCCTGGCCTCGCGC |
| | | CGCCGTGTATCGCCCCGCCCTGGGCGGCAAGGCTG |
| | | GCCCGGTCGGCACCAGTTGCGTGAGCGGAAAGATG |
| | | GCCGCTTCCCGGCCCTGCTGCAGGGAGCTCAAAAT |
| | | GGAGGACGCGGCGCTCGGGAGAGCGGGCGGGTGAG |
| | | TCACCCACACAAAGGAAAAGGGCCTTTCCGTCCTC |
| | | AGCCGTCGCTTCATGTGACTCCACTGAGTACCGGG |
| | | CGCCGTCCAGGCACCTCGATTAGTTCTCGTGCTTT |
| | | TGGAGTACGTCGTCTTTAGGTTGGGGGGAGGGGTT |
| | | TTATGCGATGGAGTTTCCCCACACTGAGTGGGTGG |
| | | AGACTGAAGTTAGGCCAGCTTGGCACTTGATGTAA |
| | | TTCTCCTTGGAATTTGCCCTTTTTGAGTTTGGATC |
| | | TTGGTTCATTCTCAAGCCTCAGACAGTGGTTCAAA |
| | | GTTTTTTTCTTCCATTTCAGGTGTCGTGAAGCTCT |
| | | AGAGCCATGGCCCTCCCTGTCACCGCCCTGCTGCT |
| | | TCCGCTGGCTCTTCTGCTCCACGCCGCTCGGCCCG |
| | | AGGTGAAACTGCAGGAGTCAGGACCTGGCCTGGTG |
| | | GCGCCCTCACAGAGCCTGTCCGTCACATGCACTGT |
| | | CTCAGGGGTCTCATTACCCGACTATGGTGTAAGCT |
| | | GGATTCGCCAGCCTCCACGAAAGGGTCTGGAGTGG |
| | | CTGGGAGTAATATGGGGTAGTGAAACCACATACTA |
| | | TAATTCAGCTCTCAAATCCAGACTGACCATCATCA |
| | | AGGACAACTCCAAGAGCCAAGTTTTCTTAAAAATG |
| | | AACAGTCTGCAAACTGATGACACAGCCATTTACTA |
| | | CTGTGCCAAACATTATTACTACGGTGGTAGCTATG |
| | | CTATGGACTACTGGGGTCAAGGAACCTCAGTCACC |
| | | GTCTCCTCAGGTGGAGGTGGCAGCGGAGGAGGTGG |
| | | GTCCGGCGGTGGAGGAAGCGGCGGTGGAGGAAGCG |
| | | ACATCCAGATGACACAGACTACATCCTCCCTGTCT |
| | | GCCTCTCTGGGAGACAGAGTCACCATCAGTTGCAG |
| | | GGCAAGTCAGGACATTAGTAAATATTTAAATTGGT |
| | | ATCAGCAGAAACCAGATGGAACTGTTAAACTCCTG |
| | | ATCTACCATACATCAAGATTACACTCAGGAGTCCC |
| | | ATCAAGGTTCAGTGGCAGTGGGTCTGGAACAGATT |
| | | ATTCTCTCACCATTAGCAACCTGGAGCAAGAAGAT |
| | | ATTGCCACTTACTTTTGCCAACAGGGTAATACGCT |
| | | TCCGTACACGTTCGGAGGGGGGACTAAGTTGGAAA |
| | | TAACACGGGCTGATGCTGCACCAACTGTATCCATC |
| | | TTCCCACCATCCAGTAATACCACTACCCCAGCACC |
| | | GAGGCCACCCACCCCGGCTCCTACCATCGCCTCCC |
| | | AGCCTCTGTCCCTGCGTCCGGAGGCATGTAGACCC |
| | | GCAGCTGGTGGGGCCGTGCATACCCGGGGTCTTGA |
| | | CTTCGCCTGCGATATCTACATTTGGGCCCCTCTGG |
| | | CTGGTACTTGCGGGGTCCTGCTGCTTTCACTCGTG |
| | | ATCACTCTTTACTGTAAGCGCGGTCGGAAGAAGCT |
| | | GCTGTACATCTTTAAGCAACCCTTCATGAGGCCTG |
| | | TGCAGACTACTCAAGAGGAGGACGGCTGTTCATGC |
| | | CGGTTCCCAGAGGAGGAGGAAGGCGGCTGCGAACT |
| | | GCGCGTGAAATTCAGCCGCAGCGCAGATGCTCCAG |
| | | CCTACCAACAGGGGCAGAACCAGCTCTACAACGAA |
| | | CTCAATCTTGGTCGGAGAGAGGAGTACGACGTGCT |
| | | GGACAAGCGGAGAGGACGGGACCCAGAAATGGGCG |
| | | GGAAGCCGCGCAGAAAGAATCCCCAAGAGGGCCTG |
| | | TACAACGAGCTCCAAAAGGATAAGATGGCAGAAGC |
| | | CTATAGCGAGATTGGTATGAAAGGGGAACGCAGAA |
| | | GAGGCAAAGGCCACGACGGACTGTACCAGGGACTC |
| | | AGCACCGCCACCAAGGACACCTATGACGCTCTTCA |
| | | CATGCAGGCCCTGCCGCCTCGGTCCGGAGGCGGCG |
| | | GAGAGGGCAGAGGAAGTCTTCTAACATGCGGTGAC |
| | | GTGGAGGAGAATCCCGGCCCTAGGATGGTGAGCAA |
| | | GGGCGAGGAGCTGTTCACCGGGGTGGTGCCCATCC |
| | | TGGTCGAGCTGGACGGCGACGTAAACGGCCACAAG |
| | | TTCAGCGTGTCCGGCGAGGGCGAGGGCGATGCCAC |
| | | CTACGGCAAGCTGACCCTGAAGTTCATCTGCACCA |
| | | CCGGCAAGCTGCCCGTGCCCTGGCCCACCCTCGTG |
| | | ACCACCCTGACCTACGGCGTGCAGTGCTTCAGCCG |
| | | CTACCCCGACCACATGAAGCAGCACGACTTCTTCA |
| | | AGTCCGCCATGCCCGAAGGCTACGTCCAGGAGCGC |
| | | ACCATCTTCTTCAAGGACGACGGCAACTACAAGAC |
| | | CCGCGCCGAGGTGAAGTTCGAGGGCGACACCCTGG |
| | | TGAACCGCATCGAGCTGAAGGGCATCGACTTCAAG |
| | | GAGGACGGCAACATCCTGGGGCACAAGCTGGAGTA |
| | | CAACTACAACAGCCACAACGTCTATATCATGGCCG |
| | | ACAAGCAGAAGAACGGCATCAAGGTGAACTTCAAG |
| | | ATCCGCCACAACATCGAGGACGGCAGCGTGCAGCT |
| | | CGCCGACCACTACCAGCAGAACACCCCCATCGGCG |
| | | ACGGCCCCGTGCTGCTGCCCGACAACCACTACCTG |
| | | AGCACCCAGTCCGCCCTGAGCAAAGACCCCAACGA |
| | | GAAGCGCGATCACATGGTCCTGCTGGAGTTCGTGA |
| | | CCGCCGCCGGGATCACTCTCGGCATGGACGAGCTG |
| | | TACAAGTAAGTCGACAATCAACCTCTGGATTACAA |
| | | AATTTGTGAAAGATTGACTGGTATTCTTAACTATG |
| | | TTGCTCCTTTTACGCTATGTGGATACGCTGCTTTA |
| | | ATGCCTTTGTATCATGCTATTGCTTCCCGTATGGC |
| | | TTTCATTTTCTCCTCCTTGTATAAATCCTGGTTGC |
| | | TGTCTCTTTATGAGGAGTTGTGGCCCGTTGTCAGG |
| | | CAACGTGGCGTGGTGTGCACTGTGTTTGCTGACGC |
| | | AACCCCCACTGGTTGGGGCATTGCCACCACCTGTC |
| | | AGCTCCTTTCCGGGACTTTCGCTTTCCCCCTCCCT |
| | | ATTGCCACGGCGGAACTCATCGCCGCCTGCCTTGC |
| | | CCGCTGCTGGACAGGGGCTCGGCTGTTGGGCACTG |
| | | ACAATTCCGTGGTGTTGTCGGGGAAGCTGACGTCC |
| | | TTTCCTTGGCTGCTCGCCTGTGTTGCCACCTGGAT |
| | | TCTGCGCGGGACGTCCTTCTGCTACGTCCCTTCGG |
| | | CCCTCAATCCAGCGGACCTTCCTTCCCGCGGCCTG |
| | | CTGCCGGCTCTGCGGCCTCTTCCGCGTCTTCGCCT |
| | | TCGCCCTCAGACGAGTCGGATCTCCCTTTGGGCCG |
| | | CCTCCCCGCCTG |
| 18 | Anti−CD19 CAR | CGTGAGGCTCCGGTGCCCGTCAGTGGGCAGAGCGC |
| | construct (based on | ACATCGCCCACAGTCCCCGAGAAGTTGGGGGGAGG |
| | FMC63) (EF1a | GGTCGGCAATTGAACCGGTGCCTAGAGAAGGTGGC |
| | aCD19 CAR + WHP) | GCGGGGTAAACTGGGAAAGTGATGTCGTGTACTGG |
| | | CTCCGCCTTTTTCCCGAGGGTGGGGGAGAACCGTA |
| | | TATAAGTGCAGTAGTCGCCGTGAACGTTCTTTTTC |
| | | GCAACGGGTTTGCCGCCAGAACACAGGTAAGTGCC |
| | | GTGTGTGGTTCCCGCGGGCCTGGCCTCTTTACGGG |
| | | TTATGGCCCTTGCGTGCCTTGAATTACTTCCACCT |
| | | GGCTGCAGTACGTGATTCTTGATCCCGAGCTTCGG |
| | | GTTGGAAGTGGGTGGGAGAGTTCGAGGCCTTGCGC |
| | | TTAAGGAGCCCCTTCGCCTCGTGCTTGAGTTGAGG |
| | | CCTGGCCTGGGCGCTGGGGCCGCCGCGTGCGAATC |
| | | TGGTGGCACCTTCGCGCCTGTCTCGCTGCTTTCGA |
| | | TAAGTCTCTAGCCATTTAAAATTTTTGATGACCTG |
| | | CTGCGACGCTTTTTTTCTGGCAAGATAGTCTTGTA |
| | | AATGCGGGCCAAGATCTGCACACTGGTATTTCGGT |
| | | TTTTGGGGCCGCGGGCGGCGACGGGGCCCGTGCGT |
| | | CCCAGCGCACATGTTCGGCGAGGCGGGGCCTGCGA |
| | | GCGCGGCCACCGAGAATCGGACGGGGGTAGTCTCA |
| | | AGCTGGCCGGCCTGCTCTGGTGCCTGGCCTCGCGC |
| | | CGCCGTGTATCGCCCCGCCCTGGGCGGCAAGGCTG |
| | | GCCCGGTCGGCACCAGTTGCGTGAGCGGAAAGATG |
| | | GCCGCTTCCCGGCCCTGCTGCAGGGAGCTCAAAAT |
| | | GGAGGACGCGGCGCTCGGGAGAGCGGGCGGGTGAG |
| | | TCACCCACACAAAGGAAAAGGGCCTTTCCGTCCTC |
| | | AGCCGTCGCTTCATGTGACTCCACTGAGTACCGGG |
| | | CGCCGTCCAGGCACCTCGATTAGTTCTCGTGCTTT |
| | | TGGAGTACGTCGTCTTTAGGTTGGGGGGAGGGGTT |
| | | TTATGCGATGGAGTTTCCCCACACTGAGTGGGTGG |
| | | AGACTGAAGTTAGGCCAGCTTGGCACTTGATGTAA |
| | | TTCTCCTTGGAATTTGCCCTTTTTGAGTTTGGATC |
| | | TTGGTTCATTCTCAAGCCTCAGACAGTGGTTCAAA |
| | | GTTTTTTTCTTCCATTTCAGGTGTCGTGAAGCTCT |
| | | AGAGCCATGGCCCTCCCTGTCACCGCCCTGCTGCT |
| | | TCCGCTGGCTCTTCTGCTCCACGCCGCTCGGCCCG |
| | | AGGTGAAACTGCAGGAGTCAGGACCTGGCCTGGTG |
| | | GCGCCCTCACAGAGCCTGTCCGTCACATGCACTGT |
| | | CTCAGGGGTCTCATTACCCGACTATGGTGTAAGCT |
| | | GGATTCGCCAGCCTCCACGAAAGGGTCTGGAGTGG |
| | | CTGGGAGTAATATGGGGTAGTGAAACCACATACTA |
| | | TAATTCAGCTCTCAAATCCAGACTGACCATCATCA |
| | | AGGACAACTCCAAGAGCCAAGTTTTCTTAAAAATG |
| | | AACAGTCTGCAAACTGATGACACAGCCATTTACTA |
| | | CTGTGCCAAACATTATTACTACGGTGGTAGCTATG |
| | | CTATGGACTACTGGGGTCAAGGAACCTCAGTCACC |
| | | GTCTCCTCAGGTGGAGGTGGCAGCGGAGGAGGTGG |
| | | GTCCGGCGGTGGAGGAAGCGGCGGTGGAGGAAGCG |
| | | ACATCCAGATGACACAGACTACATCCTCCCTGTCT |
| | | GCCTCTCTGGGAGACAGAGTCACCATCAGTTGCAG |
| | | GGCAAGTCAGGACATTAGTAAATATTTAAATTGGT |
| | | ATCAGCAGAAACCAGATGGAACTGTTAAACTCCTG |
| | | ATCTACCATACATCAAGATTACACTCAGGAGTCCC |
| | | ATCAAGGTTCAGTGGCAGTGGGTCTGGAACAGATT |
| | | ATTCTCTCACCATTAGCAACCTGGAGCAAGAAGAT |
| | | ATTGCCACTTACTTTTGCCAACAGGGTAATACGCT |
| | | TCCGTACACGTTCGGAGGGGGGACTAAGTTGGAAA |
| | | TAACACGGGCTGATGCTGCACCAACTGTATCCATC |
| | | TTCCCACCATCCAGTAATACCACTACCCCAGCACC |
| | | GAGGCCACCCACCCCGGCTCCTACCATCGCCTCCC |
| | | AGCCTCTGTCCCTGCGTCCGGAGGCATGTAGACCC |
| | | GCAGCTGGTGGGGCCGTGCATACCCGGGGTCTTGA |
| | | CTTCGCCTGCGATATCTACATTTGGGCCCCTCTGG |
| | | CTGGTACTTGCGGGGTCCTGCTGCTTTCACTCGTG |
| | | ATCACTCTTTACTGTAAGCGCGGTCGGAAGAAGCT |
| | | GCTGTACATCTTTAAGCAACCCTTCATGAGGCCTG |
| | | TGCAGACTACTCAAGAGGAGGACGGCTGTTCATGC |
| | | CGGTTCCCAGAGGAGGAGGAAGGCGGCTGCGAACT |
| | | GCGCGTGAAATTCAGCCGCAGCGCAGATGCTCCAG |
| | | CCTACCAACAGGGGCAGAACCAGCTCTACAACGAA |
| | | CTCAATCTTGGTCGGAGAGAGGAGTACGACGTGCT |
| | | GGACAAGCGGAGAGGACGGGACCCAGAAATGGGCG |
| | | GGAAGCCGCGCAGAAAGAATCCCCAAGAGGGCCTG |
| | | TACAACGAGCTCCAAAAGGATAAGATGGCAGAAGC |
| | | CTATAGCGAGATTGGTATGAAAGGGGAACGCAGAA |
| | | GAGGCAAAGGCCACGACGGACTGTACCAGGGACTC |
| | | AGCACCGCCACCAAGGACACCTATGACGCTCTTCA |
| | | CATGCAGGCCCTGCCGCCTCGGTAAGTCGACAATC |
| | | AACCTCTGGATTACAAAATTTGTGAAAGATTGACT |
| | | GGTATTCTTAACTATGTTGCTCCTTTTACGCTATG |
| | | TGGATACGCTGCTTTAATGCCTTTGTATCATGCTA |
| | | TTGCTTCCCGTATGGCTTTCATTTTCTCCTCCTTG |
| | | TATAAATCCTGGTTGCTGTCTCTTTATGAGGAGTT |
| | | GTGGCCCGTTGTCAGGCAACGTGGCGTGGTGTGCA |
| | | CTGTGTTTGCTGACGCAACCCCCACTGGTTGGGGC |
| | | ATTGCCACCACCTGTCAGCTCCTTTCCGGGACTTT |
| | | CGCTTTCCCCCTCCCTATTGCCACGGCGGAACTCA |
| | | TCGCCGCCTGCCTTGCCCGCTGCTGGACAGGGGCT |
| | | CGGCTGTTGGGCACTGACAATTCCGTGGTGTTGTC |
| | | GGGGAAGCTGACGTCCTTTCCTTGGCTGCTCGCCT |
| | | GTGTTGCCACCTGGATTCTGCGCGGGACGTCCTTC |
| | | TGCTACGTCCCTTCGGCCCTCAATCCAGCGGACCT |
| | | TCCTTCCCGCGGCCTGCTGCCGGCTCTGCGGCCTC |
| | | TTCCGCGTCTTCGCCTTCGCCCTCAGACGAGTCGG |
| | | ATCTCCCTTTGGGCCGCCTCCCCGC |
| 19 | Stealth 1 construct | GAGGGCCTATTTCCCATGATTCCTTCATATTTGCA |
| | (CIITA shRNA) | TATACGATACAAGGCTGTTAGAGAGATAATTAGAA |
| | hU6 + shRNA | TTAATTTGACTGTAAACACAAAGATATTAGTACAA |
| | targeting CIITA | AATACGTGACGTAGAAAGTAATAATTTCTTGGGTA |
| | | GTTTGCAGTTTTAAAATTATGTTTTAAAATGGACT |
| | | ATCATATGCTTACCGTAACTTGAAAGTATTTCGAT |
| | | TTCTTGGCTTTATATATCTTGTGGAAAGGACGAAA |
| | | CACCGAACAACAGGATTCACGGATCAGCTTCAAGA |
| | | GAGCTGATCCGTGAATCCTGTTGTTTTTTTT |
| 20 | Stealth 1 construct | CGTGAGGCTCCGGTGCCCGTCAGTGGGCAGAGCGC |
| | (CAR − TAPi) | ACATCGCCCACAGTCCCCGAGAAGTTGGGGGGAGG |
| | EF1a + aCD19 CAR | GGTCGGCAATTGAACCGGTGCCTAGAGAAGGTGGC |
| | + T2A + EBV | GCGGGGTAAACTGGGAAAGTGATGTCGTGTACTGG |
| | BNLF2a + P2A + | CTCCGCCTTTTTCCCGAGGGTGGGGGAGAACCGTA |
| | eGFP + WHP | TATAAGTGCAGTAGTCGCCGTGAACGTTCTTTTTC |
| | | GCAACGGGTTTGCCGCCAGAACACAGGTAAGTGCC |
| | | GTGTGTGGTTCCCGCGGGCCTGGCCTCTTTACGGG |
| | | TTATGGCCCTTGCGTGCCTTGAATTACTTCCACCT |
| | | GGCTGCAGTACGTGATTCTTGATCCCGAGCTTCGG |
| | | GTTGGAAGTGGGTGGGAGAGTTCGAGGCCTTGCGC |
| | | TTAAGGAGCCCCTTCGCCTCGTGCTTGAGTTGAGG |
| | | CCTGGCCTGGGCGCTGGGGCCGCCGCGTGCGAATC |
| | | TGGTGGCACCTTCGCGCCTGTCTCGCTGCTTTCGA |
| | | TAAGTCTCTAGCCATTTAAAATTTTTGATGACCTG |
| | | CTGCGACGCTTTTTTTCTGGCAAGATAGTCTTGTA |
| | | AATGCGGGCCAAGATCTGCACACTGGTATTTCGGT |
| | | TTTTGGGGCCGCGGGCGGCGACGGGGCCCGTGCGT |
| | | CCCAGCGCACATGTTCGGCGAGGCGGGGCCTGCGA |
| | | GCGCGGCCACCGAGAATCGGACGGGGGTAGTCTCA |
| | | AGCTGGCCGGCCTGCTCTGGTGCCTGGCCTCGCGC |
| | | CGCCGTGTATCGCCCCGCCCTGGGCGGCAAGGCTG |
| | | GCCCGGTCGGCACCAGTTGCGTGAGCGGAAAGATG |
| | | GCCGCTTCCCGGCCCTGCTGCAGGGAGCTCAAAAT |
| | | GGAGGACGCGGCGCTCGGGAGAGCGGGCGGGTGAG |
| | | TCACCCACACAAAGGAAAAGGGCCTTTCCGTCCTC |
| | | AGCCGTCGCTTCATGTGACTCCACTGAGTACCGGG |
| | | CGCCGTCCAGGCACCTCGATTAGTTCTCGTGCTTT |
| | | TGGAGTACGTCGTCTTTAGGTTGGGGGGAGGGGTT |
| | | TTATGCGATGGAGTTTCCCCACACTGAGTGGGTGG |
| | | AGACTGAAGTTAGGCCAGCTTGGCACTTGATGTAA |
| | | TTCTCCTTGGAATTTGCCCTTTTTGAGTTTGGATC |
| | | TTGGTTCATTCTCAAGCCTCAGACAGTGGTTCAAA |
| | | GTTTTTTTCTTCCATTTCAGGTGTCGTGAAGCTCT |
| | | AGAGCCATGGCCCTCCCTGTCACCGCCCTGCTGCT |
| | | TCCGCTGGCTCTTCTGCTCCACGCCGCTCGGCCCG |
| | | AGGTGAAACTGCAGGAGTCAGGACCTGGCCTGGTG |
| | | GCGCCCTCACAGAGCCTGTCCGTCACATGCACTGT |
| | | CTCAGGGGTCTCATTACCCGACTATGGTGTAAGCT |
| | | GGATTCGCCAGCCTCCACGAAAGGGTCTGGAGTGG |
| | | CTGGGAGTAATATGGGGTAGTGAAACCACATACTA |
| | | TAATTCAGCTCTCAAATCCAGACTGACCATCATCA |
| | | AGGACAACTCCAAGAGCCAAGTTTTCTTAAAAATG |
| | | AACAGTCTGCAAACTGATGACACAGCCATTTACTA |
| | | CTGTGCCAAACATTATTACTACGGTGGTAGCTATG |
| | | CTATGGACTACTGGGGTCAAGGAACCTCAGTCACC |
| | | GTCTCCTCAGGTGGAGGTGGCAGCGGAGGAGGTGG |
| | | GTCCGGCGGTGGAGGAAGCGGCGGTGGAGGAAGCG |
| | | ACATCCAGATGACACAGACTACATCCTCCCTGTCT |
| | | GCCTCTCTGGGAGACAGAGTCACCATCAGTTGCAG |
| | | GGCAAGTCAGGACATTAGTAAATATTTAAATTGGT |
| | | ATCAGCAGAAACCAGATGGAACTGTTAAACTCCTG |
| | | ATCTACCATACATCAAGATTACACTCAGGAGTCCC |
| | | ATCAAGGTTCAGTGGCAGTGGGTCTGGAACAGATT |
| | | ATTCTCTCACCATTAGCAACCTGGAGCAAGAAGAT |
| | | ATTGCCACTTACTTTTGCCAACAGGGTAATACGCT |
| | | TCCGTACACGTTCGGAGGGGGGACTAAGTTGGAAA |
| | | TAACACGGGCTGATGCTGCACCAACTGTATCCATC |
| | | TTCCCACCATCCAGTAATACCACTACCCCAGCACC |
| | | GAGGCCACCCACCCCGGCTCCTACCATCGCCTCCC |
| | | AGCCTCTGTCCCTGCGTCCGGAGGCATGTAGACCC |
| | | GCAGCTGGTGGGGCCGTGCATACCCGGGGTCTTGA |
| | | CTTCGCCTGCGATATCTACATTTGGGCCCCTCTGG |
| | | CTGGTACTTGCGGGGTCCTGCTGCTTTCACTCGTG |
| | | ATCACTCTTTACTGTAAGCGCGGTCGGAAGAAGCT |
| | | GCTGTACATCTTTAAGCAACCCTTCATGAGGCCTG |
| | | TGCAGACTACTCAAGAGGAGGACGGCTGTTCATGC |
| | | CGGTTCCCAGAGGAGGAGGAAGGCGGCTGCGAACT |
| | | GCGCGTGAAATTCAGCCGCAGCGCAGATGCTCCAG |
| | | CCTACCAACAGGGGCAGAACCAGCTCTACAACGAA |
| | | CTCAATCTTGGTCGGAGAGAGGAGTACGACGTGCT |
| | | GGACAAGCGGAGAGGACGGGACCCAGAAATGGGCG |
| | | GGAAGCCGCGCAGAAAGAATCCCCAAGAGGGCCTG |
| | | TACAACGAGCTCCAAAAGGATAAGATGGCAGAAGC |
| | | CTATAGCGAGATTGGTATGAAAGGGGAACGCAGAA |
| | | GAGGCAAAGGCCACGACGGACTGTACCAGGGACTC |
| | | AGCACCGCCACCAAGGACACCTATGACGCTCTTCA |
| | | CATGCAGGCCCTGCCGCCTCGGGGCTCAGGAGAGG |
| | | GCAGAGGAAGTCTTCTAACATGCGGTGACGTGGAG |
| | | GAGAATCCCGGCCCTATGGTACACGTATTGGAACG |
| | | GGCGCTTTTGGAGCAGCAAAGCTCCGCGTGCGGAC |
| | | TCCCAGGTTCATCCACGGAAACACGCCCATCTCAT |
| | | CCCTGCCCGGAGGACCCCGATGTATCACGACTTAG |
| | | GCTCTTGCTGGTCGTACTTTGCGTGCTCTTTGGAC |
| | | TTCTGTGCCTCCTGCTCATCGGCTCAGGAGGTTCA |
| | | GGTGCAACGAACTTCTCATTGTTGAAGCAAGCCGG |
| | | TGATGTTGAGGAAAATCCGGGTCCTATGGTGAGCA |
| | | AGGGCGAGGAGCTGTTCACCGGGGTGGTGCCCATC |
| | | CTGGTCGAGCTGGACGGCGACGTAAACGGCCACAA |
| | | GTTCAGCGTGTCCGGCGAGGGCGAGGGCGATGCCA |
| | | CCTACGGCAAGCTGACCCTGAAGTTCATCTGCACC |
| | | ACCGGCAAGCTGCCCGTGCCCTGGCCCACCCTCGT |
| | | GACCACCCTGACCTACGGCGTGCAGTGCTTCAGCC |
| | | GCTACCCCGACCACATGAAGCAGCACGACTTCTTC |
| | | AAGTCCGCCATGCCCGAAGGCTACGTCCAGGAGCG |
| | | CACCATCTTCTTCAAGGACGACGGCAACTACAAGA |
| | | CCCGCGCCGAGGTGAAGTTCGAGGGCGACACCCTG |
| | | GTGAACCGCATCGAGCTGAAGGGCATCGACTTCAA |
| | | GGAGGACGGCAACATCCTGGGGCACAAGCTGGAGT |
| | | ACAACTACAACAGCCACAACGTCTATATCATGGCC |
| | | GACAAGCAGAAGAACGGCATCAAGGTGAACTTCAA |
| | | GATCCGCCACAACATCGAGGACGGCAGCGTGCAGC |
| | | TCGCCGACCACTACCAGCAGAACACCCCCATCGGC |
| | | GACGGCCCCGTGCTGCTGCCCGACAACCACTACCT |
| | | GAGCACCCAGTCCGCCCTGAGCAAAGACCCCAACG |
| | | AGAAGCGCGATCACATGGTCCTGCTGGAGTTCGTG |
| | | ACCGCCGCCGGGATCACTCTCGGCATGGACGAGCT |
| | | GTACAAGTAAGTCGACAATCAACCTCTGGATTACA |
| | | AAATTTGTGAAAGATTGACTGGTATTCTTAACTAT |
| | | GTTGCTCCTTTTACGCTATGTGGATACGCTGCTTT |
| | | AATGCCTTTGTATCATGCTATTGCTTCCCGTATGG |
| | | CTTTCATTTTCTCCTCCTTGTATAAATCCTGGTTG |
| | | CTGTCTCTTTATGAGGAGTTGTGGCCCGTTGTCAG |
| | | GCAACGTGGCGTGGTGTGCACTGTGTTTGCTGACG |
| | | CAACCCCCACTGGTTGGGGCATTGCCACCACCTGT |
| | | CAGCTCCTTTCCGGGACTTTCGCTTTCCCCCTCCC |
| | | TATTGCCACGGCGGAACTCATCGCCGCCTGCCTTG |
| | | CCCGCTGCTGGACAGGGGCTCGGCTGTTGGGCACT |
| | | GACAATTCCGTGGTGTTGTCGGGGAAGCTGACGTC |
| | | CTTTCCTTGGCTGCTCGCCTGTGTTGCCACCTGGA |
| | | TTCTGCGCGGGACGTCCTTCTGCTACGTCCCTTCG |
| | | GCCCTCAATCCAGCGGACCTTCCTTCCCGCGGCCT |
| | | GCTGCCGGCTCTGCGGCCTCTTCCGCGTCTTCGCC |
| | | TTCGCCCTCAGACGAGTCGGATCTCCCTTTGGGCC |
| | | GCCTCCCCGCCTG |
| 21 | Stealth 2 construct | GAGGGCCTATTTCCCATGATTCCTTCATATTTGCA |
| | (CIITA shRNA) | TATACGATACAAGGCTGTTAGAGAGATAATTAGAA |
| | hU6 + shRNA | TTAATTTGACTGTAAACACAAAGATATTAGTACAA |
| | targeting CIITA | AATACGTGACGTAGAAAGTAATAATTTCTTGGGTA |
| | | GTTTGCAGTTTTAAAATTATGTTTTAAAATGGACT |
| | | ATCATATGCTTACCGTAACTTGAAAGTATTTCGAT |
| | | TTCTTGGCTTTATATATCTTGTGGAAAGGACGAAA |
| | | CACCGAACAACAGGATTCACGGATCAGCTTCAAGA |
| | | GAGCTGATCCGTGAATCCTGTTGTTTTTTTT |
| 22 | Stealth 2 construct | GGCTCCGGTGCCCGTCAGTGGGCAGAGCGCACATC |
| | (CAR − TAPi) | GCCCACAGTCCCCGAGAAGTTGGGGGGAGGGGTCG |
| | EF1a + aCD19 CAR | GCAATTGAACCGGTGCCTAGAGAAGGTGGCGCGGG |
| | + T2A + eGFP + | GTAAACTGGGAAAGTGATGTCGTGTACTGGCTCCG |
| | P2A + EBV BNLF2a | CCTTTTTCCCGAGGGTGGGGGAGAACCGTATATAA |
| | + WHP | GTGCAGTAGTCGCCGTGAACGTTCTTTTTCGCAAC |
| | | GGGTTTGCCGCCAGAACACAGGTAAGTGCCGTGTG |
| | | TGGTTCCCGCGGGCCTGGCCTCTTTACGGGTTATG |
| | | GCCCTTGCGTGCCTTGAATTACTTCCACCTGGCTG |
| | | CAGTACGTGATTCTTGATCCCGAGCTTCGGGTTGG |
| | | AAGTGGGTGGGAGAGTTCGAGGCCTTGCGCTTAAG |
| | | GAGCCCCTTCGCCTCGTGCTTGAGTTGAGGCCTGG |
| | | CCTGGGCGCTGGGGCCGCCGCGTGCGAATCTGGTG |
| | | GCACCTTCGCGCCTGTCTCGCTGCTTTCGATAAGT |
| | | CTCTAGCCATTTAAAATTTTTGATGACCTGCTGCG |
| | | ACGCTTTTTTTCTGGCAAGATAGTCTTGTAAATGC |
| | | GGGCCAAGATCTGCACACTGGTATTTCGGTTTTTG |
| | | GGGCCGCGGGCGGCGACGGGGCCCGTGCGTCCCAG |
| | | CGCACATGTTCGGCGAGGCGGGGCCTGCGAGCGCG |
| | | GCCACCGAGAATCGGACGGGGGTAGTCTCAAGCTG |
| | | GCCGGCCTGCTCTGGTGCCTGGCCTCGCGCCGCCG |
| | | TGTATCGCCCCGCCCTGGGCGGCAAGGCTGGCCCG |
| | | GTCGGCACCAGTTGCGTGAGCGGAAAGATGGCCGC |
| | | TTCCCGGCCCTGCTGCAGGGAGCTCAAAATGGAGG |
| | | ACGCGGCGCTCGGGAGAGCGGGCGGGTGAGTCACC |
| | | CACACAAAGGAAAAGGGCCTTTCCGTCCTCAGCCG |
| | | TCGCTTCATGTGACTCCACTGAGTACCGGGCGCCG |
| | | TCCAGGCACCTCGATTAGTTCTCGTGCTTTTGGAG |
| | | TACGTCGTCTTTAGGTTGGGGGGAGGGGTTTTATG |
| | | CGATGGAGTTTCCCCACACTGAGTGGGTGGAGACT |
| | | GAAGTTAGGCCAGCTTGGCACTTGATGTAATTCTC |
| | | CTTGGAATTTGCCCTTTTTGAGTTTGGATCTTGGT |
| | | TCATTCTCAAGCCTCAGACAGTGGTTCAAAGTTTT |
| | | TTTCTTCCATTTCAGGTGTCGTGAAGCTCTAGAGC |
| | | CATGGCCCTCCCTGTCACCGCCCTGCTGCTTCCGC |
| | | TGGCTCTTCTGCTCCACGCCGCTCGGCCCGAGGTG |
| | | AAACTGCAGGAGTCAGGACCTGGCCTGGTGGCGCC |
| | | CTCACAGAGCCTGTCCGTCACATGCACTGTCTCAG |
| | | GGGTCTCATTACCCGACTATGGTGTAAGCTGGATT |
| | | CGCCAGCCTCCACGAAAGGGTCTGGAGTGGCTGGG |
| | | AGTAATATGGGGTAGTGAAACCACATACTATAATT |
| | | CAGCTCTCAAATCCAGACTGACCATCATCAAGGAC |
| | | AACTCCAAGAGCCAAGTTTTCTTAAAAATGAACAG |
| | | TCTGCAAACTGATGACACAGCCATTTACTACTGTG |
| | | CCAAACATTATTACTACGGTGGTAGCTATGCTATG |
| | | GACTACTGGGGTCAAGGAACCTCAGTCACCGTCTC |
| | | CTCAGGTGGAGGTGGCAGCGGAGGAGGTGGGTCCG |
| | | GCGGTGGAGGAAGCGGCGGTGGAGGAAGCGACATC |
| | | CAGATGACACAGACTACATCCTCCCTGTCTGCCTC |
| | | TCTGGGAGACAGAGTCACCATCAGTTGCAGGGCAA |
| | | GTCAGGACATTAGTAAATATTTAAATTGGTATCAG |
| | | CAGAAACCAGATGGAACTGTTAAACTCCTGATCTA |
| | | CCATACATCAAGATTACACTCAGGAGTCCCATCAA |
| | | GGTTCAGTGGCAGTGGGTCTGGAACAGATTATTCT |
| | | CTCACCATTAGCAACCTGGAGCAAGAAGATATTGC |
| | | CACTTACTTTTGCCAACAGGGTAATACGCTTCCGT |
| | | ACACGTTCGGAGGGGGGACTAAGTTGGAAATAACA |
| | | CGGGCTGATGCTGCACCAACTGTATCCATCTTCCC |
| | | ACCATCCAGTAATACCACTACCCCAGCACCGAGGC |
| | | CACCCACCCCGGCTCCTACCATCGCCTCCCAGCCT |
| | | CTGTCCCTGCGTCCGGAGGCATGTAGACCCGCAGC |
| | | TGGTGGGGCCGTGCATACCCGGGGTCTTGACTTCG |
| | | CCTGCGATATCTACATTTGGGCCCCTCTGGCTGGT |
| | | ACTTGCGGGGTCCTGCTGCTTTCACTCGTGATCAC |
| | | TCTTTACTGTAAGCGCGGTCGGAAGAAGCTGCTGT |
| | | ACATCTTTAAGCAACCCTTCATGAGGCCTGTGCAG |
| | | ACTACTCAAGAGGAGGACGGCTGTTCATGCCGGTT |
| | | CCCAGAGGAGGAGGAAGGCGGCTGCGAACTGCGCG |
| | | TGAAATTCAGCCGCAGCGCAGATGCTCCAGCCTAC |
| | | CAACAGGGGCAGAACCAGCTCTACAACGAACTCAA |
| | | TCTTGGTCGGAGAGAGGAGTACGACGTGCTGGACA |
| | | AGCGGAGAGGACGGGACCCAGAAATGGGCGGGAAG |
| | | CCGCGCAGAAAGAATCCCCAAGAGGGCCTGTACAA |
| | | CGAGCTCCAAAAGGATAAGATGGCAGAAGCCTATA |
| | | GCGAGATTGGTATGAAAGGGGAACGCAGAAGAGGC |
| | | AAAGGCCACGACGGACTGTACCAGGGACTCAGCAC |
| | | CGCCACCAAGGACACCTATGACGCTCTTCACATGC |
| | | AGGCCCTGCCGCCTCGGGGCTCAGGAGAGGGCAGA |
| | | GGAAGTCTTCTAACATGCGGTGACGTGGAGGAGAA |
| | | TCCCGGCCCTATGGTGAGCAAGGGCGAGGAGCTGT |
| | | TCACCGGGGTGGTGCCCATCCTGGTCGAGCTGGAC |
| | | GGCGACGTAAACGGCCACAAGTTCAGCGTGTCCGG |
| | | CGAGGGCGAGGGCGATGCCACCTACGGCAAGCTGA |
| | | CCCTGAAGTTCATCTGCACCACCGGCAAGCTGCCC |
| | | GTGCCCTGGCCCACCCTCGTGACCACCCTGACCTA |
| | | CGGCGTGCAGTGCTTCAGCCGCTACCCCGACCACA |
| | | TGAAGCAGCACGACTTCTTCAAGTCCGCCATGCCC |
| | | GAAGGCTACGTCCAGGAGCGCACCATCTTCTTCAA |
| | | GGACGACGGCAACTACAAGACCCGCGCCGAGGTGA |
| | | AGTTCGAGGGCGACACCCTGGTGAACCGCATCGAG |
| | | CTGAAGGGCATCGACTTCAAGGAGGACGGCAACAT |
| | | CCTGGGGCACAAGCTGGAGTACAACTACAACAGCC |
| | | ACAACGTCTATATCATGGCCGACAAGCAGAAGAAC |
| | | GGCATCAAGGTGAACTTCAAGATCCGCCACAACAT |
| | | CGAGGACGGCAGCGTGCAGCTCGCCGACCACTACC |
| | | AGCAGAACACCCCCATCGGCGACGGCCCCGTGCTG |
| | | CTGCCCGACAACCACTACCTGAGCACCCAGTCCGC |
| | | CCTGAGCAAAGACCCCAACGAGAAGCGCGATCACA |
| | | TGGTCCTGCTGGAGTTCGTGACCGCCGCCGGGATC |
| | | ACTCTCGGCATGGACGAGCTGTACAAGGGTTCAGG |
| | | TGCAACGAACTTCTCATTGTTGAAGCAAGCCGGTG |
| | | ATGTTGAGGAAAATCCGGGTCCTATGGTACACGTA |
| | | TTGGAACGGGCGCTTTTGGAGCAGCAAAGCTCCGC |
| | | GTGCGGACTCCCAGGTTCATCCACGGAAACACGCC |
| | | CATCTCATCCCTGCCCGGAGGACCCCGATGTATCA |
| | | CGACTTAGGCTCTTGCTGGTCGTACTTTGCGTGCT |
| | | CTTTGGACTTCTGTGCCTCCTGCTCATCGGCTCAG |
| | | GATAAGTCGACAATCAACCTCTGGATTACAAAATT |
| | | TGTGAAAGATTGACTGGTATTCTTAACTATGTTGC |
| | | TCCTTTTACGCTATGTGGATACGCTGCTTTAATGC |
| | | CTTTGTATCATGCTATTGCTTCCCGTATGGCTTTC |
| | | ATTTTCTCCTCCTTGTATAAATCCTGGTTGCTGTC |
| | | TCTTTATGAGGAGTTGTGGCCCGTTGTCAGGCAAC |
| | | GTGGCGTGGTGTGCACTGTGTTTGCTGACGCAACC |
| | | CCCACTGGTTGGGGCATTGCCACCACCTGTCAGCT |
| | | CCTTTCCGGGACTTTCGCTTTCCCCCTCCCTATTG |
| | | CCACGGCGGAACTCATCGCCGCCTGCCTTGCCCGC |
| | | TGCTGGACAGGGGCTCGGCTGTTGGGCACTGACAA |
| | | TTCCGTGGTGTTGTCGGGGAAGCTGACGTCCTTTC |
| | | CTTGGCTGCTCGCCTGTGTTGCCACCTGGATTCTG |
| | | CGCGGGACGTCCTTCTGCTACGTCCCTTCGGCCCT |
| | | CAATCCAGCGGACCTTCCTTCCCGCGGCCTGCTGC |
| | | CGGCTCTGCGGCCTCTTCCGCGTCTTCGCCTTCGC |
| | | CCTCAGACGAGTCGGATCTCCCTTTGGGCCGCCTC |
| | | CCCGCCTG |
| 23 | Stealth construct | GAGGGCCTATTTCCCATGATTCCTTCATATTTGCA |
| | no GFP | TATACGATACAAGGCTGTTAGAGAGATAATTAGAA |
| | (CIITA shRNA) | TTAATTTGACTGTAAACACAAAGATATTAGTACAA |
| | hU6 + shRNA | AATACGTGACGTAGAAAGTAATAATTTCTTGGGTA |
| | targeting CIITA | GTTTGCAGTTTTAAAATTATGTTTTAAAATGGACT |
| | | ATCATATGCTTACCGTAACTTGAAAGTATTTCGAT |
| | | TTCTTGGCTTTATATATCTTGTGGAAAGGACGAAA |
| | | CACCGAACAACAGGATTCACGGATCAGCTTCAAGA |
| | | GAGCTGATCCGTGAATCCTGTTGTTTTTTTT |
| 24 | Stealth construct | GGCTCCGGTGCCCGTCAGTGGGCAGAGCGCACATC |
| | no GFP | GCCCACAGTCCCCGAGAAGTTGGGGGGAGGGGTCG |
| | (CAR − TAPi) | GCAATTGAACCGGTGCCTAGAGAAGGTGGCGCGGG |
| | EF1a + aCD19 CAR | GTAAACTGGGAAAGTGATGTCGTGTACTGGCTCCG |
| | + T2A + EBV | CCTTTTTCCCGAGGGTGGGGGAGAACCGTATATAA |
| | BNLF2a + WHP | GTGCAGTAGTCGCCGTGAACGTTCTTTTTCGCAAC |
| | | GGGTTTGCCGCCAGAACACAGGTAAGTGCCGTGTG |
| | | TGGTTCCCGCGGGCCTGGCCTCTTTACGGGTTATG |
| | | GCCCTTGCGTGCCTTGAATTACTTCCACCTGGCTG |
| | | CAGTACGTGATTCTTGATCCCGAGCTTCGGGTTGG |
| | | AAGTGGGTGGGAGAGTTCGAGGCCTTGCGCTTAAG |
| | | GAGCCCCTTCGCCTCGTGCTTGAGTTGAGGCCTGG |
| | | CCTGGGCGCTGGGGCCGCCGCGTGCGAATCTGGTG |
| | | GCACCTTCGCGCCTGTCTCGCTGCTTTCGATAAGT |
| | | CTCTAGCCATTTAAAATTTTTGATGACCTGCTGCG |
| | | ACGCTTTTTTTCTGGCAAGATAGTCTTGTAAATGC |
| | | GGGCCAAGATCTGCACACTGGTATTTCGGTTTTTG |
| | | GGGCCGCGGGCGGCGACGGGGCCCGTGCGTCCCAG |
| | | CGCACATGTTCGGCGAGGCGGGGCCTGCGAGCGCG |
| | | GCCACCGAGAATCGGACGGGGGTAGTCTCAAGCTG |
| | | GCCGGCCTGCTCTGGTGCCTGGCCTCGCGCCGCCG |
| | | TGTATCGCCCCGCCCTGGGCGGCAAGGCTGGCCCG |
| | | GTCGGCACCAGTTGCGTGAGCGGAAAGATGGCCGC |
| | | TTCCCGGCCCTGCTGCAGGGAGCTCAAAATGGAGG |
| | | ACGCGGCGCTCGGGAGAGCGGGCGGGTGAGTCACC |
| | | CACACAAAGGAAAAGGGCCTTTCCGTCCTCAGCCG |
| | | TCGCTTCATGTGACTCCACTGAGTACCGGGCGCCG |
| | | TCCAGGCACCTCGATTAGTTCTCGTGCTTTTGGAG |
| | | TACGTCGTCTTTAGGTTGGGGGGAGGGGTTTTATG |
| | | CGATGGAGTTTCCCCACACTGAGTGGGTGGAGACT |
| | | GAAGTTAGGCCAGCTTGGCACTTGATGTAATTCTC |
| | | CTTGGAATTTGCCCTTTTTGAGTTTGGATCTTGGT |
| | | TCATTCTCAAGCCTCAGACAGTGGTTCAAAGTTTT |
| | | TTTCTTCCATTTCAGGTGTCGTGAAGCTCTAGAGC |
| | | CATGGCCCTCCCTGTCACCGCCCTGCTGCTTCCGC |
| | | TGGCTCTTCTGCTCCACGCCGCTCGGCCCGAGGTG |
| | | AAACTGCAGGAGTCAGGACCTGGCCTGGTGGCGCC |
| | | CTCACAGAGCCTGTCCGTCACATGCACTGTCTCAG |
| | | GGGTCTCATTACCCGACTATGGTGTAAGCTGGATT |
| | | CGCCAGCCTCCACGAAAGGGTCTGGAGTGGCTGGG |
| | | AGTAATATGGGGTAGTGAAACCACATACTATAATT |
| | | CAGCTCTCAAATCCAGACTGACCATCATCAAGGAC |
| | | AACTCCAAGAGCCAAGTTTTCTTAAAAATGAACAG |
| | | TCTGCAAACTGATGACACAGCCATTTACTACTGTG |
| | | CCAAACATTATTACTACGGTGGTAGCTATGCTATG |
| | | GACTACTGGGGTCAAGGAACCTCAGTCACCGTCTC |
| | | CTCAGGTGGAGGTGGCAGCGGAGGAGGTGGGTCCG |
| | | GCGGTGGAGGAAGCGGCGGTGGAGGAAGCGACATC |
| | | CAGATGACACAGACTACATCCTCCCTGTCTGCCTC |
| | | TCTGGGAGACAGAGTCACCATCAGTTGCAGGGCAA |
| | | GTCAGGACATTAGTAAATATTTAAATTGGTATCAG |
| | | CAGAAACCAGATGGAACTGTTAAACTCCTGATCTA |
| | | CCATACATCAAGATTACACTCAGGAGTCCCATCAA |
| | | GGTTCAGTGGCAGTGGGTCTGGAACAGATTATTCT |
| | | CTCACCATTAGCAACCTGGAGCAAGAAGATATTGC |
| | | CACTTACTTTTGCCAACAGGGTAATACGCTTCCGT |
| | | ACACGTTCGGAGGGGGGACTAAGTTGGAAATAACA |
| | | CGGGCTGATGCTGCACCAACTGTATCCATCTTCCC |
| | | ACCATCCAGTAATACCACTACCCCAGCACCGAGGC |
| | | CACCCACCCCGGCTCCTACCATCGCCTCCCAGCCT |
| | | CTGTCCCTGCGTCCGGAGGCATGTAGACCCGCAGC |
| | | TGGTGGGGCCGTGCATACCCGGGGTCTTGACTTCG |
| | | CCTGCGATATCTACATTTGGGCCCCTCTGGCTGGT |
| | | ACTTGCGGGGTCCTGCTGCTTTCACTCGTGATCAC |
| | | TCTTTACTGTAAGCGCGGTCGGAAGAAGCTGCTGT |
| | | ACATCTTTAAGCAACCCTTCATGAGGCCTGTGCAG |
| | | ACTACTCAAGAGGAGGACGGCTGTTCATGCCGGTT |
| | | CCCAGAGGAGGAGGAAGGCGGCTGCGAACTGCGCG |
| | | TGAAATTCAGCCGCAGCGCAGATGCTCCAGCCTAC |
| | | CAACAGGGGCAGAACCAGCTCTACAACGAACTCAA |
| | | TCTTGGTCGGAGAGAGGAGTACGACGTGCTGGACA |
| | | AGCGGAGAGGACGGGACCCAGAAATGGGCGGGAAG |
| | | CCGCGCAGAAAGAATCCCCAAGAGGGCCTGTACAA |
| | | CGAGCTCCAAAAGGATAAGATGGCAGAAGCCTATA |
| | | GCGAGATTGGTATGAAAGGGGAACGCAGAAGAGGC |
| | | AAAGGCCACGACGGACTGTACCAGGGACTCAGCAC |
| | | CGCCACCAAGGACACCTATGACGCTCTTCACATGC |
| | | AGGCCCTGCCGCCTCGGGGCTCAGGAGAGGGCAGA |
| | | GGAAGTCTTCTAACATGCGGTGACGTGGAGGAGAA |
| | | TCCCGGCCCTATGGTACACGTATTGGAACGGGCGC |
| | | TTTTGGAGCAGCAAAGCTCCGCGTGCGGACTCCCA |
| | | GGTTCATCCACGGAAACACGCCCATCTCATCCCTG |
| | | CCCGGAGGACCCCGATGTATCACGACTTAGGCTCT |
| | | TGCTGGTCGTACTTTGCGTGCTCTTTGGACTTCTG |
| | | TGCCTCCTGCTCATCTAAATCAACCTCTGGATTAC |
| | | AAAATTTGTGAAAGATTGACTGGTATTCTTAACTA |
| | | TGTTGCTCCTTTTACGCTATGTGGATACGCTGCTT |
| | | TAATGCCTTTGTATCATGCTATTGCTTCCCGTATG |
| | | GCTTTCATTTTCTCCTCCTTGTATAAATCCTGGTT |
| | | GCTGTCTCTTTATGAGGAGTTGTGGCCCGTTGTCA |
| | | GGCAACGTGGCGTGGTGTGCACTGTGTTTGCTGAC |
| | | GCAACCCCCACTGGTTGGGGCATTGCCACCACCTG |
| | | TCAGCTCCTTTCCGGGACTTTCGCTTTCCCCCTCC |
| | | CTATTGCCACGGCGGAACTCATCGCCGCCTGCCTT |
| | | GCCCGCTGCTGGACAGGGGCTCGGCTGTTGGGCAC |
| | | TGACAATTCCGTGGTGTTGTCGGGGAAGCTGACGT |
| | | CCTTTCCTTGGCTGCTCGCCTGTGTTGCCACCTGG |
| | | ATTCTGCGCGGGACGTCCTTCTGCTACGTCCCTTC |
| | | GGCCCTCAATCCAGCGGACCTTCCTTCCCGCGGCC |
| | | TGCTGCCGGCTCTGCGGCCTCTTCCGCGTCTTCGC |
| | | CTTCGCCCTCAGACGAGTCGGATCTCCCTTTGGGC |
| | | CGCCTCCCCGCCTG |
| 25 | CD8 hinge/TM | TTTPAPRPPTPAPTIASQPLSLRPEACRPAAGGAV |
| | domain | HTRGLDFACDIYIWAPLAGTCGVLLLSLVITLYC |
| 26 | 4-1BB ICD | KRGRKKLLYIFKQPFMRPVQTTQEEDGCSCRFPEE |
| | | EEGGCEL |
| 27 | CD3-zeta ICD | RVKFSRSADAPAYQQGQNQLYNELNLGRREEYDVL |
| | | DKRRGRDPEMGGKPRRKNPQEGLYNELQKDKMAEA |
| | | YSEIGMKGERRRGKGHDGLYQGLSTATKDTYDALH |
| | | MQALPPR |
| 28 | CD8 signal peptide | MALPVTALLLPLALLLHAARP |
| 29 | IgK signal sequence | METDTLLLWVLLLWVPGSTGD |
| 30 | linker-0 (G4S) | GGGGS |
| 31 | linker-1 (G3S)3 | GGGSGGGSGGGS |
| 32 | linker-2 (G4S)3 | GGGGSGGGGSGGGGS |
| 33 | linker-3 (G4S)4 | GGGGSGGGGSGGGGSGGGGS |
| 34 | linker-4 | GSTSGSGKPGSGEGSTKG |
| 35 | linker-5 | GGSSRSSSSGGGGSGGGG |
| 36 | T2A | SGGGGEGRGSLLTCGDVEENPGPR |
| 37 | P2A | GSGATNFSLLKQAGDVEENPGP |
| 38 | Histidine tag | HHHHHH |
| 39 | CD19 scFv VH | EVKLQESGPGLVAPSQSLSVTCTVSGVSLPDYGVS |
| | | WIRQPPRKGLEWLGVIWGSETTYYNSALKSRLTII |
| | | KDNSKSQVFLKMNSLQTDDTAIYYCAKHYYYGGSY |
| | | AMDYWGQGTSVTVSS |
| 40 | CD19 scFv VL | DIQMTQTTSSLSASLGDRVTISCRASQDISKYLNW |
| | | YQQKPDGTVKLLIYHTSRLHSGVPSRFSGSGSGTD |
| | | YSLTISNLEQEDIATYFCQQGNTLPYTFGGGTKLE |
| | | ITRADAAPTVSIFPPSSN |
| 41 | CD19 CAR | MALPVTALLLPLALLLHAARPEVKLQESGPGLVAP |
| | | SQSLSVTCTVSGVSLPDYGVSWIRQPPRKGLEWLG |
| | | VIWGSETTYYNSALKSRLTIIKDNSKSQVFLKMNS |
| | | LQTDDTAIYYCAKHYYYGGSYAMDYWGQGTSVTVS |
| | | SGGGGSGGGGSGGGGSGGGGSDIQMTQTTSSLSAS |
| | | LGDRVTISCRASQDISKYLNWYQQKPDGTVKLLIY |
| | | HTSRLHSGVPSRFSGSGSGTDYSLTISNLEQEDIA |
| | | TYFCQQGNTLPYTFGGGTKLEITRADAAPTVSIFP |
| | | PSSNTTTPAPRPPTPAPTIASQPLSLRPEACRPAA |
| | | GGAVHTRGLDFACDIYIWAPLAGTCGVLLLSLVIT |
| | | LYCKRGRKKLLYIFKQPFMRPVQTTQEEDGCSCRF |
| | | PEEEEGGCELRVKFSRSADAPAYQQGQNQLYNELN |
| | | LGRREEYDVLDKRRGRDPEMGGKPRRKNPQEGLYN |
| | | ELQKDKMAEAYSEIGMKGERRRGKGHDGLYQGLST |
| | | ATKDTYDALHMQALPPR |

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior technology or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples, which in no way should be construed as being further limiting.

### EXAMPLES

### Example 1. Expression of Viral TAP Inhibitors in Primary T Cells Results in Decreased Cell Surface Levels of MHC Class I

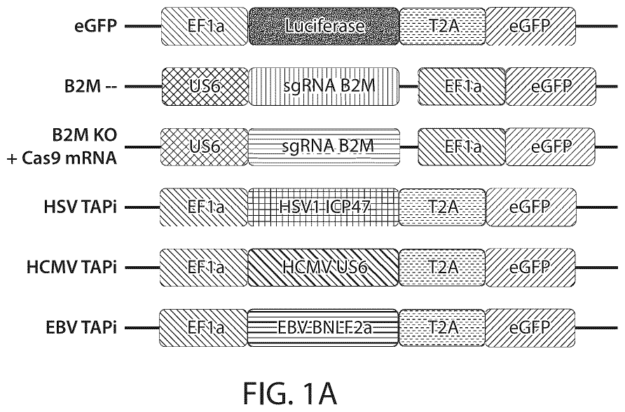

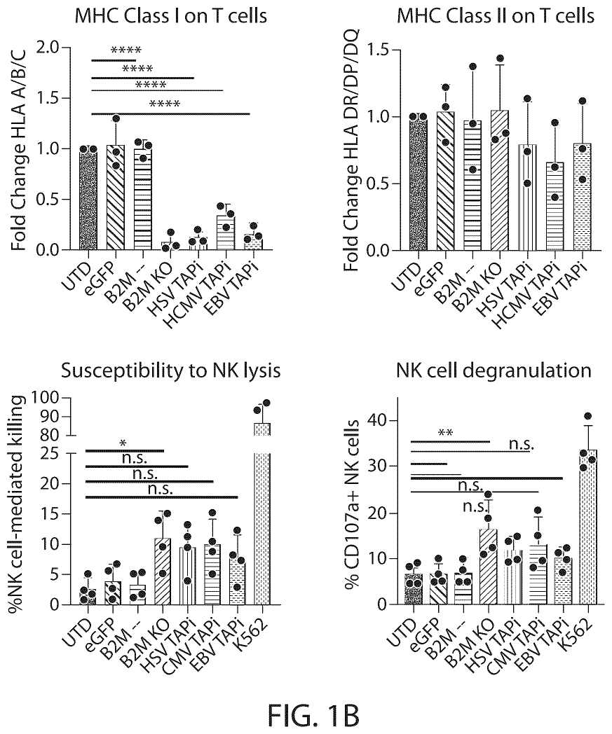

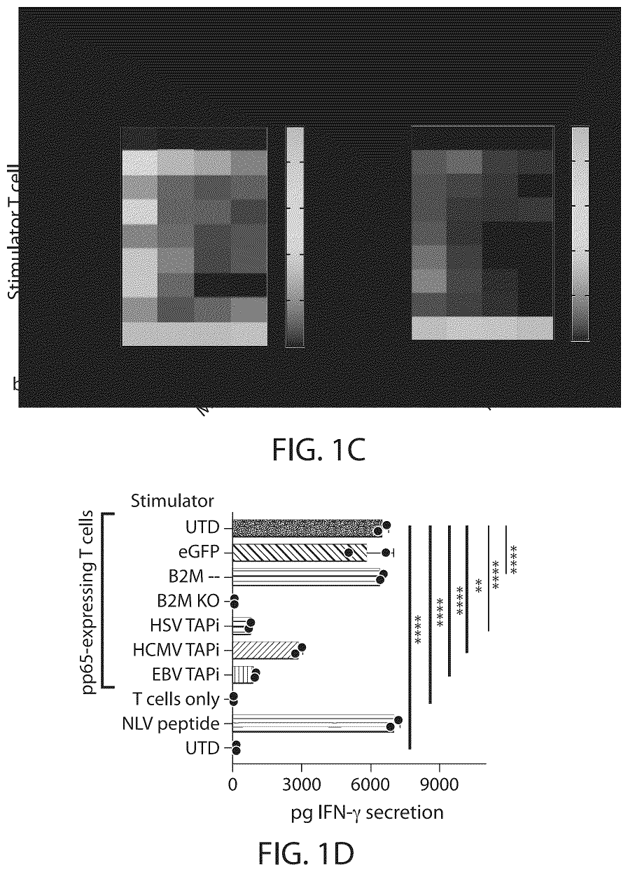

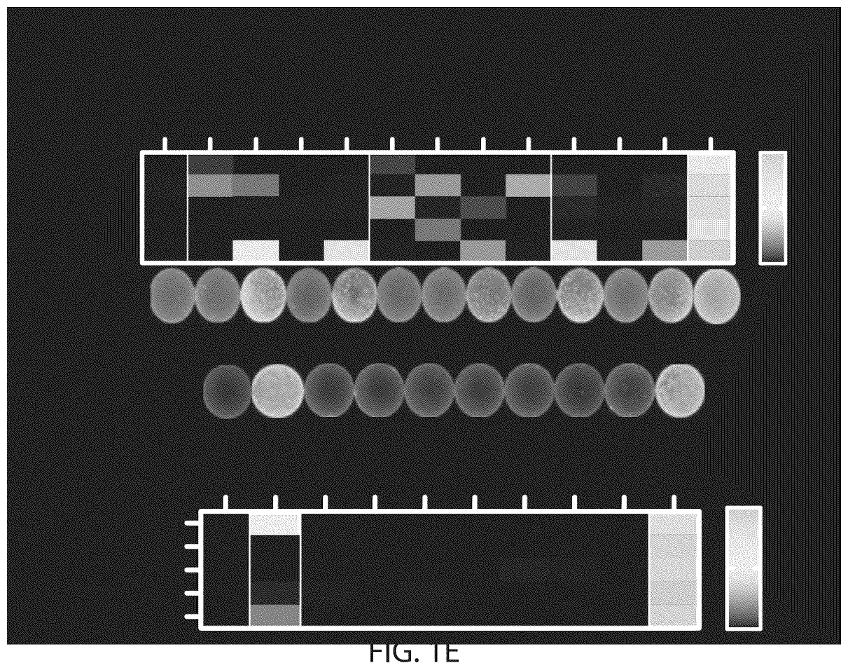

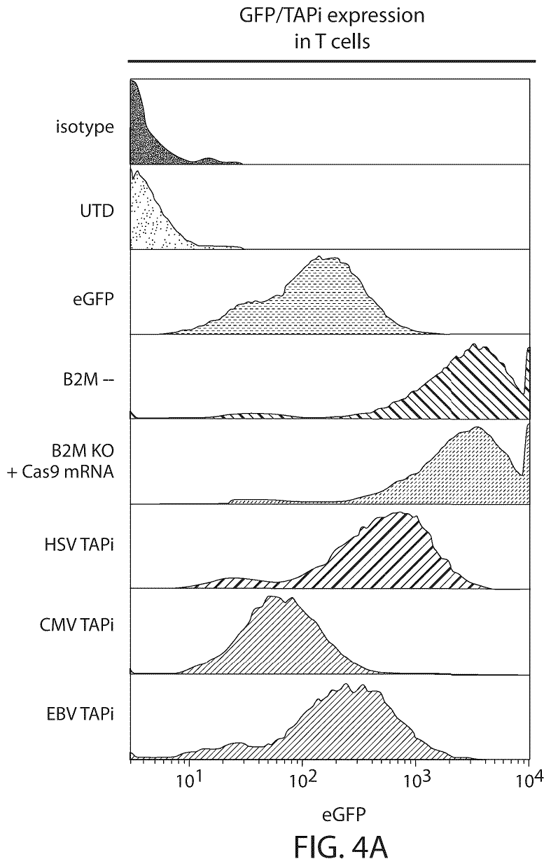

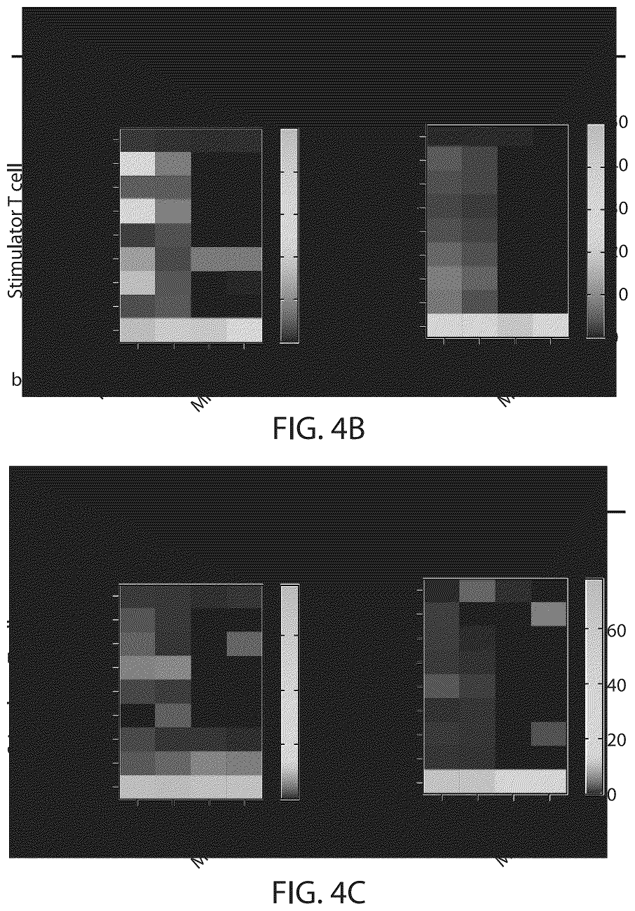

Herpesviruses are a class of chronic viruses that infect various human cells and manage to evade T cell immunity. Herpesvirus have convergently evolved to encode small proteins that inhibit TAP¹³, a protein required for transporting cytoplasmic peptides across the endoplasmic reticulum and loading them for presentation on MHC Class I molecules at the cell surface. Cells that naturally or experimentally lack expression of functional TAP complexes show a dramatic reduction in surface MHC I levels which substantially reduces their sensitivity to CD8⁺ T cells¹⁴. The disclosure is directed, in part, to the discovery that forced expression of viral TAP inhibitors (TAPi) reduces MHC I expression in gene-modified cells, thereby preventing cell-mediated immune responses to foreign transgenes. To test if expression of herpesvirus TAPi reduced surface MHC I expression in primary T cells, bicistronic lentiviral constructs were generated to express Herpes Simplex virus (HSV) ICP47, Human Cytomegalovirus (HCMV) US6, or Epstein-Barr virus (EBV) BNLF2a TAPi along with a fluorescent reporter eGFP as a marker of transduction (FIG. 1A). Lentiviral constructs expressing sgRNA for 0-2-microglobulin (β2M) with or without electroporated with Cas9 mRNA were used as a positive control (β2M KO) or negative control (β2M−−). Primary human T cells consistently expressed eGFP upon transduction with the lentiviral vectors (FIG. 4A) and TAPi-transduced cells had reduced levels of surface MHC I without affecting MHC Class II upregulation upon activation (FIG. 1B).

Since MHC I expression inhibits targeting by NK cells¹⁵, the impact of MHC I downregulation on susceptibility to NK cell killing was investigated. Like previous reports¹², β2M KO T cells were susceptible to autologous NK cell lysis and induced NK cell degranulation, as measured by CD107a expression. Importantly, T cells expressing viral TAPi did not significantly trigger NK cell lysis or degranulation compared to untransduced (UTD) T cells (FIG. 1B). Similarly, MHC I expression mediates allogeneic T cell responses due to mismatch between MHC and TCR. To measure the allogeneic response of TAPi expressing T cells, a mixed lymphocyte reaction (MLR) was performed. Transduced T cells were incubated with autologous or allogeneic labeled responder T cells in the presence or absence of MHC I and II blocking antibodies. Responder T cell activation was measured by proliferation (FIG. 1C) and changes in CD69 and CD25 expression (FIGS. 4B-4C). T cells transduced with viral TAPi, especially EBV BNLF2a, induced less allogeneic responder T cell activation, which was further decreased by MHC I and/or MHC II blockade.

Next, the functional ability of TAPi-expressing T cells to present cytoplasmic antigens was tested by assessing the presentation of a peptide derived from the highly immunogenic HCMV pp65 protein. The immunogenic NLV peptide is presented on the HLA-A*02:01 allele and drives NLV-specific CD8⁺ T cells to secrete IFNγ¹⁶. Lines of NLV-specific “responder T cells” were first generated by serial stimulation of PBMC derived from HLA-A*0201 healthy donors who had evidence of CMV-specific memory responses. Then, a panel of “stimulator T cells” were generated, derived from the same healthy donors, which were untransduced (UTD) or transduced with the constructs as shown, including 3 different viral TAPi. Co-cultures of “stimulator cells” with the “responder cells” demonstrated that viral TAPi expression, especially when derived from HSV or EBV, impeded antigen presentation, as shown by reduced IFNγ secretion in “responder T cells” (FIG. 1D). Despite reduced antigen presentation, the use of a viral protein to knock down the MHC I raises the possibility of an immune response to its sequence. To measure the immunogenicity of viral TAPi transduction in T cells, normal donors were identified with pre-existing cellular immunity to the respective TAPi viruses. PBMCs from normal donors were screened with peptides known to be immunogenic and originating from HCMV, EBV or HSV in an IFNγ ELISpot assay. T cells from normal donors with a detectable cellular response those viruses were then transduced with viral TAPi from the same virus and incubated with autologous CD8⁺ T cells. CD8 T cell activation was measured by IFNγ ELISpot (FIG. 1E). While T cells from an HCMV-responsive donor were activated in response to transduction with HCMV pp65, they did not respond to transduction with the CMV TAPi. Similarly, HSV- and EBV-responsive donors did not produce IFNγ in response to HSV or EBV TAPi, indicating that these viral TAP inhibitors do not elicit T cell responses, despite having demonstrated responsiveness to other known immunogenic sequences from the same viruses.

Taken together, the results showed that primary T cells expressing HSV, EBV, or HCMV TAPi efficiently prevented cell surface expression of MHC I molecules, thereby limiting killing by NK cells and mitigating allogeneic responses. The few remaining MHC I molecules on the cell surface were not sufficient to mount T cell activation in response to immunogenic peptides or peptides derived from the viral TAP inhibitor itself.

Expression of shRNA Targeting CIITA Results in Decreased Cell Surface Levels of MHC Class II and can be Co-Expressed with EBV TAPi to Reduce Both MHC Class I and II at the Cell Surface

Activated human T cells express high levels of MHC class II molecules, which may also trigger rejection and antigen cross-presentation of gene-modified cells^{4,17}. MHC Class II expression was previously reduced by targeting CIITA, the main regulatory factor that controls the transcription of MHC II genes¹⁸.

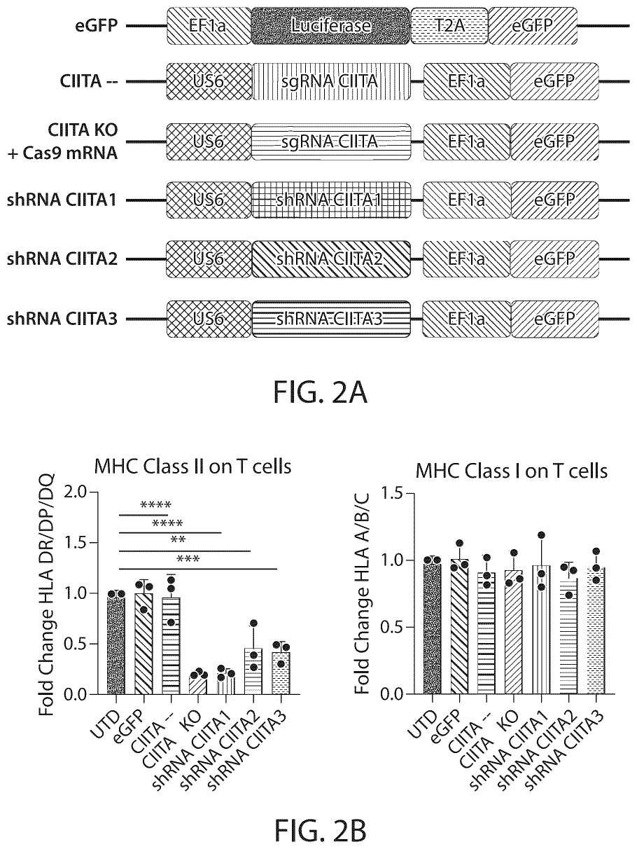

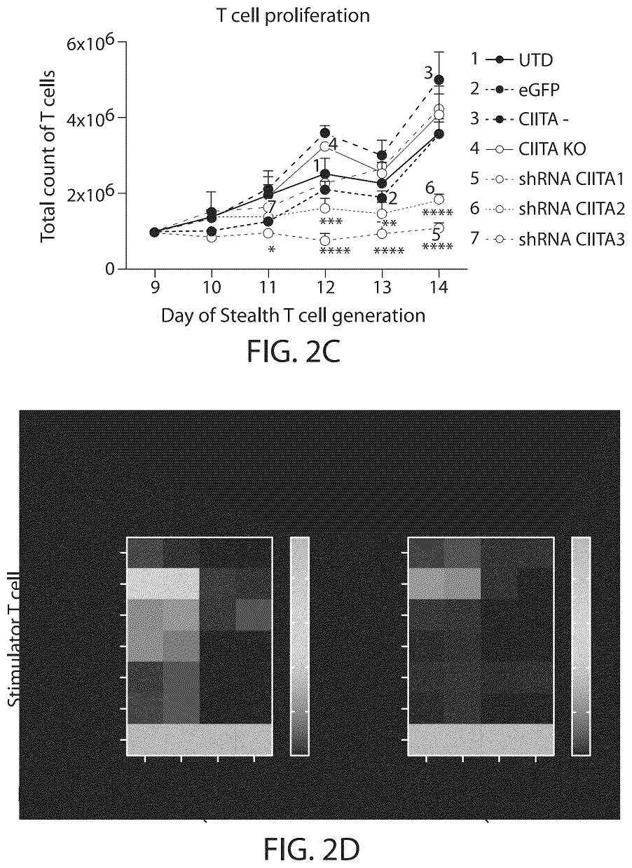

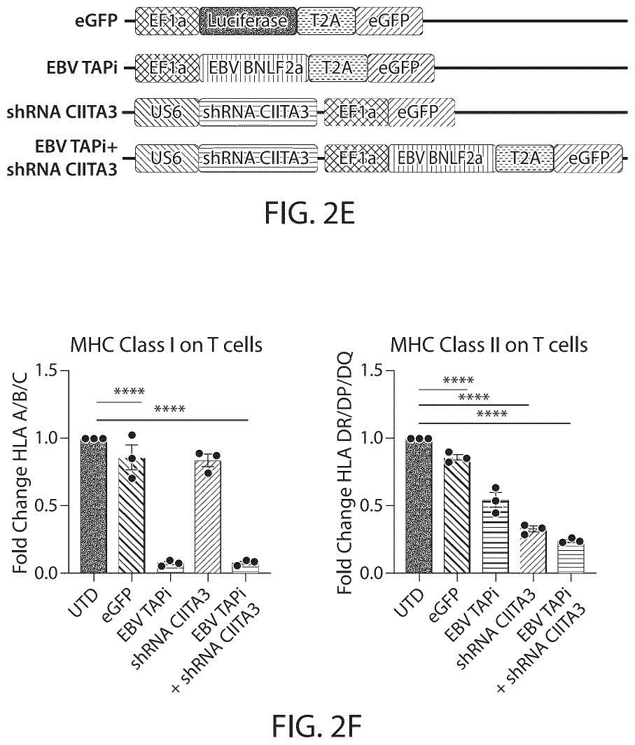

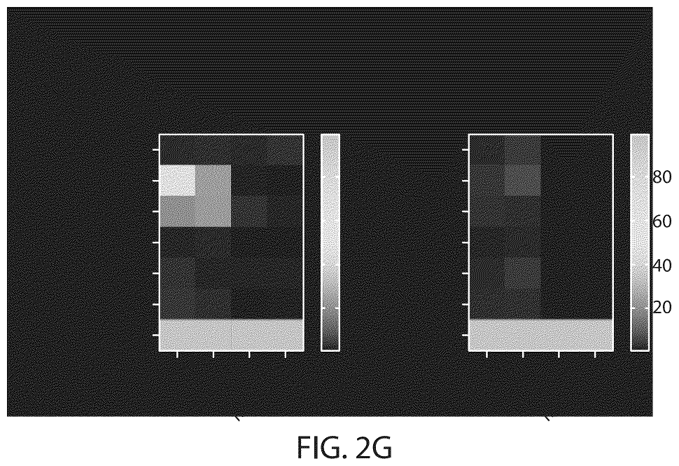

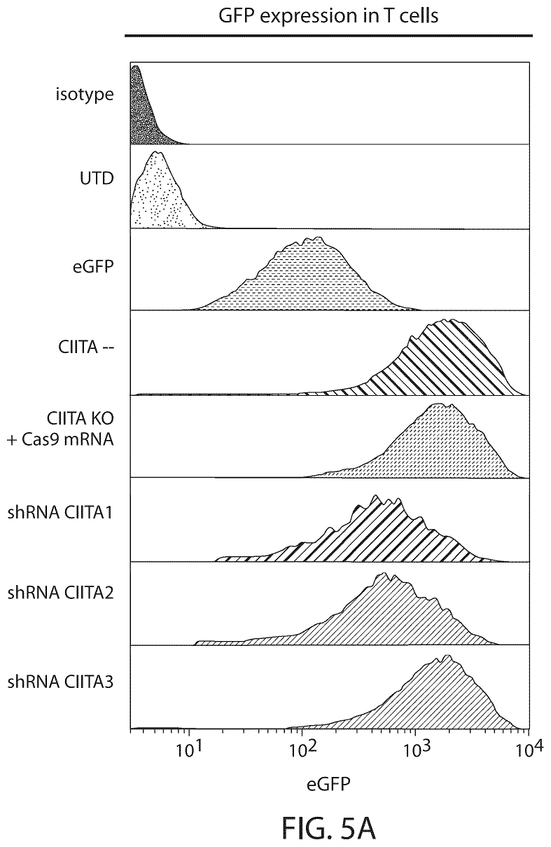

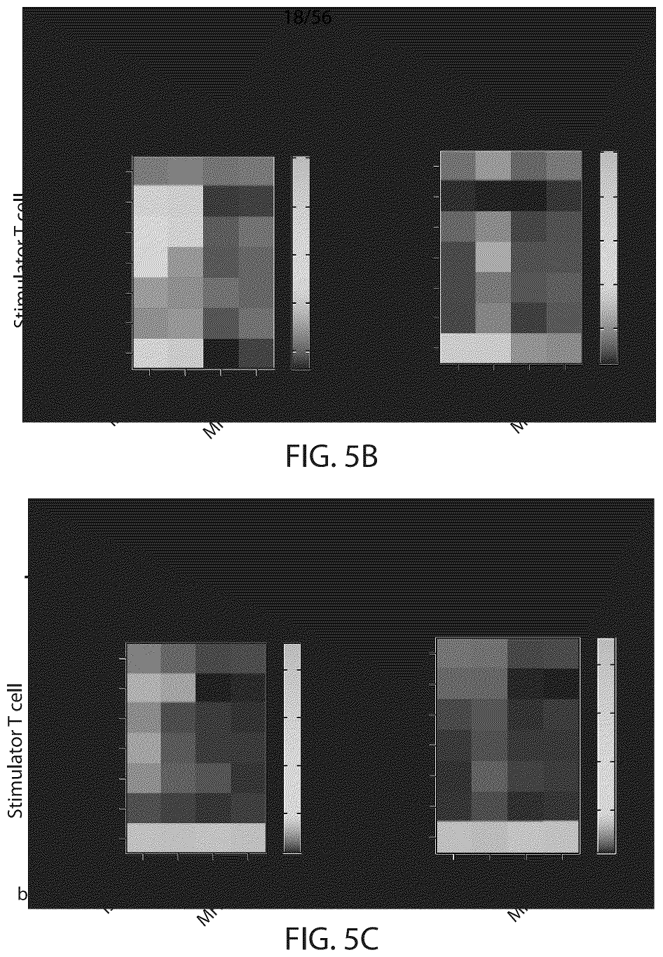

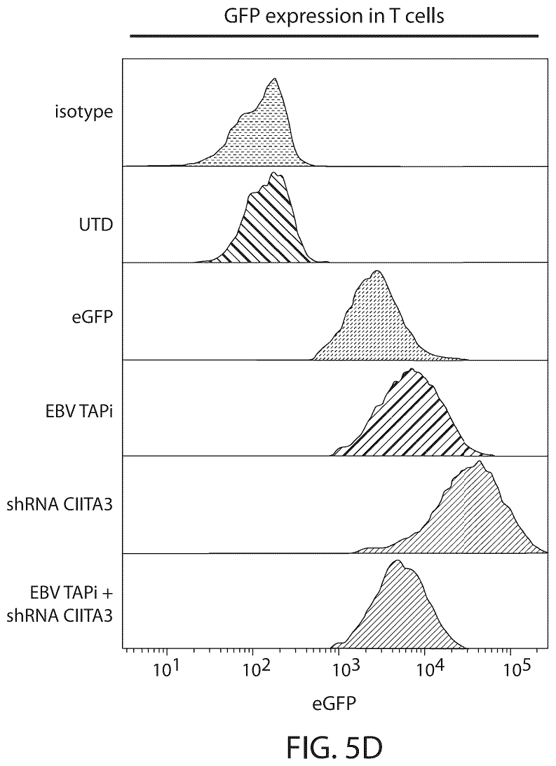

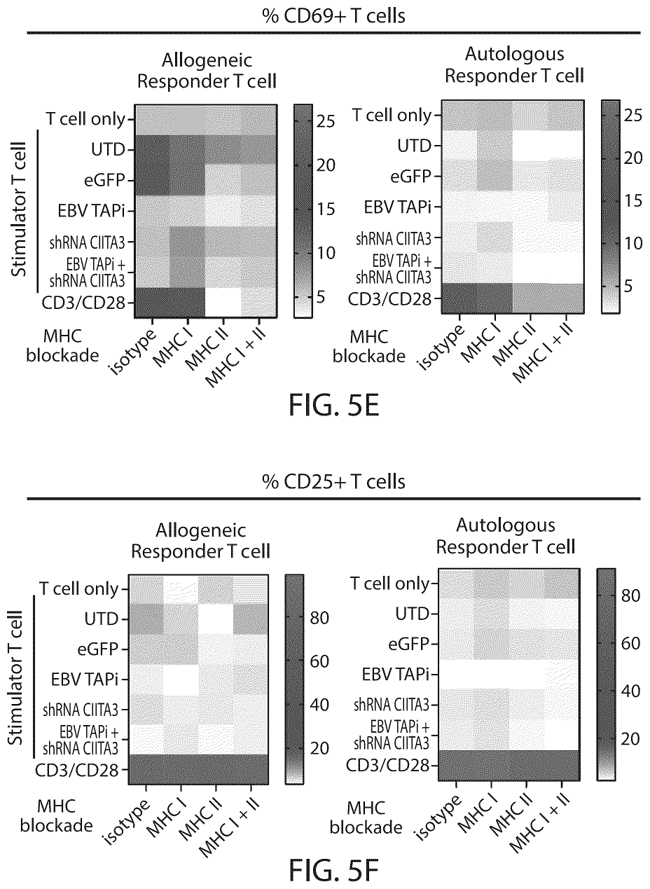

To avoid the use of gene-editing and double-strand breaks, shRNA targeting CIITA was encoded into the lentiviral vectors (FIG. 2A), and a panel of shRNA sequences was used as well as a comparison of the shRNA vectors to gene knockout of CIITA with CRISPR/Cas9. Transduction efficiency was measured based on eGFP expression (FIG. 5A); it was noted that primary human T cells transduced with CIITA-targeting shRNA had reduced cell surface expression of MHC II, comparable to CIITA KO, without affecting MHC I expression (FIG. 2B). However, only shRNA CIITA3 reduced MHC II expression without compromising T cell proliferation (FIG. 2C). Measurements of proliferation of responder allogeneic or autologous T cells in a mixed lymphocyte reaction (MLR) demonstrated that shRNA-mediated knockdown of CIITA reduced responder T cell proliferation (FIG. 2D FIGS. 5B-5C). Next, the MHC I and II downregulation strategies were combined by including both EBV TAPi and shRNA CIITA3 into one lentiviral vector (FIG. 2E). When transduced into primary human T cells, the combined EBV-TAPi/shRNA-CIITA3 vector reduced MHC I and II expression (FIG. 2F) and reduced proliferative responses in MLRs (FIG. 2G; FIG. 5D-E). These results demonstrated that gene-modified primary T cells can successfully evade cellular immune responses by MHC class I and II downregulation strategies as proposed herein, creating “stealth” T cells.

Stealth-Enabled αCD19 CAR T Cells are Functional and Capable of Evading CAR-Mediated Immune Recognition by T Cells from Patients Who Received a Single or Double Infusion of αCD19 CAR T Cells.

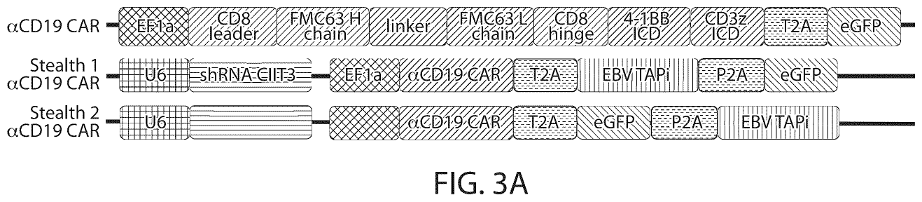

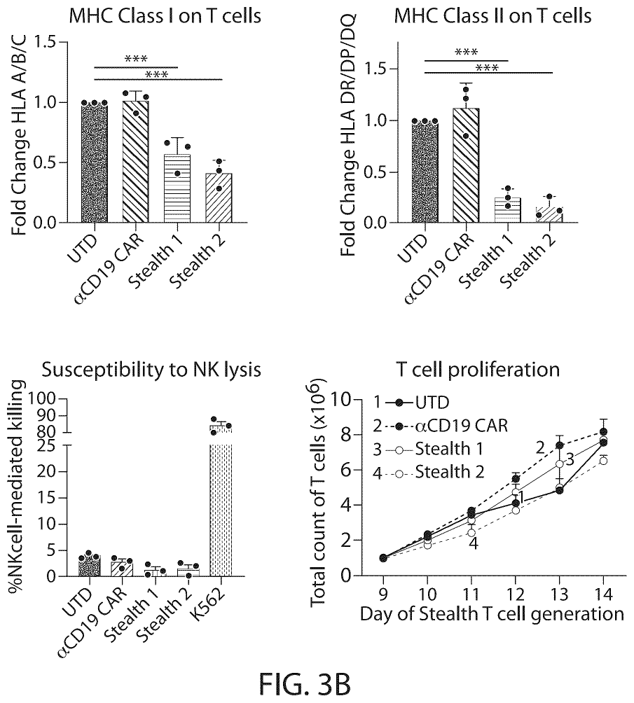

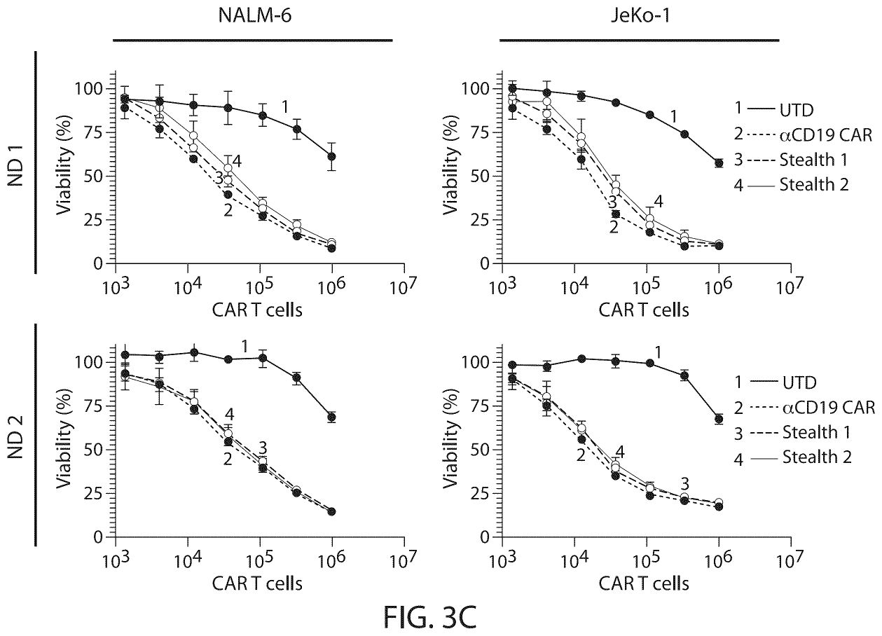

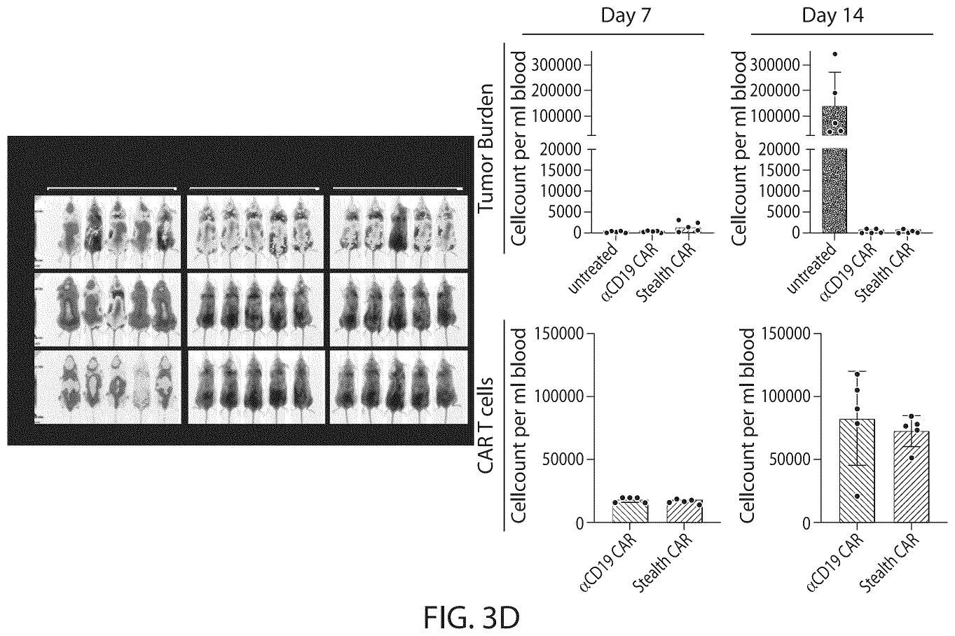

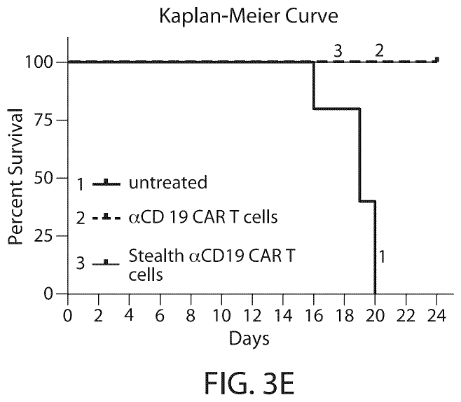

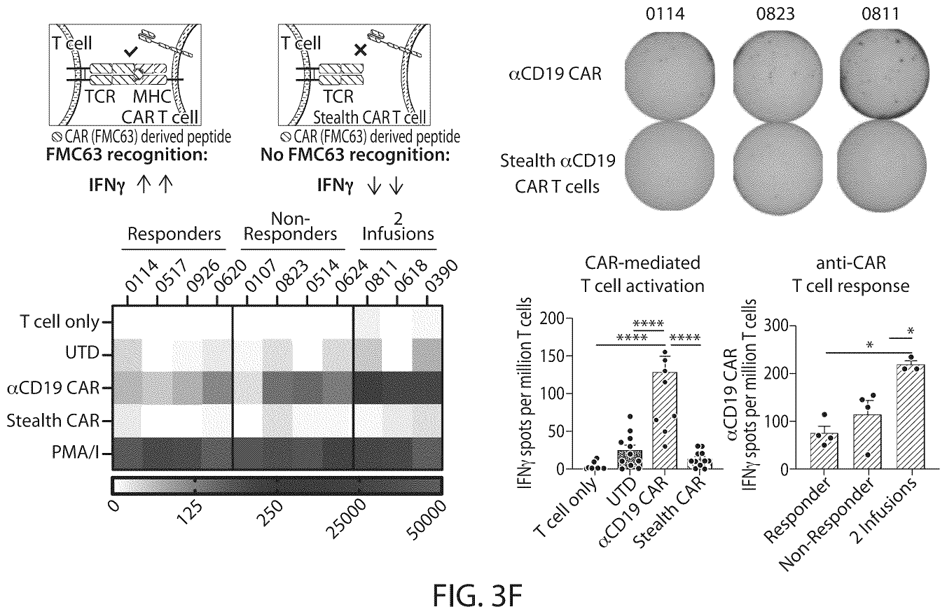

Autologous CAR T cells based on FMC63-based αCD19 single-chain scFv can elicit T cell responses against the murine scFv-fractions of the CAR in the patients^{3,4}. Thus, the stealth strategy proposed herein was tested in the context of these CARs to verify retention of anti-tumor efficacy and avoidance of cellular immunity. Two stealth FMC63-based αCD19 CAR were generated, alternating the sequence position of the EBV TAPi and the eGFP marker (FIG. 3A). Both stealth αCD19 CAR-T cells had reduced MHC I and II molecules on their cell surface compared to the αCD19 CAR alone. Interestingly, this reduction did not increase NK cell cytotoxicity, and proliferation of the CAR-T cells was unchanged compared to the untransduced T cells (FIG. 3B).

The stealth αCD19 CAR-T cells also maintained their ability to target tumor cells in vitro and in vivo. When co-incubated with luciferase-expressing acute lymphoblastic leukemia (ALL) NALM6 cells or Mantle cell lymphoma JeKo-1 cells, stealth αCD19 CAR-T cells reduced tumor cell viability to the same extent as αCD19 CAR-T cells (FIG. 3C). Due to its slight advantage in MHC I downregulation, the configuration of stealth2 αCD19 CAR-T cells was selected and further studied in an in vivo NSG mouse model with ALL NALM6 cells. After tumor engraftment, mice were left untreated or injected with αCD19 CAR-T cells with or without stealth technology and assessed for CAR-T cell expansion by blood draws and tumor clearance by bioluminescence (BLI) (FIG. 3D). Both αCD19 CAR-T cells and stealth αCD19 CAR-T cells expanded similarly in the blood as observed on day 7 and day 14 by flow cytometric assessment of GFP+ CD3+ cells. Tumor cells, GFP+CD3− NALM6 cells, were found to be absent on both timepoints, whilst a large expansion was found in the untreated group. This was further confirmed by BLI imaging. Treatment of engrafted NALM6 with αCD19 CAR-T cells and- stealth αCD19 CAR-T cells showed comparable tumor clearance, whilst in untreated mice the luciferase-expressing NALM6 cells vastly expanded. Kaplan-Meier survival curves demonstrated no difference in survival of mice treated with αCD19 CAR-T cells with or without the additional stealth sequences in the vectors (FIG. 3E). In summary, stealth αCD19 CAR-T retained their ability to recognize and clear CD19-expressing cells in both in vitro and in vivo tumor models.

Finally, the ability of the proposed stealth technology to avoid antigen presentation of immunogenic CAR sequences was tested. Eleven patients who had received autologous FMC63-based CARs either once or twice were identified, and fresh αCD19 CAR-T cells with or without the stealth technology were generated from their 3-month post-infusion PBMC. Four of the patients had initial responses of their tumor to their CAR T cell product, four had tumors that did not respond to CAR T cells, and three had received a second infusion of CAR T cells due to tumor progression after the first infusion. To assess whether T cells from these patients could be activated by their autologous T cells expressing the FMC63-based αCD19 CAR, the freshly made αCD19 CAR-T cells with or without stealth technology were used as “stimulators” and co-cultured these with autologous “responder” untransduced T cells in an IFNγ ELISpot assay (FIG. 3F). Responder T cells became activated in the presence of FMC63-based αCD19 CAR-T cell products, but not UTD cells or stealth αCD19 CAR-T cells. Activation of responder T cells was particularly high in those subjects who had received 2 infusions of FMC63-based CAR T cells, and in 3 of the 4 non-responders. These data provide evidence that multiple infusions can increase anti-CAR immunity in patients, and that a fraction of non-responders have robust rejection of their autologous αCD19 CAR-T cells.

### Discussion

In summary, the results demonstrated that the combined expression of EBV TAPi BNLF2a and shRNA targeting CIITA effectively reduces the MHIC expression and antigen presentation, and incorporation of these sequences into a lentiviral vector has potential use to evade autologous and allogeneic cellular immunity. Evasion of endogenous T cell-mediated rejection can be especially valuable in αCD19 CAR-T cell therapy where initial expansion and persistence is associated with durable remission^{19,20}. Because αCD19 CAR-T cells efficiently eliminate the B cell-lineage, humoral immunity to αCD19 CAR-T cell therapy is limited, which further enhances the impact of avoiding T cell immunity in this setting^{4,21} More generally, this stealth technology can be applied in any setting that employs gene-modified cells where either the transgene, junctional sequences, or the cell types are not autologous and where avoidance of early rejection can enhance the desired therapeutic effects. See, e.g., references 4, 6, and 22.

The results further demonstrated that stealth CAR-T cells evade anti-CAR responses originating from the FMC63-based αCD19 CAR, while avoiding NK cell activation due to loss of MHC I on the cell surface. Furthermore, an increased CAR-reactive T cell response was found in patients who received multiple FMC63-based αCD19 CAR-T cell infusions.

### Methods

### Mice and Cell Lines

NSG mice were purchased from Jackson Laboratory and bred under pathogen-free conditions at the Center for Comparative Medicine at MGH. All experiments were performed according to protocols approved by the Massachusetts General Hospital institutional Animal Care and Use Committee. Where indicated, cell lines were transduced and expanded after clonal selection to express click beetle green (C(BG luciferase and enhanced GFP. 1-HEKT cells, NALM-6 (ALL), JeKo-I (MCL) and K562 (CML) were purchased from the American Type Culture Collection and maintained under conditions as outlined by the supplier.

### (Stealth) CAR T Cell Production

Human T cells were purified from anonymous human healthy donor leukapheresis product (Stem Cell Technologies) purchased from the MGH blood bank under an Institutional Review Board-exempt protocol. T cell from patients treated with axicabtagene ciloleucel or tisagenlecleucel at MGH were collected on an IRB-approved protocol with written informed consent; PMBC from one subject treated at Seattle Cancer Care Alliance with two infusions of autologous FMC63-based CAR T cells were provided by Dr. Turtle and collected with written informed consent. Cells were transduced with lentivirus corresponding to various second-generation CAR-T-cell constructs. In brief, bulk human T cells were activated on day 0 using CD3/CD28 Dynabeads (Life Technologies) and cultured in RPMI 1640 medium with GlutaMAX and HEPES supplemented with 10% FBS and 20 IU ml⁻¹ recombinant human IL-2. Lentiviral transduction of cells was performed on day 1 and on day 5 CD3/CD28 Dynabeads were removed and, if applicable, the T cells were electroporated with Cas9 mRNA. If the T cells needed to be sorted, the T cells were sorted to purity on day 8 using the eGFP marker and left to expand until day 14, to be subsequently transferred to storage in liquid nitrogen. When unsorted CAR T cells were used, CAR-T cells were normalized for transduction efficiency using untransduced but cultured and activated T cells from the same donor and expansion.

### Cytotoxicity Assay

To assess the cytotoxicity of CAR T cells towards target cells, CAR T cells were incubated with luciferase-expressing tumor targets at indicated E/T ratios for 24h. Remaining luciferase activity was subsequently measured with a Synergy Neo2 luminescence microplate reader (Biotek). To assess the cytotoxicity of NK cells towards stealth or CAR T cells, NK cells were purified from blood or frozen PBMCs (Stein Cell Technologies) and primed with 20 IU ml⁻¹ recombinant human IL-2 before co-incubation with their respective target cells stained with CFSE (Life Technologies). After 3h of co-incubation CD107a antibodies were added and the assay was left to incubate for another hour. After a total of 4 h, cells were centrifuged, resuspended with dead/alive marker SYTOXred (Life Technologies), and assessed by flow cytometer for target cell viability and NK cell degranulation.

### ELISpot Assay

Plates with Immobilon-P membrane (Millipore) were activated with 35% Ethanol for 30 seconds, washed with PBS and incubated overnight with PBS containing anti-human IFNγ antibody (Clone NIB42, Biolegend). The next day, the plate was blocked with PBS containing 1% BSA and 5×10⁵ PBMCs or 2×10⁵ T cells were co-incubated with respective peptides, antigens, or stimulants. After 24 h, the plate was washed with PBS containing 0.05% Tween-20 and incubated overnight with PBS containing biotinylated anti-human IFNγ antibody (Clone 4S.B3, Biolegend) as detection antibody. After washing with PBS containing 0.05% Tween-20, the plate is incubated for 2 h with avidin-HRP (Biolegend), developed using the BD Elispot AEC Substrate set and analyzed with ImmunoSpot Reader systems. All antibodies were used according to the manufacturers' recommendation.

### ELISA

Interferon γ from supernatants was measured following an overnight co-incubation of NLV responder T cells with target at a E:T ratio of 1:5 using Human DuoSet ELISA kits (R&D systems).

### Flow Cytometry

Generally, cells were stained in the dark for 30 min at 4° C. and washed twice with RPMI before analysis. SYTOXRed or SYTOXBlue (Life Technologies) were added as dead/alive marker and singlet discrimination was performed on both the FSC and SSC detectors. The following antibodies targeting their respective antigens were used according to the manufacturers' recommendations in combination with their respective isotype control: CD4 (SK3, Biolegend), CD8 (SKi, Biolegend), CD3 (OKT3, Biolegend), CD25 (BC96, Biolegend), CD69 (FN50, Biolegend), HLA-A/B/C (W6/32, Biolegend), HLA-DR/DP/DQ (T039, Biolegend), CD107a (H4A3, Biolegend), murine erythroid cells (TER-119, Biolegend), murine Ly6G/6C (RB6-8C5, Biolegend), murine CD11b (M1/70, Biolegend) and murine NK1.1 (PK136, Biolegend). Analysis was performed by FlowJo software (BD Biosciences).

### Mixed Lymphocyte Reaction (MLR) Assay

Stealth or CAR T cells were stained with CFSE (Life Technologies), whilst autologous or allogeneic T cells were stained with CellTrace Violet (Life Technologies) before being co-incubated at a 4:1 ratio in the presence of 20 IU ml⁻¹ recombinant human IL-2 and either isotype or MHC I (W6/32, Biolegend) or MHC II (T039, Biolegend) or both MHC I and II blocking antibodies. Fresh IL-2 was added every other day and the T cells were pulsed with new stealth T cells and blocking antibodies on day 7 and 14. On day 16, T responder cells were stained and assessed by FCM for cell division and activation markers CD69 and CD25.

### In Vivo Study

Luciferized NALM-6 cells were harvested in logarithmic growth phase, washed twice with PBS, and counted before injecting these tumor cells (1×10⁶ NALM-6 cells per mouse) in NSG mice by tail vein. Presence of the tumor was confirmed 3 days later by bioluminescence, at which time the mice were treated by an injection of 2×10⁶ CAR T cells in the tail vein. Tumor progression was then longitudinally evaluated by bioluminescence emission using an Ami HT optical imaging system (Spectral Instruments) following intraperitoneal substrate injection. At day 7 and day 14, the blood of the mice was collected by cheek punch and analyzed by FCM for presence of NALM-6 and CAR T cells per microliter blood.

### Stealth CAR Design

DNA constructs were synthesized and cloned into a second-generation lentiviral backbone under the regulation of a human EF-1α promoter for protein translation and/or a human U6 promoter for RNA transcription. The sequences for EBV BNLF2a, HSV ICP47 and HCMV US6TAPi were synthesized and combined with eGFP by means of an 2A self-cleaving peptide. The shRNA targeting CIITA were designed with software of Dharmacon and the Whitehead institute and combined in a plasmid expressing eGFP by EF-1α promoter. Similarly, vectors with CRISPR/Cas9 guides for β2M and CIITA and eGFP expression were constructed. The lentiviral vector expressing the combination of shRNA CIITA3, EBV BNLF2a and eGFP was also constructed. For CAR constructions, plasmid expressing the FMC63-based anti-CD19 CAR were constructed in combination with expression of EBV BNLF2a and shRNA targeting CIITA.

### Statistical Methods

All statistical analyses were performed with GraphPad Prism 9 software. Data were presented as means±s.e.m. with statistically significant differences determined by tests as indicated in figure legends.

### References in Example 1

1. Locke, F. L., et al. Axicabtagene Ciloleucel as Second-Line Therapy for Large B-Cell Lymphoma. N Engl J Med (2021).
2. Bishop, M. R., et al. Second-Line Tisagenlecleucel or Standard Care in Aggressive B-Cell Lymphoma. N Engl J Med (2021).
3. Turtle, C. J., et al. CD19 CAR-T cells of defined CD4+:CD8+ composition in adult B cell ALL patients. J Clin Invest 126, 2123-2138 (2016).
4. Wagner, D. L., et al. Immunogenicity of CAR T cells in cancer therapy. Nat Rev Clin Oncol 18, 379-393 (2021).
5. Jensen, M. C., et al. Antitransgene rejection responses contribute to attenuated persistence of adoptively transferred CD20/CD19-specific chimeric antigen receptor redirected T cells in humans. Biol Blood Marrow Transplant 16, 1245-1256 (2010).
6. Lamers, C. H., et al. Immune responses to transgene and retroviral vector in patients treated with ex vivo-engineered T cells. Blood 117, 72-82 (2011).
7. Depil, S., Duchateau, P., Grupp, S. A., Mufti, G. & Poirot, L. ‘Off-the-shelf’ allogeneic CAR T cells: development and challenges. Nat Rev Drug Discov 19, 185-199 (2020).
8. Tuladhar, R., et al. CRISPR-Cas9-based mutagenesis frequently provokes on-target mRNA misregulation. Nat Commun 10, 4056 (2019).
9. Allogene. Allogene Therapeutics Reports FDA Clinical Hold. (2021).
10. Choi, B. D., et al. CRISPR-Cas9 disruption of PD-1 enhances activity of universal EGFRvIII CAR T cells in a preclinical model of human glioblastoma. Journal for immunotherapy of cancer 7, 304 (2019).
11. Biosciences, P. (2021).
12. Kagoya, Y., et al. Genetic Ablation of HLA Class I, Class II, and the T-cell Receptor Enables Allogeneic T Cells to Be Used for Adoptive T-cell Therapy. Cancer Immunol Res 8, 926-936 (2020).
13. Verweij, M. C., et al. Viral inhibition of the transporter associated with antigen processing (TAP): a striking example of functional convergent evolution. PLoS Pathog 11, e1004743 (2015).
14. Goldsmith, K., Chen, W., Johnson, D. C. & Hendricks, R. L. Infected cell protein (ICP)47 enhances herpes simplex virus neurovirulence by blocking the CD8⁺ T cell response. J Exp Med 187, 341-348 (1998).
15. Vivier, E., Tomasello, E., Baratin, M., Walzer, T. & Ugolini, S. Functions of natural killer cells. Nat Immunol 9, 503-510 (2008).
16. Khan, N., Cobbold, M., Keenan, R. & Moss, P. A. Comparative analysis of CD8⁺ T cell responses against human cytomegalovirus proteins pp65 and immediate early 1 shows similarities in precursor frequency, oligoclonality, and phenotype. J Infect Dis 185, 1025-1034 (2002).
17. Costantino, C. M., Spooner, E., Ploegh, H. L. & Hafler, D. A. Class II MHC self-antigen presentation in human B and T lymphocytes. PLoS One 7, e29805 (2012).
18. Holling, T. M., van der Stoep, N., Quinten, E. & van den Elsen, P. J. Activated human T cells accomplish MHC class II expression through T cell-specific occupation of class II transactivator promoter III. J Immunol 168, 763-770 (2002).
19. Maude, S. L., et al. Chimeric antigen receptor T cells for sustained remissions in leukemia. N Engl J Med 371, 1507-1517 (2014).
20. Porter, D. L., et al. Chimeric antigen receptor T cells persist and induce sustained remissions in relapsed refractory chronic lymphocytic leukemia. Sci Transl Med 7, 303ra139 (2015).
21. Elahi, R., Heidary, A. H., Hadiloo, K. & Esmaeilzadeh, A. Chimeric Antigen Receptor-Engineered Natural Killer (CAR NK) Cells in Cancer Treatment; Recent Advances and Future Prospects. Stem Cell Rev Rep 17, 2081-2106 (2021).
22. Jan, M., et al. Reversible ON- and OFF-switch chimeric antigen receptors controlled by lenalidomide. Sci Transl Med 13(2021).

### Example 2. Additional Results Demonstrating Expression of Viral TAP Inhibitors in Primary T Cells Results in Decreased Cell Surface Levels of MHC Class I

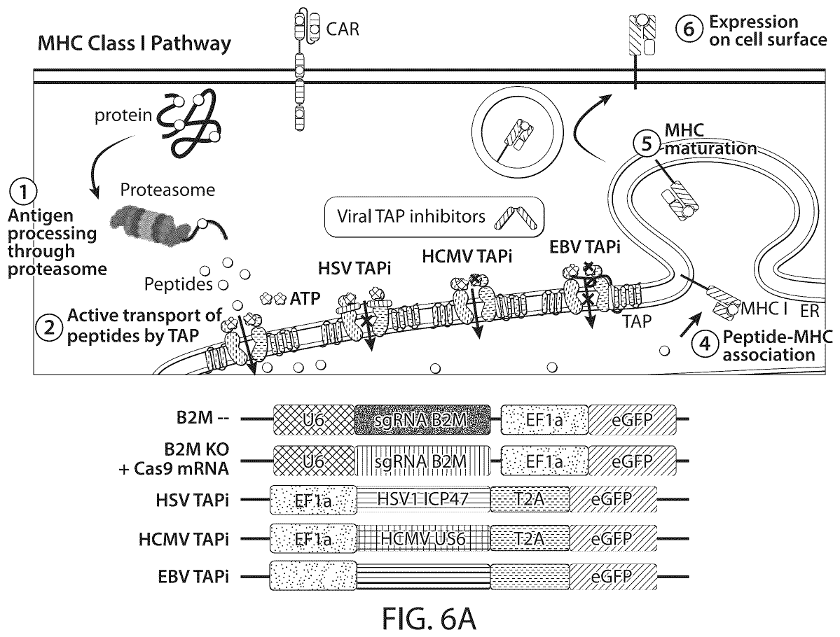

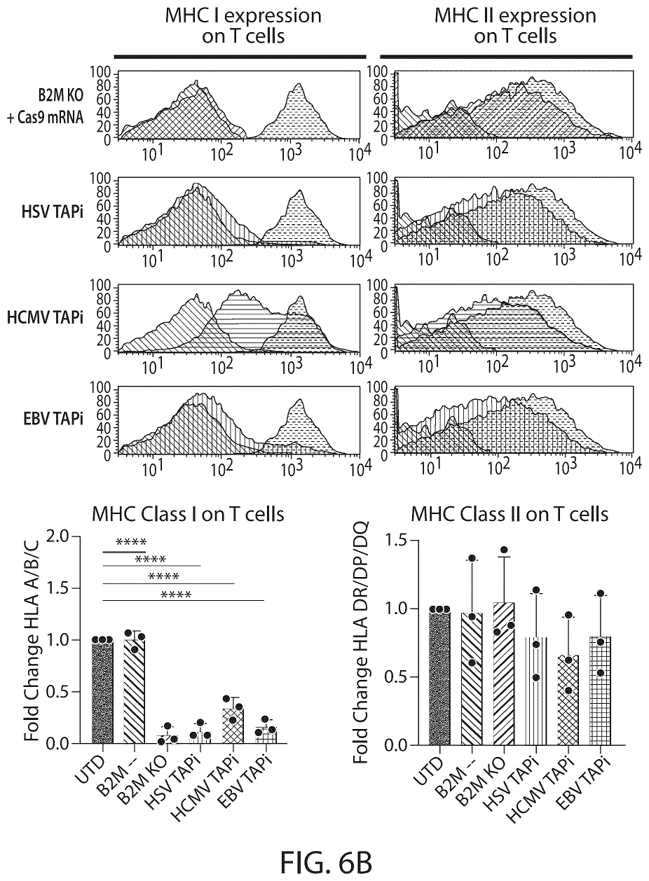

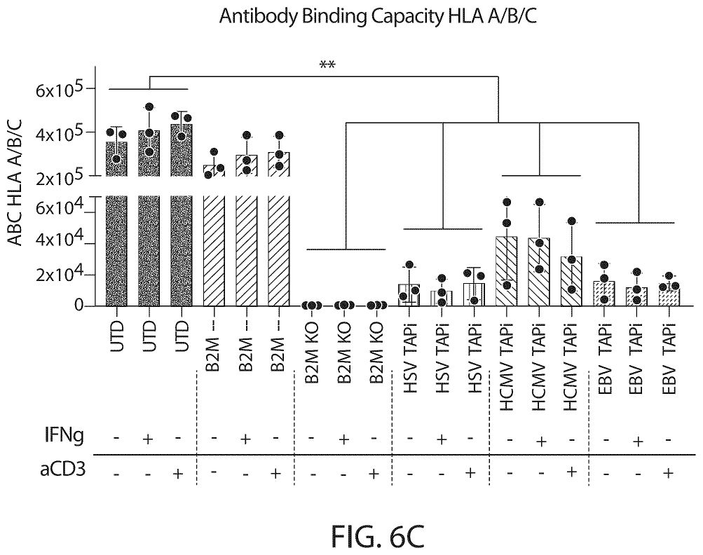

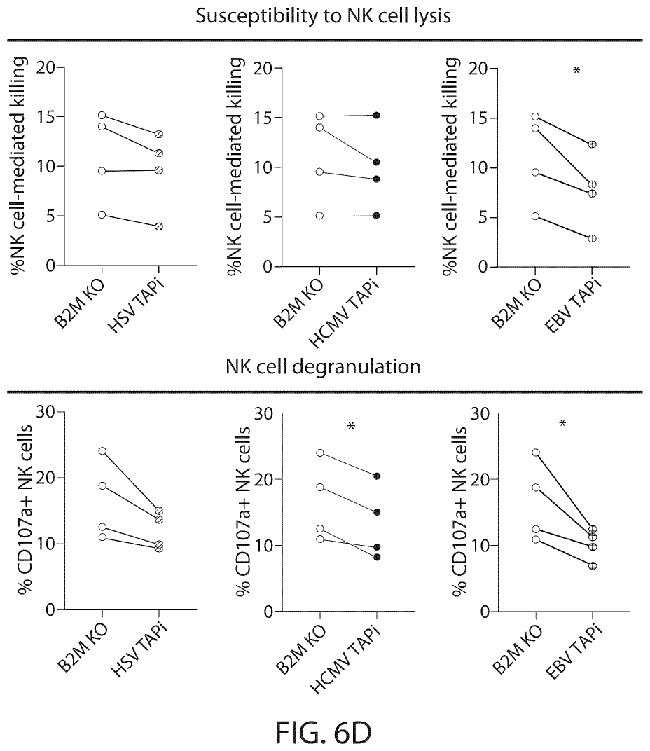

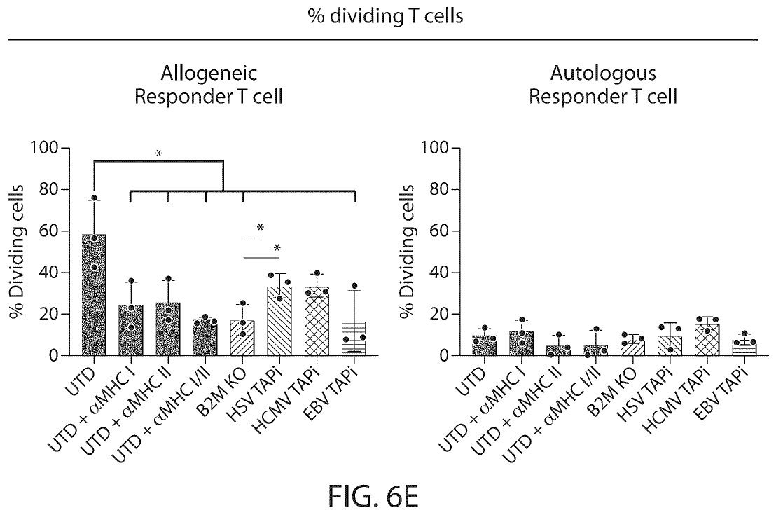

Herpesviruses have convergently evolved to encode small proteins that inhibit TAP²⁹, a protein required for transporting cytoplasmic peptides across the endoplasmic reticulum and loading them for presentation on MHC Class I molecules at the cell surface. Cells that naturally or experimentally lack expression of functional TAP complexes show a dramatic reduction in surface MHC I levels, substantially reducing their sensitivity to CD8⁺ T cells.³⁰ It was hypothesized that forced expression of viral TAP inhibitors (TAPi) would reduce MHC I expression in gene-modified cells, thereby preventing cell-mediated immune responses to foreign transgenes. To test if expression of herpesvirus TAPi reduced surface MHC I expression in primary T cells, bicistronic lentiviral constructs were generated to express Herpes Simplex virus (HSV) ICP47, Human Cytomegalovirus (HCMV) US6, or Epstein-Barr virus (EBV) BNLF2a TAPi along with a fluorescent reporter eGFP as a marker of transduction (FIG. 6A). Lentiviral constructs expressing sgRNA for β-2-microglobulin (β2M), w/o electroporated with Cas9 mRNA, were used as a positive control (β2M KO) or negative control (β2M−−). At similar transduction efficiencies, TAPi-transduced cells had reduced levels of surface MHC I without affecting MHC Class II upregulation upon activation (FIG. 6B). Viral TAP inhibitors reduced total surface MHC I levels by at least one log-fold, which was maintained upon additional stimulation by IFNγ or αCD3-antibody (FIG. 6C).

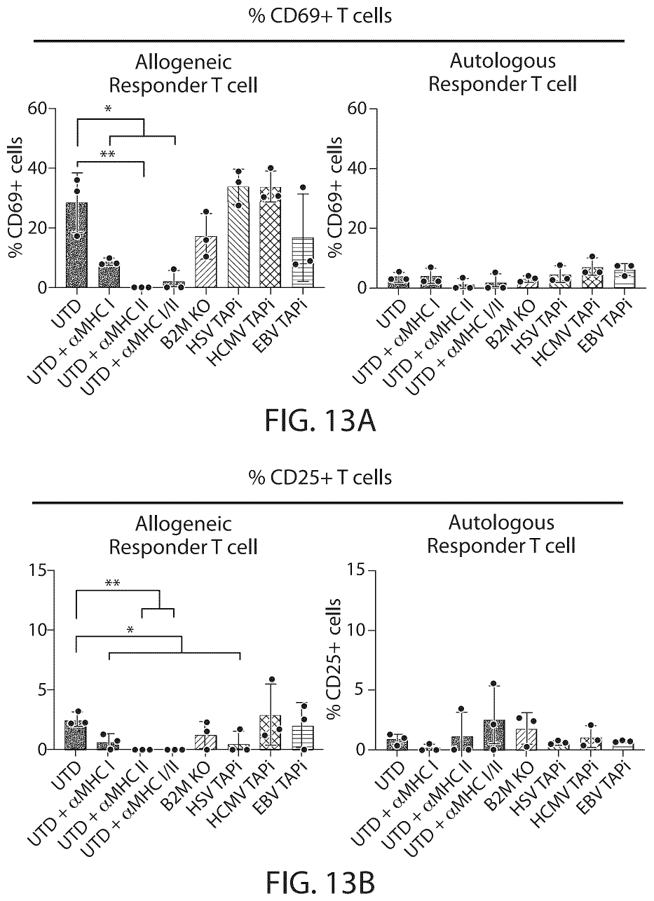

Since MHC I expression inhibits targeting by NK cells³¹, the impact of MHC I downregulation on susceptibility to NK cell killing was investigated. Like previous reports^{26,27}, 02M KO T cells were susceptible to autologous NK cell lysis and induced NK cell degranulation, as measured by CD107a expression. Compared to β2M KO T cells, T cells expressing EBV viral TAPi triggered significantly reduced NK cell lysis or degranulation (FIG. 6D). Similarly, MHC I expression mediates allogeneic T cell responses due to a mismatch between the MHC and TCR. To measure the allogeneic response of TAPi-expressing T cells, a mixed lymphocyte reaction (MLR) was performed. Transduced T cells were incubated with autologous or allogeneic labeled responder T cells in the presence or absence of MHC I and II blocking antibodies. Responder T cell activation was measured by proliferation (FIG. 6E) and changes in CD69 and CD25 expression (FIGS. 13A-13B). T cells transduced with viral TAPi, especially EBV BNLF2a, induced less allogeneic responder T cell activation, comparable to MHC I and/or MHC II blockade.

Next, the ability of TAPi-expressing T cells to present cytoplasmic antigens was tested by assessing the presentation of a peptide derived from the highly immunogenic HCMV pp65 protein. The immunogenic NLV peptide is presented on the HLA-A*02:01 allele and drives NLV-specific CD8⁺ T cells to secrete IFNγ.³² Lines of NLV-specific “responder T cells” were first generated by serial stimulation of PBMC derived from HLA-A*02:01 healthy donors who had evidence of CMV-specific memory responses. A panel of “stimulator T cells” derived from the same healthy donors were then generated, which were untransduced (UTD) or transduced with the constructs as shown (FIG. 6A), including the three different viral TAPi. Co-cultures of “stimulator cells” with “responder cells” demonstrated that viral TAPi expression, especially when derived from HSV or EBV, reduced antigen presentation, based on a reduction of IFNγ secretion in “responder T cells” (FIG. 1D). Despite reduced antigen presentation, using a viral protein to knock down the MHC I raises the possibility of an immune response to its sequence. To measure the immunogenicity of viral TAPi transduction in T cells, normal donors with pre-existing cellular immunity to the respective TAPi viruses were identified. PBMCs from normal donors were screened with peptides known to be immunogenic and originating from HCMV, EBV, or HSV in an IFNγ ELISpot assay.³³⁻³⁶ T cells from normal donors with a detectable cellular response those viruses were then transduced with viral TAPi from the same virus and incubated with autologous CD8⁺ T cells. CD8 T cell activation was measured by IFNγ ELISpot (FIG. 1E). While T cells from an HCMV-responsive donor were activated in response to transduction with HCMV pp65, they did not respond to transduction with the CMV TAPi. Similarly, HSV- and EBV-responsive donors did not produce IFNγ in response to HSV or EBV TAPi, indicating that these viral TAP inhibitors do not elicit T cell responses, despite the donors being responsive to other known immunogenic sequences from the same viruses.

Expression of shRNA Targeting CIITA Decreases Cell Surface Levels of MHC Class II

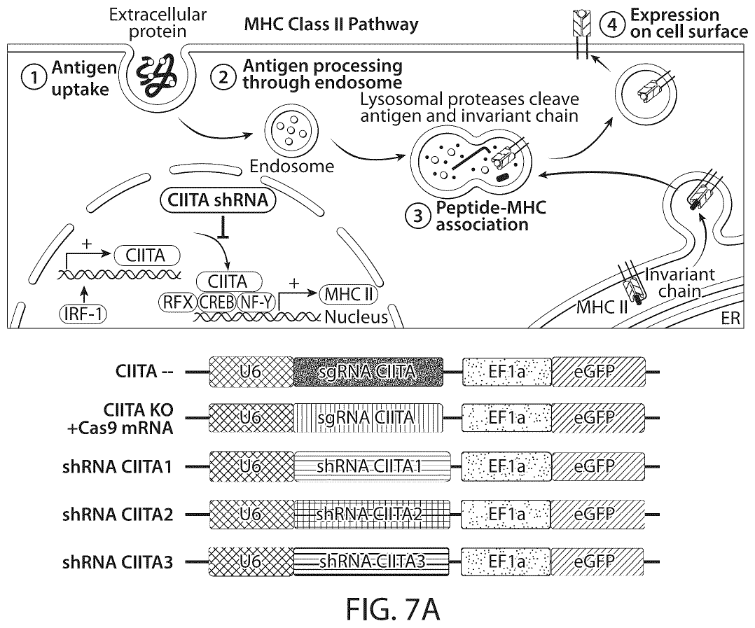

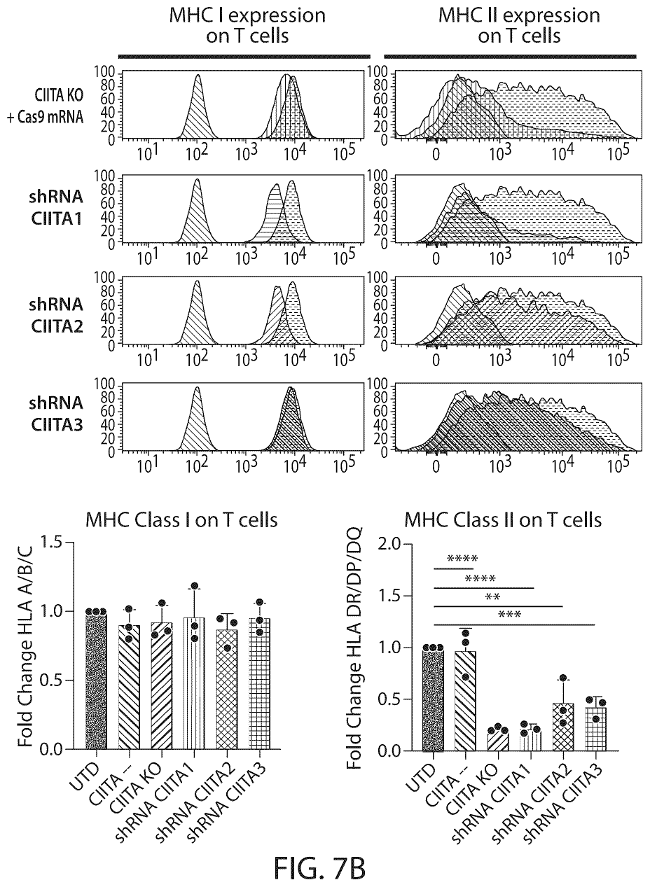

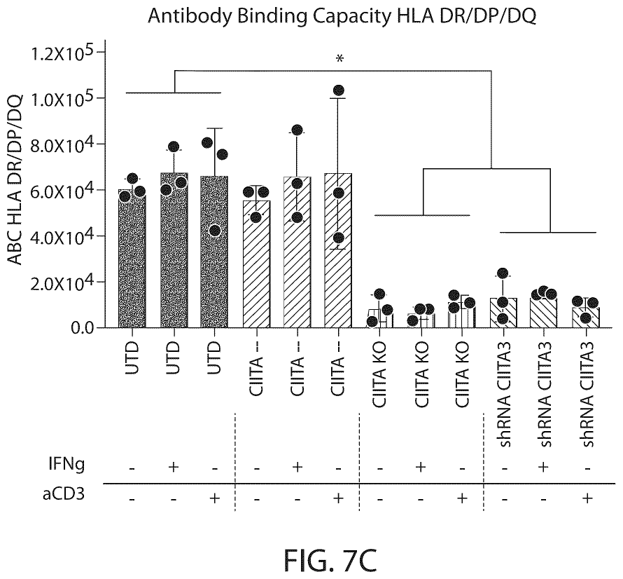

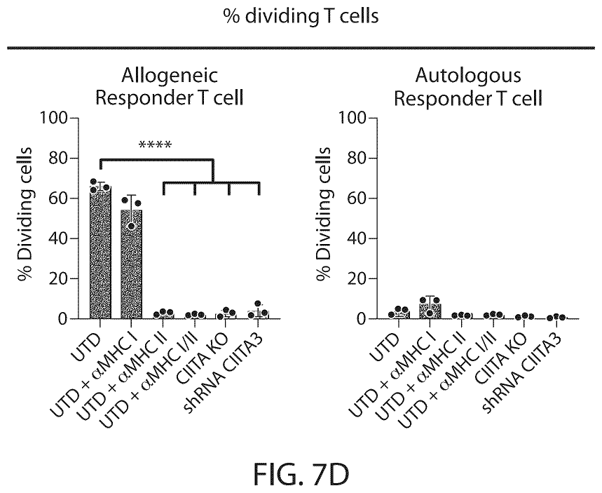

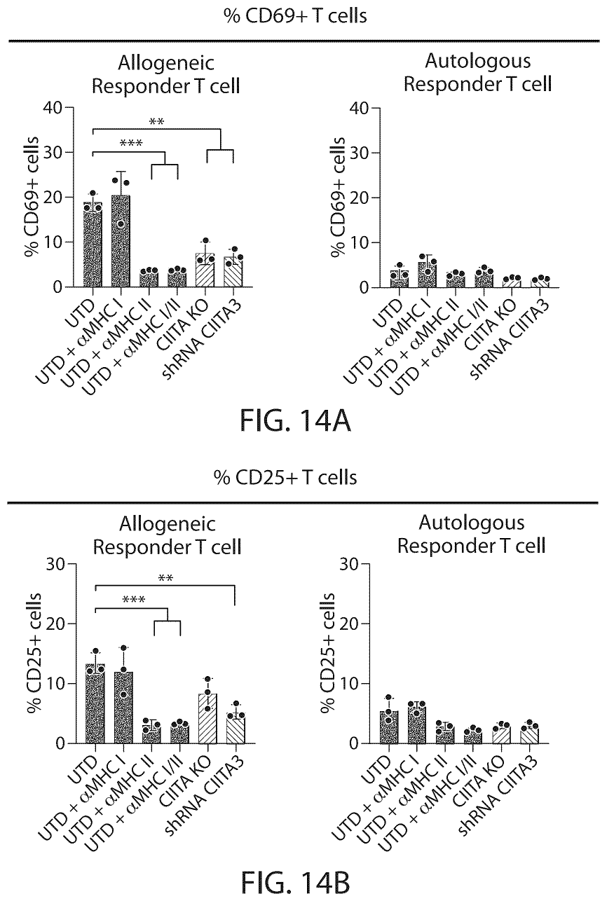

Activated human T cells express high levels of MHC class II molecules. In gene-modified cells, high MCH II could trigger rejection via antigen cross-presentation of the genetic modifications.^{3,37} Similar to MHC class I, direct targeting of MHC class II expression with DNA-editing techniques is highly complex and potentially patient-specific, as these genes are highly polymorphic and harbor significant allelic variation.³⁸ MHC Class II expression reduction was tested by targeting CIITA, the main regulatory factor that controls the transcription of MHC II genes.³⁹ To avoid the use of gene-editing and double-strand breaks, an shRNA targeting CIITA was encoded into the lentiviral vectors (FIG. 7A) using a panel of shRNA sequences. The shRNA vectors were also compared to gene knockout of CIITA with CRISPR/Cas9. It was noted that primary human T cells transduced with CIITA-targeting shRNA had reduced cell surface expression of MHC II, comparable to CIITA KO, without affecting MHC I expression (FIG. 7B). Both CIITA-targeting strategies, CRISPR/Cas9 and shRNA, rendered T cells with less than 20000 MHC Class II molecules on their surface, which was unaffected by additional stimulation with IFNγ or αCD3-antibody (FIG. 7C). However, only shRNA CIITA3 reduced MHC II expression without compromising T cell proliferation (FIG. 2C). In a mixed lymphocyte reaction (MLR) using allogeneic or autologous responder T cells, shRNA-mediated knockdown of CIITA reduced responder T cell proliferation (FIG. 7D, FIGS. 14A-14B).

Expression of the Viral TAP Inhibitor EBV BNLF2a and an shRNA Targeting CIITA can be Combined in Primary T Cells to Decrease Cell Surface Levels of Both MHC Class I and Class II

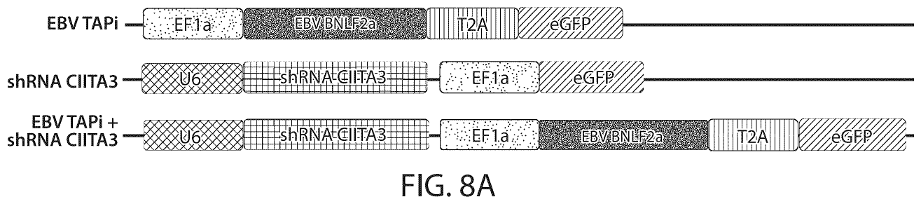

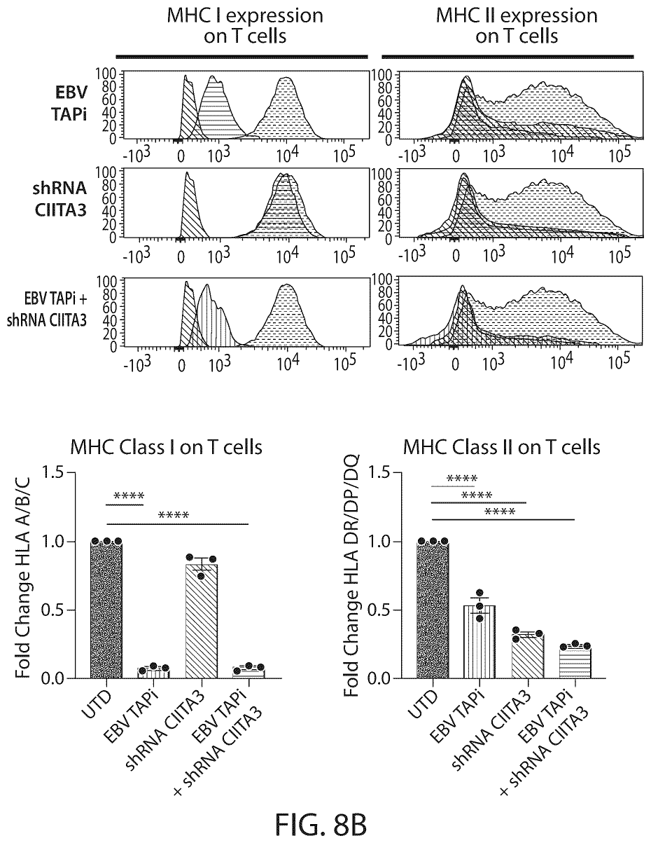

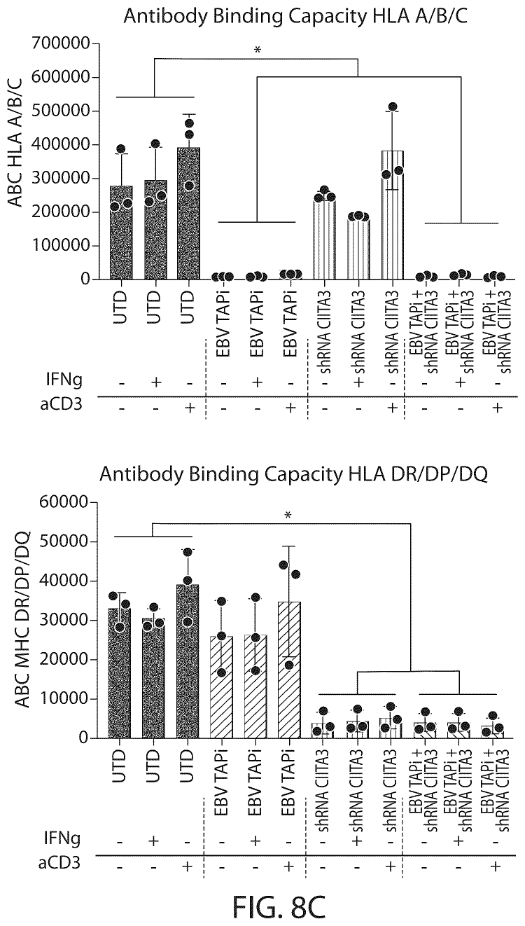

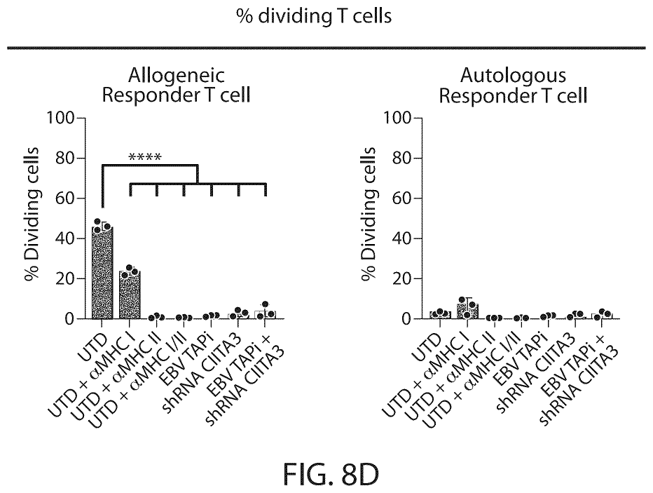

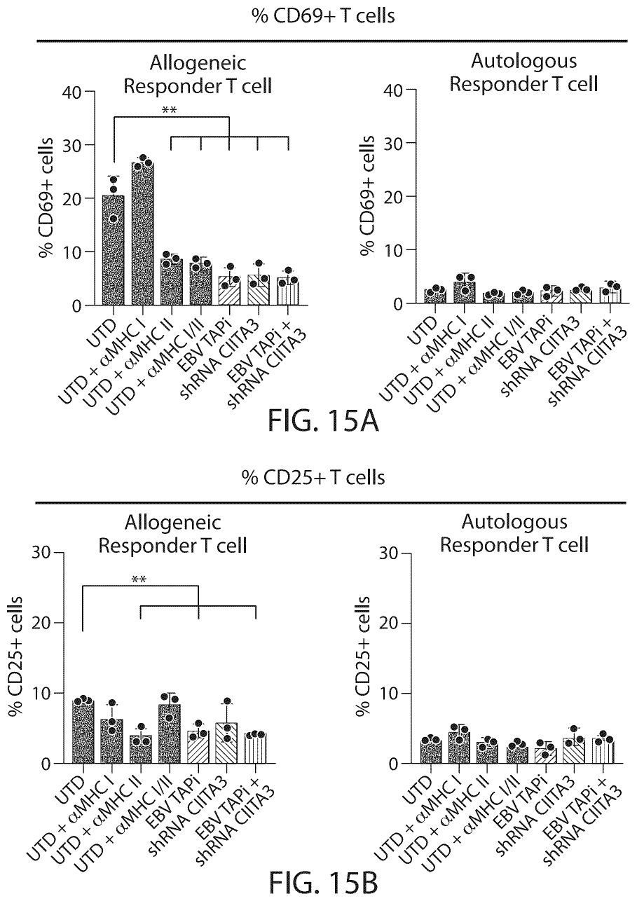

Both strategies to downregulate the cell surface expression of MHC I or II were effective separately, but the question remained as to whether these strategies could be combined. The TAPi EBV BNLF2a was selected to be combined with the shRNA CIITA3. This TAPi reduced sufficient MHC I at the cell surface to suppress antigen presentation, while the remaining MHC I at the cell surface can potentially suppress NK cell activation. These MHC I and II downregulation strategies were combined by including both EBV TAPi and shRNA CIITA3 into one lentiviral vector (FIG. 8A). When transduced into primary human T cells, the combined EBV-TAPi/shRNA-CIITA3 vector reduced MHC I and II expression (FIGS. 8B-8C) and reduced proliferative responses in MLRs (FIGS. 8D, FIGS. 15A-15B). This demonstrates that gene-modified primary T cells can successfully evade cellular immune responses by the MHC class I and II downregulation strategies presented herein, creating “stealth” T cells.

### Stealth-Enabled αCD19 CAR T Cells are Functional In Vitro and In Vivo

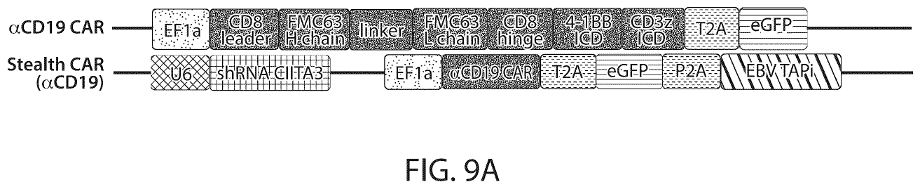

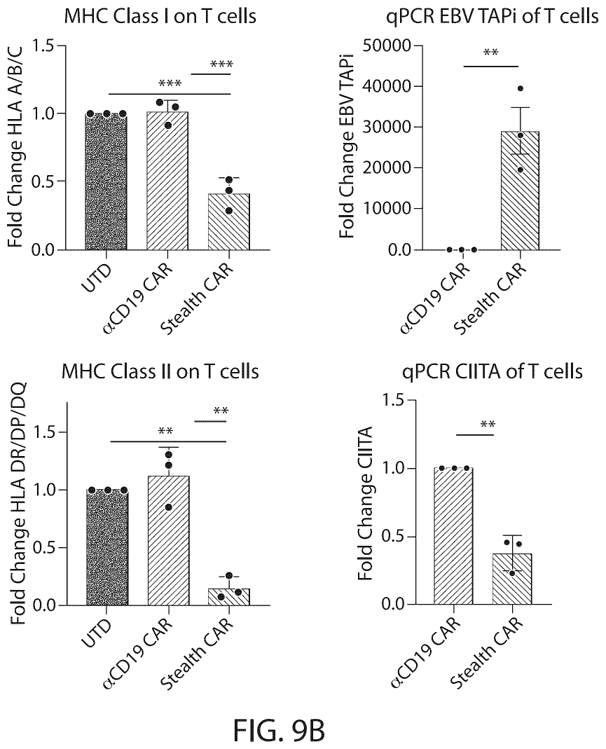

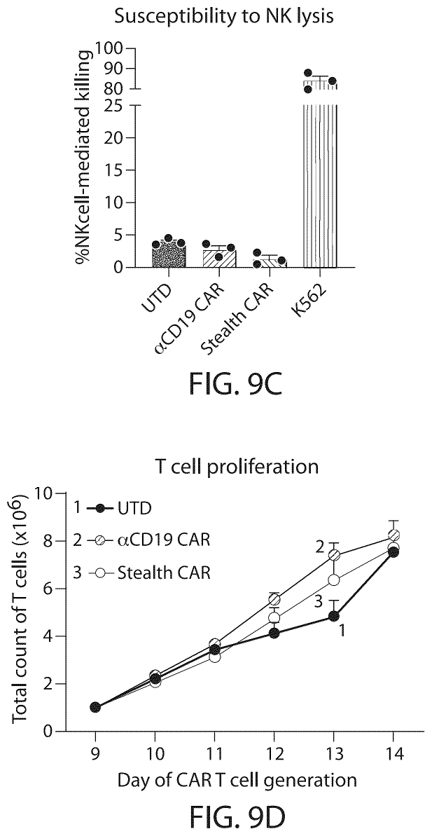

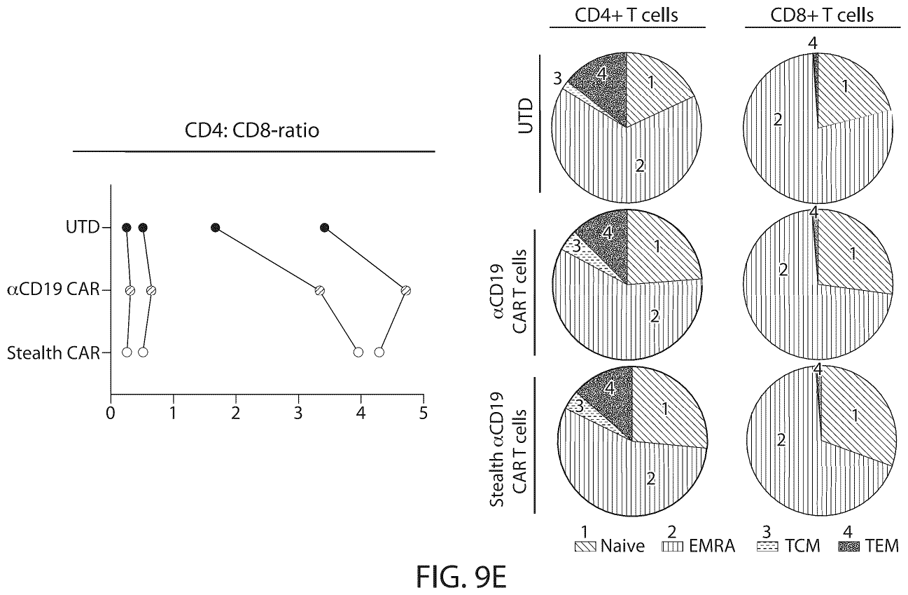

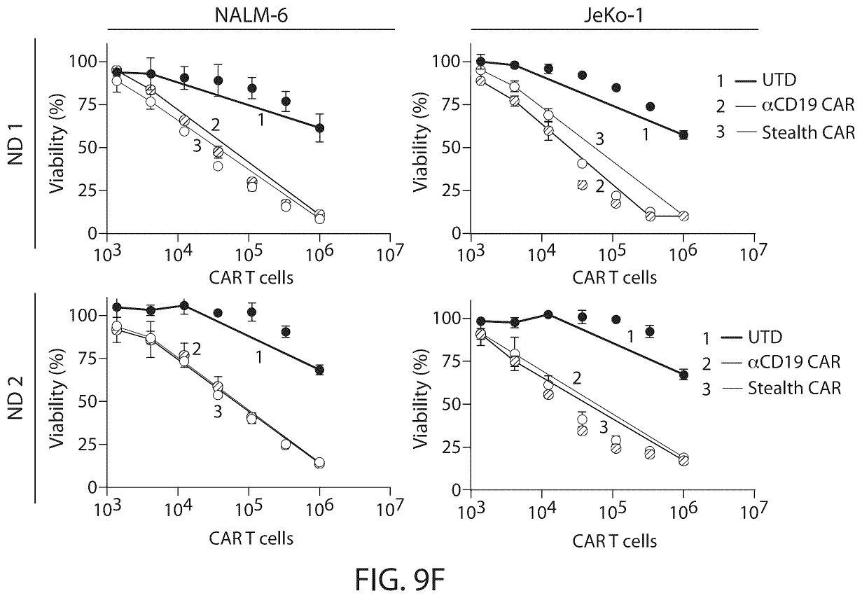

The murine scFv FMC63, which recognizes CD19 and is used in four of the six FDA-approved CAR-T cell products, has been reported to elicit autologous T cell responses in patients.^{3,6} Thus, the stealth strategy was tested in the context of FMC63 CARs to verify they retain function and avoid eliciting cellular immunity. The stealth FMC63-based αCD19 CAR was generated by incorporating both the EBV TAPi and shRNA CIITA3 (FIG. 9A). The stealth αCD19 CAR-T cells had reduced MHC I and II molecules on their cell surface compared to the T cells transduced with the αCD19 CAR alone and had robust expression of EBV TAPi and reduced CIITA mRNA expression compared to the αCD19 CAR alone by qPCR (FIG. 9B). Interestingly, this reduction of MHC I molecules at the cell surface did not increase NK cell cytotoxicity, and proliferation of the CAR-T cells was unchanged compared to the untransduced T cells (FIGS. 9C-9D). Additionally, phenotypic analysis by CD4, CD8, CCR7, and CD45RA further showed no differences in CD4/CD8 ratios and memory phenotypes comparing the αCD19 CAR-T cells with or without the stealth technology (FIG. 9E). The stealth αCD19 CAR-T cells also maintained their ability to target tumor cells in vitro. When co-incubated with luciferase-expressing acute lymphoblastic leukemia (ALL) NALM6 cells or mantle cell lymphoma JeKo-1 cells, stealth αCD19 CAR-T cells reduced tumor cell viability to the same extent as αCD19 CAR-T cells (FIG. 9F).

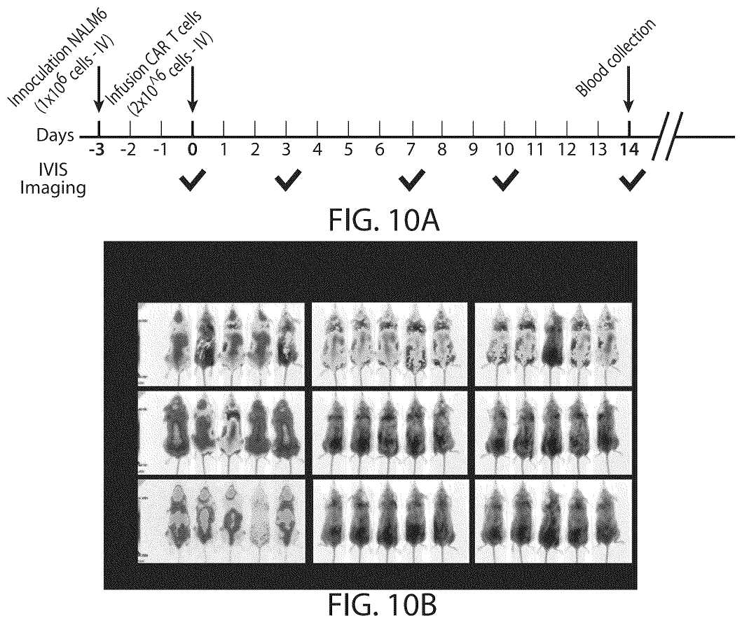

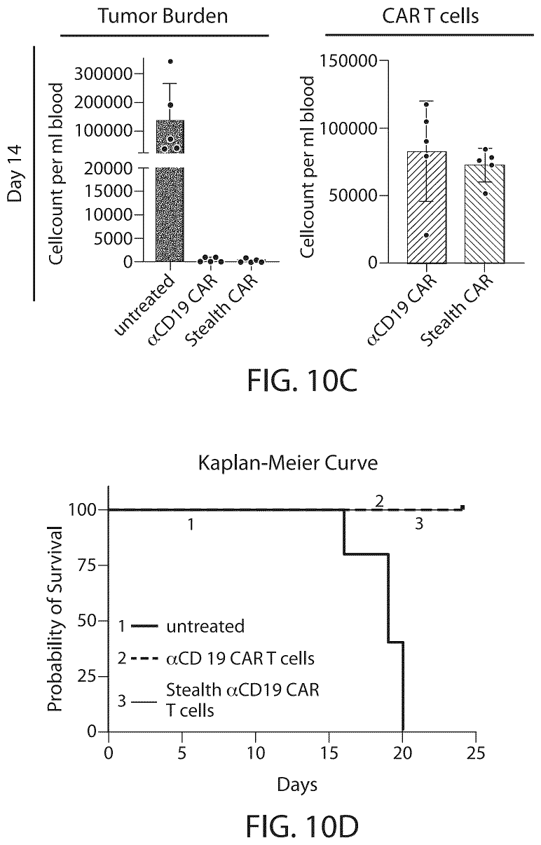

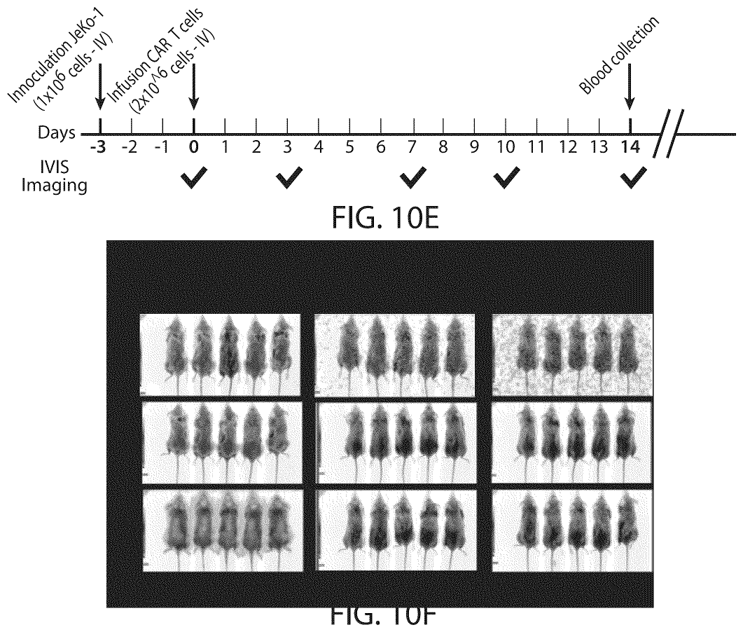

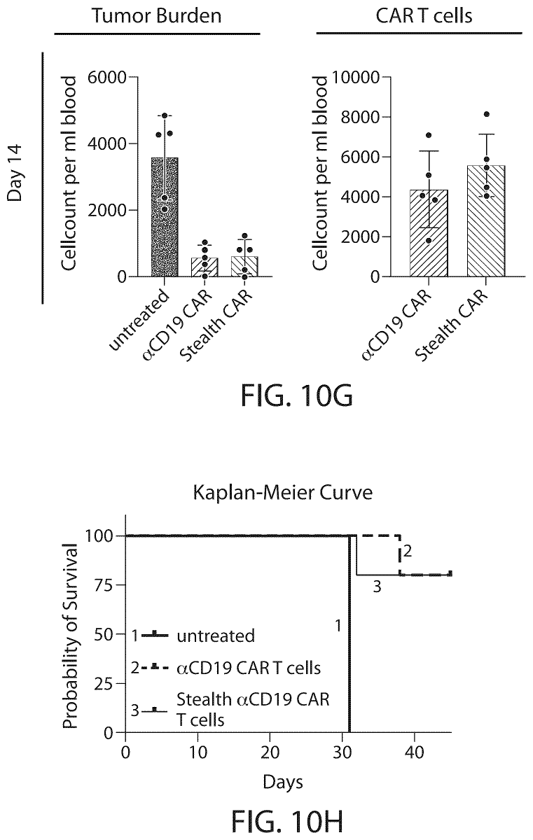

The in vivo functionality was also investigated. After tumor engraftment with NALM6 cells or JeKo-1, mice were left untreated or injected with αCD19 CAR-T cells with or without stealth technology. CAR-T cell expansion in the blood was assessed by flow cytometry, and tumor clearance was measured by bioluminescence imaging (BLI) (FIG. 10A). Mice treated with αCD19 CAR-T cells or stealth αCD19 CAR-T cells showed comparable tumor clearance, while tumors vastly expanded in untreated mice by BLI (FIGS. 10B & 10F). Both αCD19 CAR-T cells and stealth αCD19 CAR-T cells expanded similarly in the blood, as observed at day 14 by the presence of GFP+CD3+ cells (FIGS. 10C & 10G). Tumor cells (GFP+CD3− NALM6 cells) were absent or minimally present in the blood of CAR-T cell-treated mice, while a large expansion was found in the untreated group, similar to the BLI imaging. Kaplan-Meier survival curves demonstrated no difference in the survival of mice treated with αCD19 CAR-T cells with or without the additional stealth technology (FIGS. 10D & 10H). In summary, stealth αCD19 CAR-T cells retained their ability to recognize and clear CD19-expressing cells both in vitro and in vivo.

Stealth-Enabled αCD19 CAR T Cells Evade CAR-Mediated Immune Recognition by T Cells from Patients Who Received a Single or Second Infusion of αCD19 CAR T Cells.

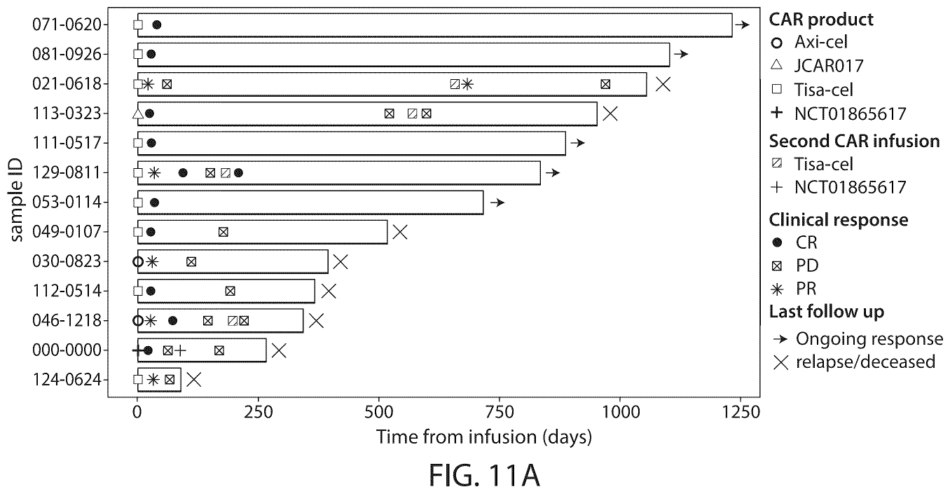

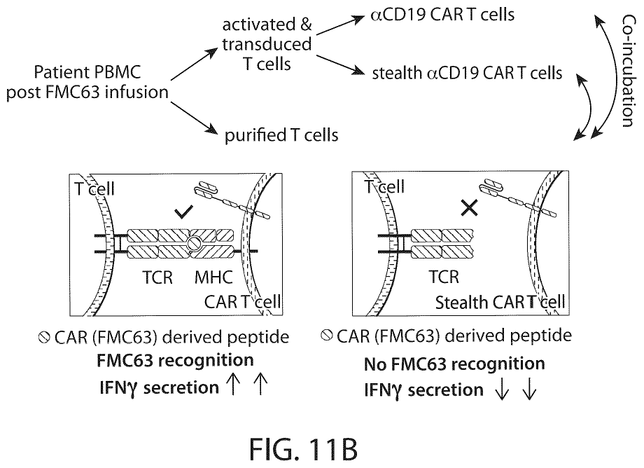

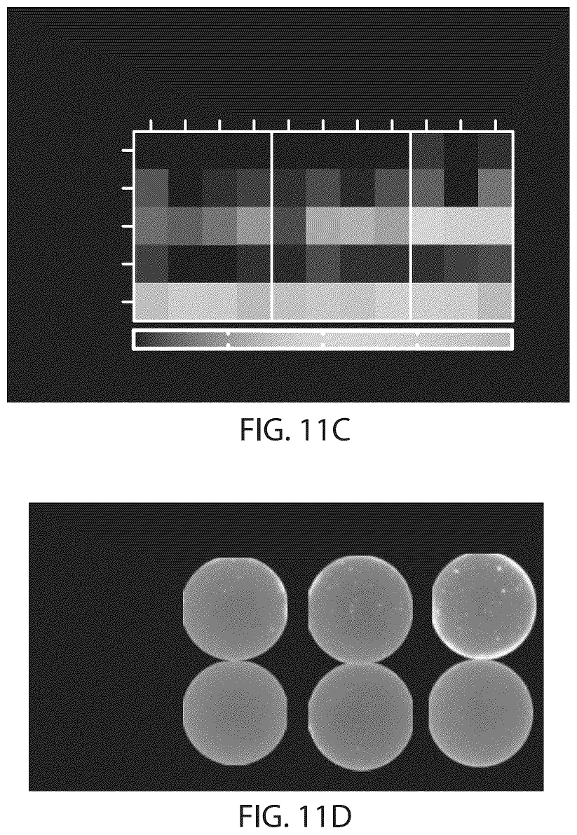

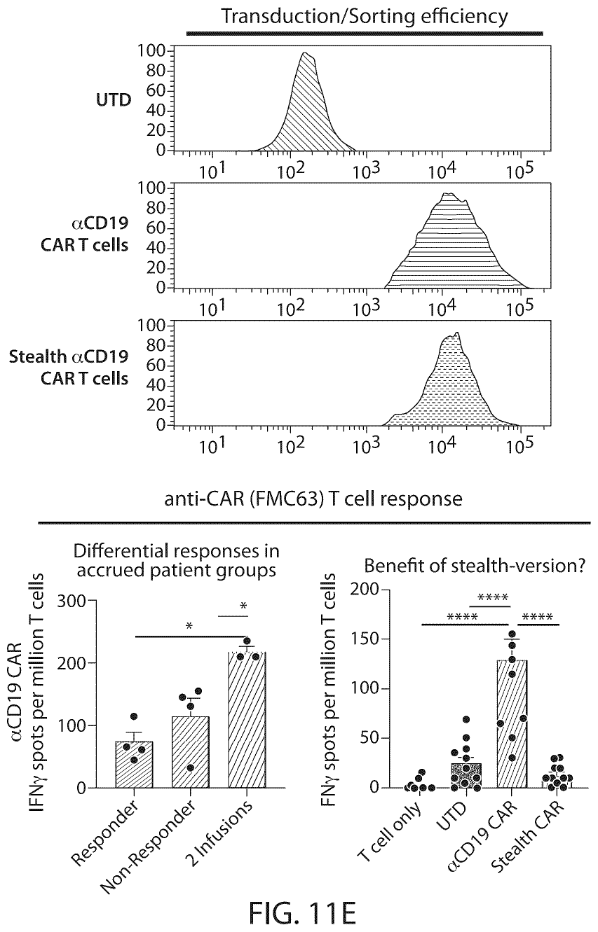

Next, the ability of the stealth technology to avoid antigen presentation of immunogenic CAR sequences was tested. 11 patients who had received autologous FMC63-based CARs either once or twice were identified. Four of these patients had initial responses of their tumor to their CAR T cell product, while four had tumors that did not respond to CAR T cells. Three patients received a second infusion of CAR T cells due to tumor progression after the first infusion (FIG. 11A). To assess whether T cells from these patients could be activated by their autologous T cells expressing the FMC63-based αCD19 CAR, fresh αCD19 CAR-T cells were made with or without stealth technology from their T cells (collected 3 months post-infusion, absent of CAR) as “stimulators” and co-cultured these with autologous, untransduced T cells as “responders” in an IFNγ ELISpot assay (FIGS. 11B-11E). Responder T cells became activated in the presence of FMC63-based αCD19 CAR-T cell products, but not UTD cells or stealth αCD19 CAR-T cells. Activation of responder T cells was particularly high in subjects who had received two infusions of FMC63-based CAR T cells and in 3 of the 4 non-responders. These data suggest that multiple infusions may increase anti-CAR immunity in patients and that a fraction of non-responders may robustly reject their autologous αCD19 CAR-T cells when reinfused. However, larger patient numbers would be required to establish a correlation between a lack of response and CAR T cell rejection.

### Stealth αCD19 CAR T Cells Reduce Allogeneic Responses In Vitro and In Vivo

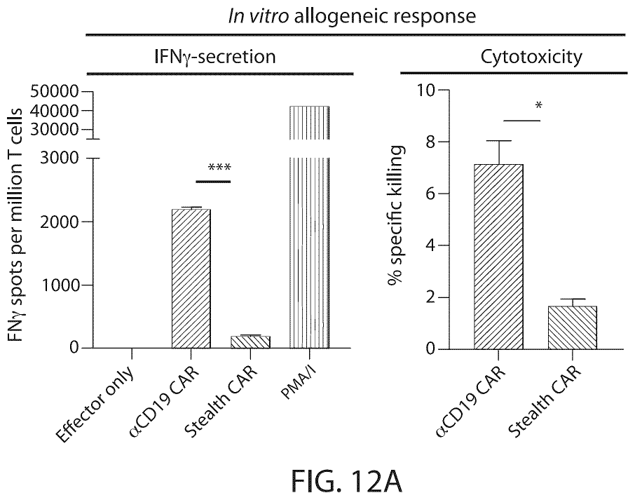

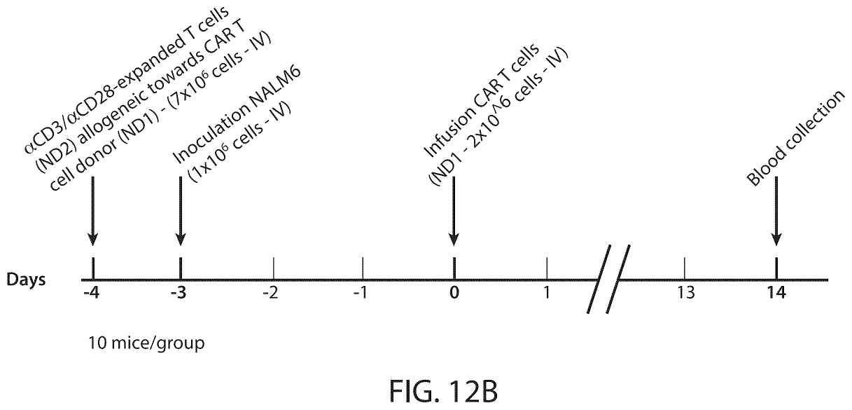

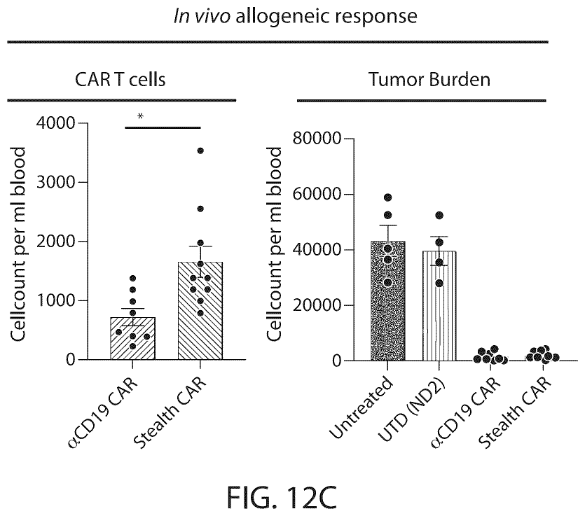

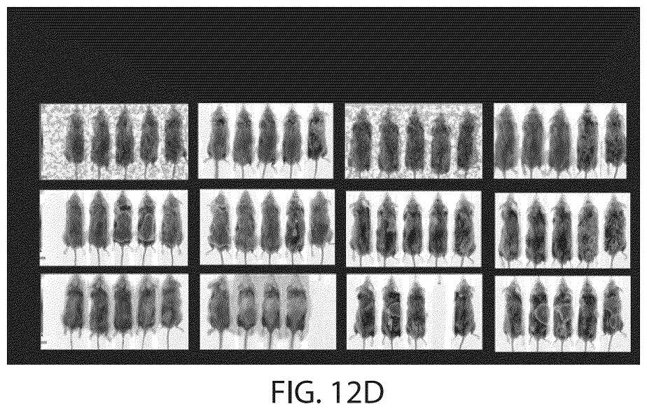

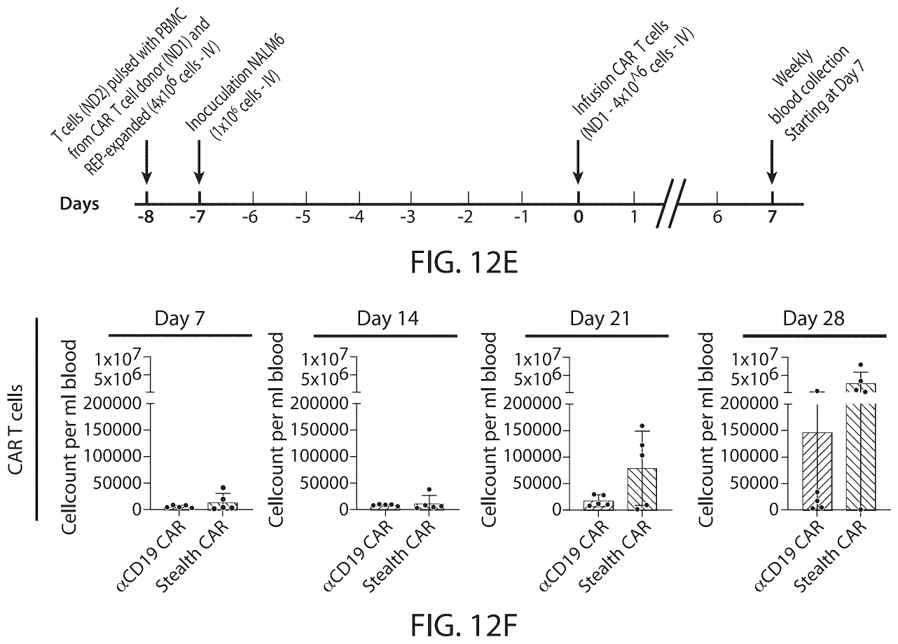

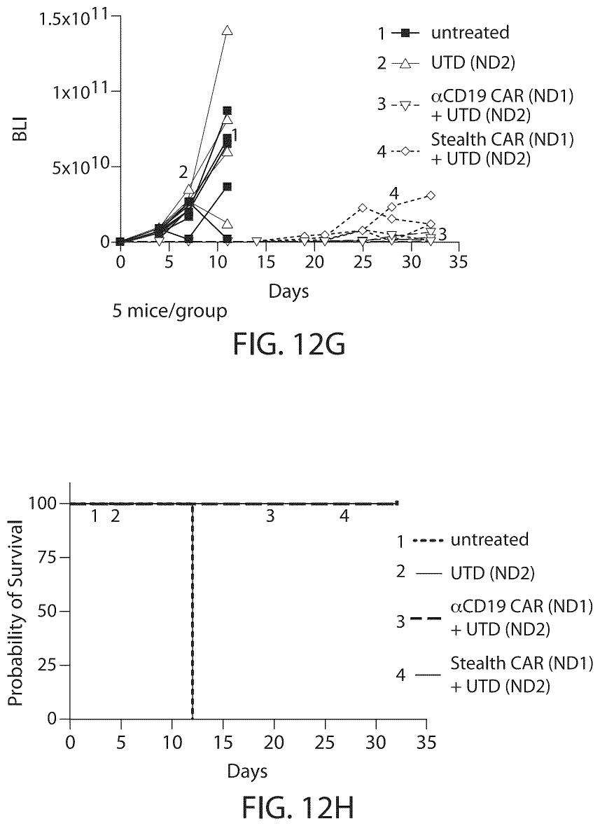

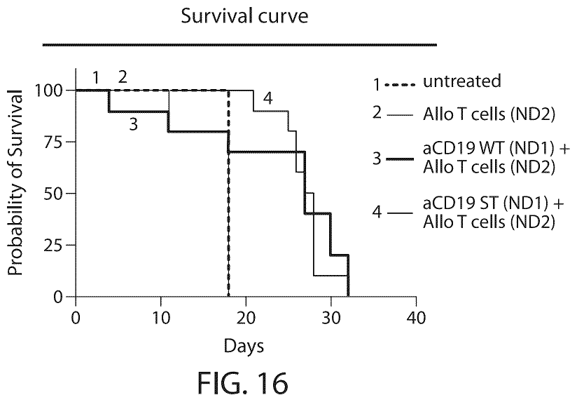

Finally, the evasion mechanism of stealth αCD19 CAR-T cells towards allogeneic T cells was investigated. When αCD19 CAR-T cells or stealth αCD19 CAR-T cells were co-incubated with expanded allogeneic T cells (expanded by αCD3/αCD28 beads) in vitro, the stealth technology reduced both IFNγ-secretion and cytotoxicity towards the CAR-T cells (FIG. 12A). A previously reported in vivo mouse model was also implemented,⁴⁰ where αCD3/αCD28-expanded allogeneic T cells were injected before NALM6 inoculation and subsequent treatment with CAR-T cells (FIG. 12B). Stealth CAR-T cells had expanded significantly more in the blood on day 14 (via flow cytometry) compared to αCD19 CAR-T cells, despite the presence of similar levels of allogeneic T cells and tumor burden (FIGS. 12C-12D). However, because the stealth system did not eliminate the large numbers of untransduced, activated T cells, the incidence and severity of xenogeneic GvHD (as indicated by fur loss and sclerosis) was early and high, resulting in no change in survival (FIG. 16). Therefore, a second allogeneic model was implemented using allogeneic T cells that were primed by pulsing twice with irradiated PBMC originating from the CAR T-cell donor to boost the allogeneic response. These primed cells were then expanded according to the rapid expansion protocol²⁸ before injecting into the mice (FIG. 12E). In this model, the stealth αCD19 CAR-T cells robustly expanded over course of 4 weeks compared to αCD19 CAR-T cells (FIG. 12F) and had comparable anti-tumor activity (FIGS. 12G-12H). The primed allogeneic T cells allowed for longer monitoring of the mice before the onset of severe xenogeneic GvHD. Importantly, the stealth αCD19 CAR-T cells expanded more robustly than the αCD19 CAR T cells.

### Discussion

CAR-T cells targeting CD19 have provided frequent, complete responses in patients with hematological disorders deemed previously uncurable. Albeit, certain hurdles in both autologous and allogeneic settings remain.^{15,16} Current clinical trials with allogeneic CAR T cells employ a means to reduce MHC I and/or class II cell surface presentation to evade one of these hurdles, the host immune response.^{3,4} It is shown herein that the inclusion of stealth transgenes, EBV TAPi BNLF2a and shRNA targeting CIITA, effectively reduced MHC cell surface molecules to evade autologous and allogeneic T cell responses. These stealth transgenes were incorporated within the CAR-transduction vector to develop a one-shot transduction to produce CAR-T cells with T cell-evasive properties. This simplified approach is particularly valuable as it does not rely on CRISPR/Cas9 gene-editing technique to ablate MHC I/II from the cell surface.^{4,41} CRISPR/Cas9 can introduce off-target effects through INDELs that promote aberrant mRNA or protein products, which are increased upon introducing multiple targets.^{20,42} Since CRISPR/Cas9 is also being investigated for a variety of other targets in CAR-T cells, such as targets to increase CAR T cell fitness and persistence,^{22,41} alternate solutions to reduce HLA from the CAR-T cell surface would enable CRISPR/Cas9 to still be used for these purposes.

Evasion of T cell immunity can be especially valuable in αCD19 CAR T cell therapy, which efficiently eliminates normal B cells in addition to the intended tumor cells, thereby naturally limiting the humoral immune response to non-self CAR components. Since anti-CAR or donor-specific antibodies and their potential interference with αCD19 CAR T cell therapy is very limited,^{3,42} equipping αCD19 CAR T cells or αCD19 NK cells with a mechanism to prevent T cell immunity could have a major impact. Clinical trials with autologous CAR-T cells have shown that patients treated with CAR-T cells develop a CAR-reactive T cell response.^{3,6,11} It is demonstrated herein that the stealth CAR-T cells evade anti-CAR responses originating from the FMC63-based αCD19 CAR and obtained increased proliferation in an allogeneic model. Furthermore, an increased CAR-reactive T cell response was found in patients who received multiple FMC63-based αCD19 CAR-T cell infusions.

We did not perform an exhaustive comparison of all the ways that can be used to evade immunogenicity. Indeed, CRISPR/Cas9 and TALEN gene knockouts are frequently employed to eliminate the T cell receptor and/or B2M in allogeneic T cell products. It may also be possible to use shRNA to B2M⁴³, or base-editing technologies to mutate B2M⁴⁴. An advantage of the present disclosure is that it could be easily combined with other gene-editing strategies, such as CRISPR/Cas9, while economizing on the number of double-strand breaks or possible translocation events. Furthermore, incorporation of stealth transgenes into autologous, “simple” lentiviral-transduced autologous products could be implemented quickly, without the need to develop exhaustive sequencing-based strategies to measure off-target gene editing effects or additional release assays

Besides the potential of the stealth transgenes in CAR-T cell therapy, this stealth technology may be useful in additional settings that employ gene-modified cells, where either the transgene, junctional sequences, or the cell types are not autologous and where avoidance of early rejection can enhance the desired therapeutic effects.^{3,8,45}

### Methods

### Mice and Cell Lines

NSG mice were purchased from Jackson Laboratory and bred under pathogen-free conditions at the Center for Comparative Medicine at MGH. Experiments were performed according to protocols approved by the Massachusetts General Hospital Institutional Animal Care and Use Committee. HEKT cells, NALM-6 (ALL), JeKo-1 (MCL), and K562 (CML) were purchased from the American Type Culture Collection, maintained under conditions as outlined by the supplier and, where indicated, transduced to express click beetle green luciferase and enhanced GFP. Cell lines were periodically authenticated by STR profiling, and routinely tested to exclude mycoplasma infection.

### (Stealth) CAR T Cell Production

Human T cells were purified (Stem Cell Technologies) from healthy donor leukapheresis products purchased from the MGH blood bank under an Institutional Review Board-exempt protocol. T cells from patients treated with axicabtagene ciloleucel or tisagenlecleucel at MGH were collected on an IRB-approved protocol (16-206) with written informed consent; PMBC from one subject treated at Seattle Cancer Care Alliance with two infusions of autologous FMC63-based CAR T cells were provided by Dr. Turtle and collected with written informed consent. In brief, bulk human T cells were activated on day 0 using CD3/CD28 Dynabeads (Life Technologies) and cultured in RPMI 1640 medium with GlutaMAX and HEPES supplemented with 10% FBS and 20 IU/ml recombinant human IL-2. Lentiviral transduction was performed on day 1, and on day 5 CD3/CD28 Dynabeads were removed. Where applicable, the T cells were electroporated with Cas9 mRNA on day 5. In cases of flow-based sorting, the T cells were sorted on day 8 using the eGFP marker and expanded until day 14 to be subsequently cryopreserved. When unsorted CAR T cells were used, CAR-T cells were normalized for transduction efficiency using untransduced activated T cells from the same donor and expansion.

### Cytotoxicity Assay

To assess the cytotoxicity of CAR T cells towards target cells, CAR T cells were incubated with luciferase-expressing tumor targets at indicated E/T ratios for 24h. Remaining luciferase activity was subsequently measured with a Synergy Neo2 luminescence microplate reader (Biotek). To assess the cytotoxicity of NK cells towards stealth or CAR T cells, NK cells were purified from blood or frozen PBMCs (Stem Cell Technologies) and primed with 20 IU/ml recombinant human IL-2 before co-incubation with their respective target cells stained with CFSE (Life Technologies). After 3h of co-incubation αCD107a antibodies were added and the assay was left to incubate for another hour. After a total of 4h, cells were centrifuged, resuspended with dead/alive marker SYTOXred (Life Technologies), and assessed by flow cytometer for target cell viability and NK cell degranulation.

### ELISpot Assay

Plates with Immobilon-P membrane (Millipore) were activated with 35% Ethanol for 30 seconds, washed with PBS and incubated overnight with PBS containing anti-human IFNγ antibody (Clone NIB42, Biolegend). The next day, the plate was blocked with PBS containing 1% BSA and 5×10⁵ PBMCs or 2×10⁵ T cells were co-incubated with respective peptides, antigens, or stimulants. After 24 h, the plate was washed with PBS containing 0.05% Tween-20 and incubated overnight with PBS containing biotinylated anti-human IFNγ antibody (Clone 4S.B3, Biolegend) as detection antibody. After washing with PBS containing 0.05% Tween-20, the plate is incubated for 2 h with avidin-HRP (Biolegend), developed using the BD Elispot AEC Substrate set and analyzed with ImmunoSpot Reader systems. All antibodies were used according to the manufacturers' recommendation.

### ELISA

Interferon γ from supernatants was measured following an overnight co-incubation of NLV responder T cells with target at a E:T ratio of 1:5 using Human DuoSet ELISA kits (R&D systems).

### Flow Cytometry

Generally, cells were stained in the dark for 30 min at 4° C. and washed twice with RPMI before analysis. SYTOXRed or SYTOXBlue (Life Technologies) were added as dead/alive marker, and singlet discrimination was performed on both the FSC and SSC detectors. The following antibodies targeting their respective antigens were used according to the manufacturers' recommendations in combination with their respective isotype control: CD4 (SK3, Biolegend), CD8 (SKi, Biolegend), CD3 (OKT3, Biolegend), CD25 (BC96, Biolegend), CD69 (FN50, Biolegend), HLA-A/B/C (W6/32, Biolegend), HLA-DR/DP/DQ (T039, Biolegend), CD107a (H4A3, Biolegend), murine erythroid cells (TER-119, Biolegend), murine Ly6G/6C (RB6-8C5, Biolegend), murine CD11b (M1/70, Biolegend) and murine NK1.1 (PK136, Biolegend). When specified, antibody binding capacity was measured utilizing Quantum Simply Cellar beads (Bangs laboratories). Analysis was performed by FlowJo software (BD Biosciences).

### Mixed Lymphocyte Reaction (MLR) Assay

Stealth or CAR T cells were stained with CFSE (Life Technologies), whilst autologous or allogeneic T cells were stained with CellTrace Violet (Life Technologies) before being co-incubated at a 4:1 ratio in the presence of 20 IU/ml recombinant human IL-2 and either isotype or MHC I (W6/32, Biolegend) or MHC II (T039, Biolegend) or both MHC I and II blocking antibodies. Fresh IL-2 was added every other day and the T cells were pulsed with new stealth T cells and blocking antibodies on day 7 and 14. On day 16, T responder cells were stained with SYTOXRed (viability) and assessed by FCM for cell division. Allogeneicity of cells were assessed by PCR (American Red Cross) and a minimum of 5 out of 6 mismatched (HLA-A/B/C/DP/DQ/DR) were selected.

### In Vivo Study

Luciferized NALM-6 or JeKo-1 cells were harvested, washed with PBS, and counted before injecting these tumor cells (1×10⁶ NALM-6 or JeKo-1 cells per mouse) in NSG mice by tail vein. Tumor growth was confirmed 3 days later by bioluminescence, at which time the mice were treated with an injection of 2×10⁶ CAR T cells in the tail vein. Tumor progression was then longitudinally evaluated by bioluminescence emission using an Ami HT optical imaging system (Spectral Instruments) following intraperitoneal substrate injection. At day 14 (or as indicated), the blood of the mice was collected by cheek punch and analyzed by FCM for the presence of NALM-6 and CAR T cells per microliter of blood. For the allogeneic T cell mouse model, “activated” allogeneic T cells were activated with CD3/CD28 beads and mice were treated 7×10⁶ T cells per mouse. “Primed” allogeneic T cells were pulsed twice with irradiated (100 Gy) PBMC originating from the CAR T-cell donor and then expanded by a rapid expansion protocol²⁸. Mice were treated with 4×10⁶ T cells per mouse. The allogeneic T cells were injected in NSG mice by tail vein one day prior to NALM-6 tumor cell injection.

### Stealth CAR Design

DNA constructs were synthesized and cloned into a second-generation lentiviral backbone under the regulation of a human EF-1α promoter for protein translation and/or a human U6 promoter for RNA transcription. The sequences for EBV BNLF2a, HSV ICP47 and HCMV US6TAPi were synthesized and combined with eGFP by means of an 2A self-cleaving peptide. The shRNA targeting CIITA were designed with software of Dharmacon and the Whitehead institute and combined in a plasmid expressing eGFP by EF-1α promoter. Similarly, vectors with CRISPR/Cas9 guides for β2M and CIITA and eGFP expression were constructed. The lentiviral vector expressing the combination of shRNA CIITA3, EBV BNLF2a and eGFP was also constructed. For CAR constructions, plasmid expressing the FMC63-based anti-CD19 CAR were constructed in combination with expression of EBV BNLF2a and shRNA targeting CIITA.

### Statistical Methods

All statistical analyses were performed with GraphPad Prism 9 software. Data were presented as means±SEM with statistically significant differences determined by tests as indicated in figure legends.

### References in Example 2

1. Bishop M R, Dickinson M, Purtill D, et al. Second-Line Tisagenlecleucel or Standard Care in Aggressive B-Cell Lymphoma. N Engl J Med. 2021.
2. Locke F L, Miklos D B, Jacobson C A, et al. Axicabtagene Ciloleucel as Second-Line Therapy for Large B-Cell Lymphoma. N Engl J Med. 2021.
3. Wagner D L, Fritsche E, Pulsipher M A, et al. Immunogenicity of CAR T cells in cancer therapy. Nat Rev Clin Oncol. 2021; 18(6):379-393.
4. Depil S, Duchateau P, Grupp S A, Mufti G, Poirot L. ‘Off-the-shelf’ allogeneic CAR T cells: development and challenges. Nat Rev Drug Discov. 2020; 19(3):185-199.
5. Young R M, Engel N W, Uslu U, Wellhausen N, June C H. Next-Generation CAR T-cell Therapies. Cancer Discov. 2022:OF1-OF14.
6. Turtle C J, Hanafi L A, Berger C, et al. CD19 CAR-T cells of defined CD4+:CD8+ composition in adult B cell ALL patients. J Clin Invest. 2016; 126(6):2123-2138.
7. Jensen M C, Popplewell L, Cooper L J, et al. Antitransgene rejection responses contribute to attenuated persistence of adoptively transferred CD20/CD19-specific chimeric antigen receptor redirected T cells in humans. Biol Blood Marrow Transplant. 2010; 16(9):1245-1256.
8. Lamers C H, Willemsen R, van Elzakker P, et al. Immune responses to transgene and retroviral vector in patients treated with ex vivo-engineered T cells. Blood. 2011; 117(1):72-82.
9. Shah N N, Lee D W, Yates B, et al. Long-Term Follow-Up of CD19-CAR T-Cell Therapy in Children and Young Adults With B-ALL. J Clin Oncol. 2021; 39(15):1650-1659.
10. Xu X, Sun Q, Liang X, et al. Mechanisms of Relapse After CD19 CAR T-Cell Therapy for Acute Lymphoblastic Leukemia and Its Prevention and Treatment Strategies. Front Immunol. 2019; 10:2664.
11. Gauthier J, Bezerra E D, Hirayama A V, et al. Factors associated with outcomes after a second CD19-targeted CAR T-cell infusion for refractory B-cell malignancies. Blood. 2021; 137(3):323-335.
12. Nie Y, Lu W, Chen D, et al. Mechanisms underlying CD19-positive ALL relapse after anti-CD19 CAR T cell therapy and associated strategies. Biomark Res. 2020; 8:18.
13. Li X, Liu M J, Mou N, et al. Efficacy and safety of humanized CD19 CAR-T as a salvage therapy for recurrent CNSL of B-ALL following murine CD19 CAR-T cell therapy. Oncol Lett. 2021; 22(5):788.
14. Caldwell K J, Gottschalk S, Talleur A C. Allogeneic CAR Cell Therapy-More Than a Pipe Dream. Front Immunol. 2020; 11:618427.
15. Maude S L, Laetsch T W, Buechner J, et al. Tisagenlecleucel in Children and Young Adults with B-Cell Lymphoblastic Leukemia. N Engl J Med. 2018; 378(5):439-448.
16. Neelapu S S, Locke F L, Bartlett N L, et al. Axicabtagene Ciloleucel CAR T-Cell Therapy in Refractory Large B-Cell Lymphoma. N Engl J Med. 2017; 377(26):2531-2544.
17. Porter D L, Hwang W T, Frey N V, et al. Chimeric antigen receptor T cells persist and induce sustained remissions in relapsed refractory chronic lymphocytic leukemia. Sci Transl Med. 2015; 7(303):303ra139.
18. Ghorashian S, Kramer A M, Onuoha S, et al. Enhanced CAR T cell expansion and prolonged persistence in pediatric patients with ALL treated with a low-affinity CD19 CAR. Nat Med. 2019; 25(9):1408-1414.
19. Lowe K L, Mackall C L, Norry E, Amado R, Jakobsen B K, Binder G. Fludarabine and neurotoxicity in engineered T-cell therapy. Gene Ther. 2018; 25(3):176-191.
20. Tuladhar R, Yeu Y, Tyler Piazza J, et al. CRISPR-Cas9-based mutagenesis frequently provokes on-target mRNA misregulation. Nat Commun. 2019; 10(1):4056.
21. Allogene. Allogene Therapeutics Reports FDA Clinical Hold; 2021.
22. Dimitri A, Herbst F, Fraietta J A. Engineering the next-generation of CAR T-cells with CRISPR-Cas9 gene editing. Mol Cancer. 2022; 21(1):78.
23. Rezalotfi A, Fritz L, Forster R, Bosnjak B. Challenges of CRISPR-Based Gene Editing in Primary T Cells. Int J Mol Sci. 2022; 23(3).
24. Choi B D, Yu X, Castano A P, et al. CRISPR-Cas9 disruption of PD-1 enhances activity of universal EGFRvIII CAR T cells in a preclinical model of human glioblastoma. J Immunother Cancer. 2019; 7(1):304.
25. Biosciences P. 2021.
26. Kagoya Y, Guo T, Yeung B, et al. Genetic Ablation of HLA Class I, Class II, and the T-cell Receptor Enables Allogeneic T Cells to Be Used for Adoptive T-cell Therapy. Cancer Immunol Res. 2020; 8(7):926-936.
27. Lee J, Sheen J H, Lim O, et al. Abrogation of HLA surface expression using CRISPR/Cas9 genome editing: a step toward universal T cell therapy. Sci Rep. 2020; 10(1):17753.
28. Smith C, Okern G, Rehan S, et al. Ex vivo expansion of human T cells for adoptive immunotherapy using the novel Xeno-free CTS Immune Cell Serum Replacement. Clin Transl Immunology. 2015; 4(1):e31.
29. Verweij M C, Horst D, Griffin B D, et al. Viral inhibition of the transporter associated with antigen processing (TAP): a striking example of functional convergent evolution. PLoS Pathog. 2015; 11(4):e1004743.
30. Goldsmith K, Chen W, Johnson D C, Hendricks R L. Infected cell protein (ICP)47 enhances herpes simplex virus neurovirulence by blocking the CD8+ T cell response. J Exp Med. 1998; 187(3):341-348.
31. Vivier E, Tomasello E, Baratin M, Walzer T, Ugolini S. Functions of natural killer cells. Nat Immunol. 2008; 9(5):503-510.
32. Khan N, Cobbold M, Keenan R, Moss P A. Comparative analysis of CD8+ T cell responses against human cytomegalovirus proteins pp65 and immediate early 1 shows similarities in precursor frequency, oligoclonality, and phenotype. J Infect Dis. 2002; 185(8):1025-1034.
33. Lubke M, Spalt S, Kowalewski D J, et al. Identification of HCMV-derived T cell epitopes in seropositive individuals through viral deletion models. J Exp Med. 2020; 217(3).
34. Dervillez X, Qureshi H, Chentoufi A A, et al. Asymptomatic HLA-A*02:01-restricted epitopes from herpes simplex virus glycoprotein B preferentially recall polyfunctional CD8+ T cells from seropositive asymptomatic individuals and protect HLA transgenic mice against ocular herpes. J Immunol. 2013; 191(10):5124-5138.
35. Duraiswamy J, Burrows J M, Bharadwaj M, et al. Ex vivo analysis of T-cell responses to Epstein-Barr virus-encoded oncogene latent membrane protein 1 reveals highly conserved epitope sequences in virus isolates from diverse geographic regions. J Virol. 2003; 77(13):7401-7410.
36. Sukdolak C, Tischer S, Dieks D, et al. CMV-, EBV- and ADV-specific T cell immunity: screening and monitoring of potential third-party donors to improve post-transplantation outcome. Biol Blood Marrow Transplant. 2013; 19(10):1480-1492.
37. Costantino C M, Spooner E, Ploegh H L, Hafler D A. Class II MHC self-antigen presentation in human B and T lymphocytes. PLoS One. 2012; 7(1):e29805.
38. Wieczorek M, Abualrous E T, Sticht J, et al. Major Histocompatibility Complex (MHC) Class I and MHC Class II Proteins: Conformational Plasticity in Antigen Presentation. Front Immunol. 2017; 8:292.
39. Holling T M, van der Stoep N, Quinten E, van den Elsen P J. Activated human T cells accomplish MHC class II expression through T cell-specific occupation of class II transactivator promoter III. J Immunol. 2002; 168(2):763-770.
40. Mo F, Watanabe N, McKenna M K, et al. Engineered off-the-shelf therapeutic T cells resist host immune rejection. Nat Biotechnol. 2021; 39(1):56-63.
41. Razeghian E, Nasution M K M, Rahman H S, et al. A deep insight into CRISPR/Cas9 application in CAR-T cell-based tumor immunotherapies. Stem Cell Res Ther. 2021; 12(1):428.
42. Elahi R, Heidary A H, Hadiloo K, Esmaeilzadeh A. Chimeric Antigen Receptor-Engineered Natural Killer (CAR NK) Cells in Cancer Treatment; Recent Advances and Future Prospects. Stem Cell Rev Rep. 2021; 17(6):2081-2106.
43. Ramos C A, Courtney A N, Robinson S N, et al. Allogeneic NKT Cells Expressing a CD19-Specific CAR in Patients with Relapsed or Refractory B-Cell Malignancies: An Interim Analysis. Blood. 2021; 138(23 Nov. 2021):2819.
44. Webber B R, Lonetree C L, Kluesner M G, et al. Highly efficient multiplex human T cell engineering without double-strand breaks using Cas9 base editors. Nat Commun. 2019; 10(1):5222.
45. Jan M, Scarfo I, Larson R C, et al. Reversible ON- and OFF-switch chimeric antigen receptors controlled by lenalidomide. Sci Transl Med. 2021; 13(575).

1. A cell comprising:
(i) an inhibitor of transporter associated with antigen processing (TAPi) or variant thereof; and
(ii) an oligonucleotide that is complementary to a polynucleotide encoding a MHC class II transactivator protein or variant thereof, wherein the oligonucleotide is selected from the group consisting of a RNA interference (RNAi) oligonucleotide, an antisense oligonucleotide (ASO), or a CRISPR interference (CRISPRi) oligonucleotide.

2. A cell comprising:
(i) a chimeric antigen receptor (CAR); and
(ii) an inhibitor of transporter associated with antigen processing (TAPi) or variant thereof; and/or
(iii) an oligonucleotide that is complementary to a polynucleotide encoding a MHC class II transactivator protein or variant thereof, wherein the oligonucleotide is selected from the group consisting of a RNA interference (RNAi) oligonucleotide, an antisense oligonucleotide (ASO), or a CRISPR interference (CRISPRi) oligonucleotide.

3. The cell of claim 1 or claim 2, wherein the oligonucleotide is complementary to any one of SEQ ID NOs: 7-12.

4. The cell of claim 1 or claim 2, wherein the oligonucleotide is complementary to SEQ ID NO: 7.

5. The cell of any one of claims 1-4, wherein the TAPi or variant thereof decreases expression of MHC class I.

6. The cell of any one of claims 1-5, wherein the TAPi is a viral TAPi.

7. The cell of any one of claims 1-6, wherein the TAPi is a Herpesvirus TAPi.

8. The cell of any one of claims 1-7, wherein the TAPi is selected from the group consisting of a Herpes Simplex virus (HSV) TAPi, Human Cytomegalovirus (HCMV) TAPi, or Epstein-Barr virus (EBV) TAPi.

9. The cell of any one of claims 1-8, wherein the TAPi is selected from the group consisting of a Herpes Simplex virus (HSV) ICP47 TAPi, Human Cytomegalovirus (HCMV) US6 TAPi, or Epstein-Barr virus (EBV) BNLF2a TAPi.

10. The cell of any one of claims 1-9, wherein the TAPi comprises an amino acid sequence that is at least 85% identical to any one of SEQ ID NOs: 1-3.

11. The cell of any one of claims 1-9, wherein the TAPi comprises an amino acid sequence of any one of SEQ ID NOs: 1-3.

12. The cell of any one of claims 1-11, wherein the RNAi oligonucleotide is selected from the group consisting of a siRNA, a miRNA or a shRNA.

13. The cell of claim 12, wherein the RNAi oligonucleotide is a shRNA.

14. The cell of claim 13, wherein the shRNA comprises a nucleic acid sequence of SEQ ID NO: 3.

15. The cell of any one of claims 12-14, wherein the shRNA comprises a nucleic acid sequence of SEQ ID NO: 13.

16. The cell of any one of claims 1-15, wherein the cell is a eukaryotic cell.

17. The cell of any one of claims 1-16, wherein the cell is an immune cell.

18. The cell of any one of claims 1-17, wherein the immune cell is a T cell.

19. The cell of claim 1, wherein the cell further comprises a chimeric antigen receptor (CAR).

20. The cell of claim 2-19, wherein the CAR comprises:
(i) an extracellular target binding domain;
(ii) a transmembrane domain; and
(iii) an intracellular signaling domain.

21. The cell of claim 20, wherein the extracellular target binding domain binds to any one of CD19, CD79b, TACI, BCMA, MUC1, MUC16, B7H3, mesothelin, CD70, PSMA, PSCA, EGFRvIII, claudin6, binds to any pair of CD19/CD79b, BCMA/TACI, or is a TriPRIL antigen binding domain.

22. The cell of claim 21, wherein the extracellular target binding domain binds to CD19.

23. The cell of any one of claims 20-22, wherein the extracellular target binding domain is not derived from a human polypeptide sequence.

24. The cell of claim of any one of claims 20-22, wherein the extracellular target binding domain is derived from a murine polypeptide sequence.

25. The cell of any one of claims 20-24, wherein extracellular target binding domain comprises a V H amino acid sequence that has at least 85% identify to SEQ ID NO: 39 and a V L amino acid sequence that has at least 85% identify to SEQ ID NO: 40.

26. The cell of any one of claims 20-25, wherein the transmembrane domain is selected from the group consisting of alpha chain of a T cell receptor, beta chain of a T cell receptor or zeta chain of a T cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), 4-1BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD1 Id, ITGAE, CD103, ITGAL, CD11a, LFA-1, ITGAM, CD11b, ITGAX, CD11c,ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Lyl08), SLAM (SLAMFI, CD150, IPO-3), BLAME (SLAMF8), SELPLG (CD162), LTBR, PAG/Cbp, NKp44, NKp30, NKp46, NKG2D, and/or NKG2C.

27. The cell of any one of claims 20-26, wherein the intracellular signaling domain is selected from the group consisting of CD28, 4-1BB, CD27, TCR-zeta, FcR-gamma, FcR-beta, CD3-gamma, CD3-theta, CD3-sigma, CD3-eta, CD3-epsilon, CD3-zeta, CD22, CD79a, CD79b, and CD66d.

28. The cell of any one of claims 20-27, wherein the CAR comprises an amino acid sequence having at least 85% identify to SEQ ID NO: 41 and a nucleic acid sequence having at least 85% identity to SEQ ID NO: 17 or 18.

29. A polynucleotide comprising a nucleic acid sequence encoding (i) a TAPi or variant thereof and (ii) an oligonucleotide that is complementary to a gene encoding a MHC class II transactivator protein.

30. The polynucleotide claim 29, wherein the TAPi is a viral TAPi.

31. The polynucleotide of claim 29 or claim 30, wherein the TAPi or variant thereof decreases expression of MHC class I.

32. The polynucleotide of any one of claims 29-31, wherein the TAPi is a Herpes Simplex Virus (HSV) TAPi.

33. The polynucleotide of any one of claims 29-32, wherein the TAPi is selected from the group consisting of a Herpes Simplex virus (HSV) TAPi, Human Cytomegalovirus (HCMV) TAPi, or Epstein-Barr virus (EBV) TAPi.

34. The polynucleotide of any one of claims 29-33, wherein the TAPi is selected from the group consisting of a Herpes Simplex virus (HSV) ICP47 TAPi, Human Cytomegalovirus (HCMV) US6 TAPi, or Epstein-Barr virus (EBV) BNLF2a TAPi.

35. The polynucleotide of any one of claims 29-34, wherein the TAPi comprises an amino acid sequence that is at least 85% identical to any one of SEQ ID NOs: 1-3.

36. The polynucleotide of any one of claims 29-34, wherein the TAPi comprises an amino acid sequence of any one of SEQ ID NOs: 1-3.

37. The polynucleotide of any one of claims 29-36, wherein the oligonucleotide is complementary to any one of SEQ ID NOs: 7-12 or a variant thereof.

38. The polynucleotide of any one of claims 29-37, wherein the oligonucleotide is complementary to SEQ ID NO: 7 or a variant thereof.

39. The polynucleotide of any one of claims 29-38, wherein the oligonucleotide is selected from the group consisting of a RNAi oligonucleotide or a CRISPR interference guide RNA.

40. The polynucleotide of any one of claim 39, wherein the RNAi oligonucleotide is selected from the group consisting of a siRNA, a miRNA or a shRNA.

41. The polynucleotide of claim 39 or claim 40, wherein the RNAi oligonucleotide is an shRNA.

42. The polynucleotide of claim 41, wherein the shRNA is encoded by a nucleic acid sequence comprising of SEQ ID NO: 13.

43. The polynucleotide of any one of claims 29-42 further comprising a nucleic acid sequence encoding chimeric antigen receptor (CAR).

44. The polynucleotide of claim 43, wherein the CAR comprises:
(i) an extracellular target binding domain;
(ii) a transmembrane domain; and
(iii) an intracellular signaling domain.

45. The polynucleotide of claim 44, wherein the extracellular target binding domain binds to any one of CD19, CD79b, TACI, BCMA, MUC1, MUC16, B7H3, mesothelin, CD70, PSMA, PSCA, EGFRvIII, claudin6, binds to any pair of CD19/CD79b, BCMA/TACI, or is a TriPRIL antigen binding domain.

46. The polynucleotide of claim 44 or claim 45, wherein the extracellular target binding domain binds to CD19.

47. The polynucleotide of any one of claims 44-46, wherein the extracellular target binding domain is not derived from a human polypeptide sequence.

48. The polynucleotide of any one of claims 44-47, wherein the extracellular target binding domain is derived from a murine polypeptide sequence.

49. The polynucleotide of any one of claims 44-48, wherein the transmembrane domain is selected from the group consisting of alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), 4-1BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c,ITGB1, CD29, ITGB2, CD18, LFA-1, ITGB7, TNFR2, DNAM1 (CD226), SLAMF4 (CD244, 2B4), CD84, CD96 (Tactile), CEACAMI, CRT AM, Ly9 (CD229), CD160 (BY55), PSGL1, CD100 (SEMA4D), SLAMF6 (NTB-A, Lyl08), SLAM (SLAMFI, CD150, IPO-3), BLAME (SLAMF8), SELPLG (CD162), LTBR, PAG/Cbp, NKp44, NKp30, NKp46, NKG2D, and/or NKG2C.

50. The polynucleotide of any one of claims 44-49, wherein the intracellular signaling domain is selected from the group consisting of CD28, 4-1BB, CD27, TCR-zeta, FcR-gamma, FcR-beta, CD3-gamma, CD3-theta, CD3-sigma, CD3-eta, CD3-epsilon, CD3-zeta, CD22, CD79a, CD79b, and CD66d.

51. The polynucleotide of any one of claims 44-50, comprising a nucleic acid sequence that has at least 85% identity to SEQ ID NO: 17-18.

52. The polynucleotide of any one of claims 44-50, comprising a nucleic acid sequence of SEQ ID NO: 19 and a nucleic acid sequence of SEQ ID NO: 20, 22 or 24.

53. The polynucleotide of any one of claims 29-52, wherein the polynucleotide is a vector, optionally a lentiviral vector.

54. A polynucleotide comprising an shRNA of SEQ ID NO: 13.

55. A cell comprising the polynucleotide of any one of claims 29-54.

56. A cell of any one of claims 1-27, wherein the cell comprises the polynucleotide of any one of claims 29-54.

57. A method of modifying the immunogenicity of a cell, the method comprising introducing into the cell an oligonucleotide that is complementary to a polynucleotide encoding an MHC class II complex subunit of any one of SEQ ID NOs: 7-12, wherein the oligonucleotide is selected from the group consisting of a RNA interference (RNAi) oligonucleotide, an antisense oligonucleotide (ASO), or a CRISPR interference (CRISPRi) oligonucleotide.

58. A method of decreasing an immune response of a subject to a cell therapy, the method comprising introducing into cells of the cell therapy an oligonucleotide that is complementary to a polynucleotide encoding class II MHC transactivator complex protein of any one of SEQ ID NOs: 7-12, wherein the oligonucleotide is selected from the group consisting of a RNA interference (RNAi) oligonucleotide, an antisense oligonucleotide (ASO), or a CRISPR interference (CRISPRi) oligonucleotide.

59. The method of claim 57 or claim 58, further comprising introducing into cells of the cell therapy a virus-derived inhibitor of transporter associated with antigen processing (TAPi) or variant thereof.

60. The method of any one of claims 57-59, comprising introducing into cells of the cell therapy the polynucleotide of any one of claims 29-54.

61. The method of any one of claims 57-60, wherein the cell or cells are eukaryotic cells.

62. The method of any one of claims 57-61, wherein the cell or cells are immune cells.

63. The method of claim 62, wherein the immune cell or immune cells are T cells.

64. The method of any one of claims 57-63, wherein the cells are allogenic to the subject.

65. The method of any one of claims 57-64, wherein the cell therapy is a CAR-T cell therapy.

66. The method of claim 65, wherein the CAR-T cell therapy comprises an anti-CD19 CAR-T cell.

67. The method of any one of claims 58-66, wherein the subject is a human subject.

68. The method of any one of claims 58-67, wherein the method decreases natural killer cell activation.

69. A method of treating cancer in a subject, the method comprising administering the cell of any one of claims 1-28 or 55-56 to the subject.

70. The method of claim 69, wherein the cancer is a hematological cancer.

71. The method of claim 70, wherein the hematological cancer is selected from the group consisting of Leukemia, Lymphoma, and Myeloma.

72. The method of claim 70, wherein the hematological cancer is selected from the group consisting of acute lymphoblastic leukemia or mantle cell lymphoma.

73. The method of any one of claim 69, wherein the cancer is a solid tumor.

74. The method of claim 73, wherein the solid tumor is selected from the group consisting of ovarian cancer, mesothelioma, brain cancer, liver cancer, kidney cancer, lung cancer, breast cancer, prostate cancer, throat cancer, thyroid cancer, colon cancer, testicular cancer, and skin cancer.

75. The method of any one of claims 69-74, wherein the cancer expresses CD19.

76. The cell of any one of claims 1-28 or polynucleotide of any one of claims 29-54, wherein the MHC class II transactivator protein is class II MHC transactivator 3


SEQUENCE LISTING (41 sequences)

SEQ ID NO: 1 - protein, 88 aa, unidentified
  source 1..88: note = Herpes Simplex Virus
MSWALEMADTFLDTMRVGPRTYADVRDEINKRGREDREAARTAVHDPERPLLRSPGLLPEIAPNASLGVAHRRTGGTVTDSPRNPVTR

SEQ ID NO: 2 - protein, 183 aa, Homo sapiens
MDLLIRLGFLLMCALPTPGERSSRDPKTLLSLSPRQQACVPRTKSHRPVCYNDTGDCTDADDSWKQLGEDFAHQCLQAAKKRPKTHKSRPNDRNLEGRLTCQRVRRLLPCDLDIHPSHRLLTLMNNCVCDGAVWNAFRLIERHGFFAVTLYLCCGITLLVVILALLCSITYESTGRGIRRCGS

SEQ ID NO: 3 - protein, 60 aa, unidentified
  source 1..60: note = Epstein-Barr virus
MVHVLERALLEQQSSACGLPGSSTETRPSHPCPEDPDVSRLRLLLVVLCVLFGLLCLLLI

SEQ ID NO: 4 - DNA, 1089 bp, synthetic construct
  source 1..1089: mol_type = other DNA
atgctggtcatggcgccccgaaccgtcctcctgctgctctcggcggccctggccctgaccgagacctgggccggctcccactccatgaggtatttctacacctccgtgtcccggcccggccgcggggagccccgcttcatctcagtgggctacgtggacgacacgcagttcgtgaggttcgacagcgacgccgcgagtccgagagaggagccgcgggcgccgtggatagagcaggaggggccggaatattgggaccggaacacacagatctgcaagaccaacacacagactgaccgagagagcctgcggaacctgcgcggctactacaaccagagcgaggccgggtctcacaccctccagaggatgtacggctgcgacgtggggccggacgggcgcctcctccgcgggcataaccagttcgcctacgacggcaaggattacatcgccctgaacgaggacctgagctcctggaccgcggcggacaccgcggctcagatcacccagcgcaagtgggaggcggcccgtgtggcggagcagctgagaacctacctggagggcacgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagacagaaccttccagaagtgggcagctgtggtggtgccttctggagaagagcagagatacacatgccatgtacagcatgaggggctgccgaagcccctcaccctgagatgggagccatcttcccagtccaccgtccccatcgtgggcattgttgctggcctggctgtcctagcagttgtggtcatcggagctgtggtcgctgctgtgatgtgtaggaggaagagctcaggtggaaaaggagggagctactctcaggctgcgtccagcgacagtgcccagggctctgatgtgtctctcacagcttga

SEQ ID NO: 5 - DNA, 360 bp, synthetic construct
  source 1..360: mol_type = other DNA
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaa

SEQ ID NO: 6 - DNA, 768 bp, synthetic construct
  source 1..768: mol_type = other DNA
atgatcctaaacaaagctctgctgctgggggccctcgctctgaccaccatgatgagcccttgtggaggtgaaggcattgtggctgaccacgttgcctcttgtggtgtaaacttgtaccagttttacggtccctctggccagtacacccatgaatttgatggagatgaggagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatgaggttcctgaggtcacagtgttttccaagtctcccgtgacactgggtcagcccaacaccctcatttgtcttgtggacaacatctttcctcctgtggtcaacatcacatggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactgggagcctgagattccagcccctatgtcagagctcacagagactgtggtctgcaccctggggttgtctgtgggcctcgtgggcattgtggtgggcactgtcttcatcatccaaggcctgcgttcagttggtgcttccagacaccaagggccattgtga

SEQ ID NO: 7 - DNA, 3396 bp, synthetic construct
  source 1..3396: mol_type = other DNA
atgcgttgcctggctccacgccctgctgggtcctacctgtcagagccccaaggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcatagagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcagcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatga

SEQ ID NO: 8 - DNA, 2800 bp, synthetic construct
  source 1..2800: mol_type = other DNA
atggcaacacaggcgtatactgagctacaggcagccccgccaccatcccagccgccacaggccccgccacaagcccagccccagccgccaccgccaccacccccagcggcaccccagcccccgcagccacccaccgctgctgccacccctcagccccaatatgtcaccgagctgcagagcccccagccccaggcacagccaccgggtggccagaagcagtacgtgacggagctcccggctgtacccgcaccctcgcagccaaccggtgcacccaccccttcgcctgcaccccagcagtacatcgtggtcactgtctctgaaggtgccatgcgggccagcgagacagtgtcggaggccagccccggctccaccgccagccagaccggcgttcctactcaggtggttcagcaggtgcagggcacccagcagcggctgctggtccagacgagcgtgcaggccaagccaggccacgtgtcgcccctccagctgaccaacatccaagtgccccagcaggctcttcccacgcagcgtctggtggtgcagagcgcagccccaggcagcaaaggtggccaggtctccctgacggtccatggtacccagcaggtgcactcgcccccagagcagtcgccggtgcaggccaacagctcttccagcaagacagccggggcccccacgggcacagtgccacagcagctgcaggtccacggcgtccagcagagtgtccccgtcacccaagagagatctgtggtccaggccactccacaagcgcccaaacccggcccggtgcagccgctgaccgtgcagggcctccagccagtccacgtggctcaagaggtgcagcagctccagcaggtgcccgtcccacacgtgtactccagccaggtgcagtatgtggagggcggcgatgccagctacacggccagtgccatccgttccagcacctactcctatcccgagacgccgctgtacacgcagacggcaagcaccagctactacgaggccgcaggcacggccacccaggtcagcacccccgccacctcccaggcggtggccagcagtggctccatgcccatgtacgtgtccggcagccaggtcgtcgccagctccgccagcactggggctggggccagcaacagcagcggaggtggtggcagtggtggtggcggcggcggcgggggaggcggtggcgggggtggcagtggcagcaccggaggcggcggcagcggagcaggcacctacgtgatccaaggcggctacatgctgggcagtgccagccagtcttactctcacaccacccgtgcctcgccagccacggtccagtggctcctggacaactatgagacggctgagggcgtgagtctgccacggagcaccctctactgccactacttactgcactgccaggagcagaagctggagcccgtcaacgccgcctccttcggcaagctcatccgctccgtcttcatgggcctgcgaacccgccgtctgggcaccaggggcaactccaagtaccactactatggcctgcgcatcaaggccagctcacccctgctgcggctgatggaggaccagcagcacatggccatgcggggccagcccttctcgcagaagcagaggctcaagcccatccagaagatggaaggcatgaccaacggcgtggcggtggggcagcagccgagcacggggctgtcggacatcagcgcccaggtgcagcagtaccagcaatttttggatgcctctcggagcctccctgacttcacagagctcgacctccagggcaaggtgctgcctgagggcgtcgggcccggggacatcaaagccttccaggtcctgtaccgggaacactgtgaggccattgtcgacgtcatggtgaacctgcagttcaccctggtggagacgctgtggaagaccttctggaggtacaacctcagccagcccagtgaggcgccaccgctggctgtacatgacgaggccgagaagcgactgcccaaagccatcctggtgctcctctccaagttcgagcccgtgctccaatggaccaagcactgtgacaacgtgctgtaccagggcctggtggaaatcctcattcccgacgtgctgcggcccatccccagtgccttgacccaagcgatccggaactttgccaagagcctggagagctggctcacccacgccatggtcaacatccccgaggagatgctgcgggtgaaggtggccgcggctggcgccttcgcgcagacactgcggcgctacacgtcgctcaaccacctggcgcaggcggcgcgcgctgtgctgcagaacaccgcacagatcaaccagatgctgagcgacctcaaccgcgtggacttcgccaacgtgcaggagcaggcctcgtgggtgtgccgctgcgaggaccgcgtggtgcagcggctggagcaggacttcaaggtgacgctgcagcagcagaactcgctggagcagtgggcggcctggctggacggcgtggtgagccaggtgctcaagccctaccagggcagcgccggcttccccaaggccgccaagctcttcctcctcaagtggtccttctacagctccatggtgatccgggacctgaccctgcgcagcgccgccagcttcggttccttccacctcatccggctgctctacgacgagtacatgtactacctgatcgagcaccgcgtagcccaggccaagggcgagacccccatcgccgtcatgggcgagttcgccaatctggccacctccctgaaccccctggaccccgacaaagacgaggaggaagaagaggaggaggagagcg

SEQ ID NO: 9 - DNA, 783 bp, synthetic construct
  source 1..783: mol_type = other DNA
atggagcttacccagcctgcagaagacctcatccagacccagcagacccctgcctcagaacttggggaccctgaagaccccggagaggaggctgcagatggctcagacactgtggtcctcagtctctttccctgcacccctgagcctgtgaatcctgaaccggatgccagtgtttcctctccacaggcaggcagctccctgaagcactccaccactctcaccaaccggcagcgagggaacgaggtgtcagctctgccggccaccctagactccctgtccatccaccagctcgcagcacagggggagctggaccagctgaaggagcatttgcggaaaggtgacaacctcgtcaacaagccagacgagcgcggcttcacccccctcatctgggcctccgcctttggagagattgagaccgttcgcttcctgctggagtggggtgccgacccccacatcctggcaaaagagcgagagagcgccctgtcgctggccagcacaggcggctacacagacattgtggggctgctgctggagcgtgacgtggacatcaacatctatgattggaatggagggacgccactgctgtacgctgtgcgcgggaaccacgtgaaatgcgttgaggccttgctggcccgaggcgctgacctcaccaccgaagccgactctggctacaccccgatggaccttgccgtggccctgggataccggaaagtgcaacaggtgatcgagaaccacatcctcaagctcttccagagcaacctggtgcccgctgaccctgagtga

SEQ ID NO: 10 - DNA, 693 bp, synthetic construct
  source 1..693: mol_type = other DNA
atgaccatggaatctggagccgagaaccagcagagtggagatgcagctgtaacagaagctgaaaaccaacaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagtcttcctgtaaggacttaaaaagacttttctccggaacacagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacttcctacacagcctgctgaagaagcagcacgaaagagagaggtccgtctaatggagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatcagattaa

SEQ ID NO: 11 - DNA, 1044 bp, synthetic construct
  source 1..1044: mol_type = other DNA
atggagcagtatacagcaaacagcaatagttcgacagagcagattgttgtccaggcaggacagattcagcagcagcagcagggtggtgtcactgctgtgcagttgcagactgaggcccaggtggcatccgcctcaggccagcaagtccagaccctccaggtagtccaagggcagccattaatggtgcaggtcagtggaggccagctaatcacatcaactggccaacccatcatggtccaggctgtccctggtggacaaggtcaaaccatcatgcaagtacctgtttctggaacacagggtttgcagcaaatacagttggtcccacctggacagatccagatccagggtggacaggctgtgcaggtgcagggccagcagggccagacccagcagatcatcatccagcagccccagacggctgtcactgctggccagactcagacacagcagcagattgctgtccagggacagcaagtggcacagactgctgaagggcagaccatcgtctatcaaccagttaatgcagatggcaccattctccagcaagttacagtccctgtttcaggcatgatcactatcccagcagccagtttggcaggagcacagattgttcaaacaggagccaataccaacacaaccagcagtgggcaagggactgtcactgtgacactaccagtggcaggcaatgtggtcaattcaggagggatggtcatgatggttcctggggctggctctgtgcctgctatccaaagaatccctctacctggagcagagatgcttgaagaagagcctctctacgtgaatgccaaacaataccaccgtattcttaagaggaggcaagcccgagctaaactagaggcagaagggaaaattccaaaggagagaaggaaatacctgcatgagtctcggcaccgtcatgccatggcacggaagcgtggtgaaggtggacgatttttctctccaaaggaaaaggatagtccccatatgcaggatccaaaccaagccgatgaagaagcaatgacacagatcatccgagtgtcctaa

SEQ ID NO: 12 - DNA, 1065 bp, synthetic construct
  source 1..1065: mol_type = other DNA
atgtccacagaaggaggatttggtggtactagcagcagtgatgcccagcaaagcctacagtcgttctggcctcgggtcatggaagaaatccggaatttaacagtgaaagacttccgagtgcaggaactcccactggctcgtattaagaagattatgaaactggatgaagatgtgaagatgatcagtgcagaagcgcctgtactctttgccaaggcagcccagatttttatcacagagttgactcttcgagcctggattcacacagaagataacaagcgccggactctacagagaaatgatatcgccatggcaattacaaaatttgatcagtttgattttctcatcgatattgttccaagagatgaactgaaacctccaaagcgtcaggaggaggtgcgccagtctgtaactcctgccgagccagtccagtactatttcacgctggctcagcaacccaccgctgtccaagtccagggccagcagcaaggccagcagaccaccagctccacgaccaccatccagcctgggcagatcatcatcgcacagcctcagcagggccagaccacacctgtgacaatgcaggttggagaaggtcagcaggtgcagattgtccaggctcagccacagggtcaagcccaacaggcccagagtggcactggacagaccatgcaggtgatgcagcagatcatcactaacacaggagagatccagcagatcccggtgcagctgaatgccggccagctgcagtatatccgcttagcccagcctgtatcaggcactcaagttgtgcagggacagatccagacacttgccaccaatgctcaacaggggcaaagaaatgcaagtcaggggaagcctcgaaggtgcctgaaagaaaccttacagattacacagacagaggtccagcaaggacagcagcagttcagccagttcacagatggacagcagctctaccagatccagcaagtcaccatgcctgcgggccaggacctcgcccagcccatgttcatccagtcagccaaccagccctccgacgggcaggccccccaggtgaccggcgactga

SEQ ID NO: 13 - DNA, 58 bp, synthetic construct
  source 1..58: mol_type = other DNA
gcttctgctgatgagatttattttcaagagaaataaatctcatcagcagaagtttttt

SEQ ID NO: 14 - DNA, 2757 bp, synthetic construct
  source 1..2757: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagctctagagccatggtacacgtattggaacgggcgcttttggagcagcaaagctccgcgtgcggactcccaggttcatccacggaaacacgcccatctcatccctgcccggaggaccccgatgtatcacgacttaggctcttgctggtcgtactttgcgtgctctttggacttctgtgcctcctgctcatcggctcaggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 15 - DNA, 1977 bp, synthetic construct
  source 1..1977: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagctctagagccatggtacacgtattggaacgggcgcttttggagcagcaaagctccgcgtgcggactcccaggttcatccacggaaacacgcccatctcatccctgcccggaggaccccgatgtatcacgacttaggctcttgctggtcgtactttgcgtgctctttggacttctgtgcctcctgctcatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 16 - DNA, 311 bp, synthetic construct
  source 1..311: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacaacaggattcacggatcagcttcaagagagctgatccgtgaatcctgttgtttttttt

SEQ ID NO: 17 - DNA, 4107 bp, synthetic construct
  source 1..4107: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagctctagagccatggccctccctgtcaccgccctgctgcttccgctggctcttctgctccacgccgctcggcccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggtggaggtggcagcggaggaggtgggtccggcggtggaggaagcggcggtggaggaagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaataccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtccggaggcggcggagagggcagaggaagtcttctaacatgcggtgacgtggaggagaatcccggccctaggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 18 - DNA, 3315 bp, synthetic construct
  source 1..3315: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagctctagagccatggccctccctgtcaccgccctgctgcttccgctggctcttctgctccacgccgctcggcccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggtggaggtggcagcggaggaggtgggtccggcggtggaggaagcggcggtggaggaagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaataccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 19 - DNA, 311 bp, synthetic construct; identical to SEQ ID NO: 16
  source 1..311: mol_type = other DNA

SEQ ID NO: 20 - DNA, 4353 bp, synthetic construct
  source 1..4353: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagctctagagccatggccctccctgtcaccgccctgctgcttccgctggctcttctgctccacgccgctcggcccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggtggaggtggcagcggaggaggtgggtccggcggtggaggaagcggcggtggaggaagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaataccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggctcaggagagggcagaggaagtcttctaacatgcggtgacgtggaggagaatcccggccctatggtacacgtattggaacgggcgcttttggagcagcaaagctccgcgtgcggactcccaggttcatccacggaaacacgcccatctcatccctgcccggaggaccccgatgtatcacgacttaggctcttgctggtcgtactttgcgtgctctttggacttctgtgcctcctgctcatcggctcaggaggttcaggtgcaacgaacttctcattgttgaagcaagccggtgatgttgaggaaaatccgggtcctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 21 - DNA, 311 bp, synthetic construct; identical to SEQ ID NO: 16
  source 1..311: mol_type = other DNA

SEQ ID NO: 22 - DNA, 4348 bp, synthetic construct
  source 1..4348: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagctctagagccatggccctccctgtcaccgccctgctgcttccgctggctcttctgctccacgccgctcggcccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggtggaggtggcagcggaggaggtgggtccggcggtggaggaagcggcggtggaggaagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaataccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggctcaggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggttcaggtgcaacgaacttctcattgttgaagcaagccggtgatgttgaggaaaatccgggtcctatggtacacgtattggaacgggcgcttttggagcagcaaagctccgcgtgcggactcccaggttcatccacggaaacacgcccatctcatccctgcccggaggaccccgatgtatcacgacttaggctcttgctggtcgtactttgcgtgctctttggacttctgtgcctcctgctcatcggctcaggataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 23 - DNA, 311 bp, synthetic construct; identical to SEQ ID NO: 16
  source 1..311: mol_type = other DNA

SEQ ID NO: 24 - DNA, 3549 bp, synthetic construct
  source 1..3549: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagctctagagccatggccctccctgtcaccgccctgctgcttccgctggctcttctgctccacgccgctcggcccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggtggaggtggcagcggaggaggtgggtccggcggtggaggaagcggcggtggaggaagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaataccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggctcaggagagggcagaggaagtcttctaacatgcggtgacgtggaggagaatcccggccctatggtacacgtattggaacgggcgcttttggagcagcaaagctccgcgtgcggactcccaggttcatccacggaaacacgcccatctcatccctgcccggaggaccccgatgtatcacgacttaggctcttgctggtcgtactttgcgtgctctttggacttctgtgcctcctgctcatctaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 25 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 26 - protein, 42 aa, synthetic construct
KRGRKKLLYIFKQPFMRPVQTTQEEDGCSCRFPEEEEGGCEL

SEQ ID NO: 27 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 21 aa, synthetic construct
MALPVTALLLPLALLLHAARP

SEQ ID NO: 29 - protein, 21 aa, synthetic construct
METDTLLLWVLLLWVPGSTGD

SEQ ID NO: 30 - protein, 5 aa, synthetic construct
GGGGS

SEQ ID NO: 31 - protein, 12 aa, synthetic construct
GGGSGGGSGGGS

SEQ ID NO: 32 - protein, 15 aa, synthetic construct
GGGGSGGGGSGGGGS

SEQ ID NO: 33 - protein, 20 aa, synthetic construct
GGGGSGGGGSGGGGSGGGGS

SEQ ID NO: 34 - protein, 18 aa, synthetic construct
GSTSGSGKPGSGEGSTKG

SEQ ID NO: 35 - protein, 18 aa, synthetic construct
GGSSRSSSSGGGGSGGGG

SEQ ID NO: 36 - protein, 24 aa, synthetic construct
SGGGGEGRGSLLTCGDVEENPGPR

SEQ ID NO: 37 - protein, 22 aa, synthetic construct
GSGATNFSLLKQAGDVEENPGP

SEQ ID NO: 38 - protein, 6 aa, synthetic construct
HHHHHH

SEQ ID NO: 39 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 40 - protein, 123 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSN

SEQ ID NO: 41 - protein, 507 aa, synthetic construct
MALPVTALLLPLALLLHAARPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSNTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR